(19) United States
(12) Patent Application Publication
(10) Pub. No.: US 2021/0402016 A1
(43) Pub. Date: Dec. 30, 2021

(54) RADIOLABELED BOMBESIN-DERIVED COMPOUNDS FOR IN VIVO IMAGING OF GASTRIN-RELEASING PEPTIDE RECEPTOR (GRPR) AND TREATMENT OF GRPR-RELATED DISORDERS

(71) Applicant: Provincial Health Services Authority, Vancouver (CA)

(72) Inventors: Francois Benard, Vancouver, BC (CA); Kuo-Shyan Lin, Richmond, BC (CA); Etienne Rousseau, Sherbrooke (CA); Zhengxing Zhang, Vancouver, BC (CA); Joseph Lau, Richmond, BC (CA); lvica Bratanovic, Coquitlam (CA); Jutta Zeisler, Vancouver, BC (CA)

(73) Assignee: Provincial Health Services Authority, Vancouver, BC (CA)

(21) Appl. No.: 17/293,455

(22) PCT Filed: Nov. 13, 2019
(86) PCT No.: PCT/CA2019/051620

§ 371 Date: May 12, 2021

Related U.S. Application Data
(60) Provisional application No. 62/760,857, filed on Nov. 13, 2018.

(51) Int. Cl. A61K 51/08; C07K 7/08

(52) U.S. Cl. CPC A61K 51/088; C07K 7/086

(57) ABSTRACT

There is provided bombesin-derived compounds of Formula Ia (R^{X}-L-Xaa¹-Gln-Trp-Ala-Val-Xaa²-His-Xaa³-ψ-Xaa⁴-NH₂). R^{X} comprises a radionuclide chelator or a trifluoroborate-containing prosthetic group. L is a linker. Xaa¹ is D-Phe, Cpa (4-chlorophenylalanine), D-Cpa, Tpi (2,3,4,9-tetrahydro-1H-pyrido[3,4b]indol-3-carboxylic acid), D-Tpi, Nal (naphthylalanine), or D-Nal. Xaa² is Gly, N-methyl-Gly or D-Ala. Xaa³ is Leu, Pro, D-Pro, or Phe. Xaa⁴ is Pro, Phe, Tac (thiazolidine-4-carboxylic acid), Nle (norleucine), 4-oxa-L-Pro (oxazolidine-4-carboxylic acid). The symbol ψ represents a reduced peptide bond between Xaa³ and Xaa⁴ in which ψ is CH₂—N when Xaa⁴ is Pro, Tac or 4-oxa-L-Pro, or ψ is CH₂N(R) when Xaa⁴ is Phe or Nle wherein R is H or C₁-C₅ linear or branched alkyl. There is also provided the use of such compounds as imaging agents or therapeutic agents.

### FIELD OF INVENTION

The present invention relates to radiolabelled compounds for in vivo imaging or treatment of diseases or conditions characterized by expression of the gastrin releasing peptide receptor.

### BACKGROUND OF THE INVENTION

Gastrin-releasing peptide receptor (GRPR) is a G protein-coupled receptor of the bombesin (BBN) receptor family (1-3). Together with its endogenous ligand, gastrin-releasing peptide (GRP), GRPR is involved in synaptic plasticity, emotional and feeding behavior, hormone secretion, smooth muscle contraction, and cell proliferation (1-3). In normal conditions, the expression of GRPR is restricted to the central nervous system, pancreas, adrenal cortex and gastrointestinal tract (4). GRPR is also implicated in neoplastic progression, with overexpression of GRPR having been reported in many cancer subtypes including lung, head and neck, colon, kidney, ovarian, breast and prostate cancers (5). This ectopic expression in cancers makes it an attractive target for personalized therapies.

BBN is a 14 amino acid GRPR binding peptide (7-14). BBN derivatives have been radiolabeled for imaging with single photon emission computed tomography (SPECT), positron emission tomography (PET), and have also been radiolabeled for therapy with beta and alpha emitters (6-8). Often, a radiolabelled group is appended directly onto the structure or via a linker at the N-terminus, while modifications at the C-terminus dictate agonist/antagonist properties. For targeting GRPR, antagonists are preferred since agonists have been shown to induce gastrointestinal adverse events (10). Examples of GRPR antagonists evaluated in the clinic include: ⁶⁸Ga-RM2, ⁶⁸Ga-SB3, ⁶⁸Ga-NeoBOMB1, ⁶⁸Ga-RM26, ¹⁸F-BAY-864367, and ⁶⁴Cu-CB-TE2A-AR06 (9, 11-16).

It has been reported that reduction of the peptide bond between residues 13 and 14 of BBN to —CH₂—NH— produced a potent antagonist (17). Based on this work, others reported a series of pseudononapeptide BBN antagonists with a reduced bond (CH₂—NH or CH₂—N) between amino acid residues 13 and 14 of BBN (Leu¹³ψTac¹⁴) (18). Several of those exhibited picomolar binding affinity for murine GRPR, and some were able to inhibit growth of prostate, breast, colon, lung, liver, pancreas, ovary, kidney, and glioma cancers in preclinical models (5, 19-23).

There remains an unmet need in the field for improved tracers for the non-invasive in-vivo imaging of the GRPR. Such tracers are useful for the diagnosis of disorders related to aberrant/ectopic expression of GRPR, including but not limited to cancer (e.g. prostate cancer). There also remains an unmet need for improved radiotherapeutic agents for treatment of diseases/disorders related to aberrant/ectopic expression of GRPR, including but not limited to cancer (e.g. prostate cancer).

No admission is necessarily intended, nor should it be construed, that any of the preceding information constitutes prior art against the present invention.

### SUMMARY

Various embodiments of this disclosure relate to a compound of Formula Ia (R^{X}-L-Xaa¹-Gln-Trp-Ala-Val-Xaa²-His-Xaa³-L-Xaa⁴-NH₂) wherein: R^{X} comprises a radionuclide chelator or a trifluoroborate-containing prosthetic group; L is a linker; Xaa¹ is D-Phe, Cpa (4-chlorophenylalanine), D-Cpa, Tpi (2,3,4,9-tetrahydro-1H-pyrido[3,4b]indol-3-carboxylic acid), D-Tpi, Nal (naphthylalanine), or D-Nal; Xaa² is Gly, N-methyl-Gly or D-Ala; Xaa³ is Leu, Pro, D-Pro, or Phe; Xaa⁴ is Pro, Phe, Tac (thiazolidine-4-carboxylic acid), Nle (norleucine), 4-oxa-L-Pro (oxazolidine-4-carboxylic acid); and ψ represents a reduced peptide bond between Xaa³ and Xaa⁴ in which ψ is

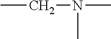

when Xaa⁴ is Pro, Tac or 4-oxa-L-Pro, or ψ is —CH₂N(R)— when Xaa⁴ is Phe or Nle wherein R is H or C₁-C₅ linear or branched alkyl.

In some embodiments, R^{X} comprises the radionuclide chelator. The radionuclide chelator may be selected from the group consisting of: DOTA and derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate; hexamethylpropyleneamineoxime; and hexakis(methoxy isobutyl isonitrile). In certain embodiments, the radionuclide chelator is selected from DOTA and DOTA derivatives.

In some embodiments, R^{X} further comprises a radiometal, a radionuclide-bound metal, or a radionuclide-bound metal-containing prosthetic group, and wherein the radiometal, the radionuclide-bound metal, or the radionuclide-bound metal-containing prosthetic group is chelated to the radionuclide-chelator complex. The radiometal, the radionuclide-bound metal, or the radionuclide-bound metal-containing prosthetic group may be: ⁶⁸Ga, ⁶¹Cu, ⁶⁴Cu, ⁶⁷Cu, ⁶⁷Ga, ¹¹¹In, ⁴⁴Sc, ⁸⁶Y, ⁸⁹Zr, ⁹⁰Nb ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, or [¹⁸F]AlF. In certain embodiments, the radiometal, the radionuclide-bound metal, or the radionuclide-bound metal-containing prosthetic group is: ⁶⁸Ga, ⁶¹Cu, ⁶⁴Cu, ⁶⁷Cu, ⁶⁷Ga, ¹¹¹In, ⁴⁴Sc, ⁸⁶Y, ¹⁷⁷Lu, ⁹⁰Y, ¹⁴⁹Tb, ¹⁵²Tb, ¹⁵⁵Tb, ¹⁶¹Tb, ²²⁵Ac, ²¹³Bi, or ²¹²Bi.

In some embodiments, R^{X} comprises one or more than one trifluoroborate-containing prosthetic group. R^{X} may comprise one or more than one R¹R²BF₃ group, wherein: each R¹ is independently

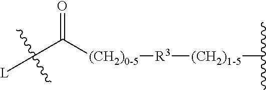

wherein each R³ is independently absent,

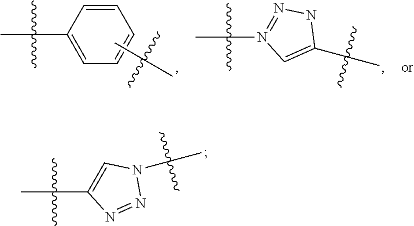

and each R²BF₃ is independently:

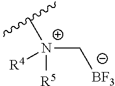

wherein each R⁴ is independently a C₁-C₅ linear or branched alkyl group and each R⁵ is independently a C₁-C₅ linear or branched alkyl group,

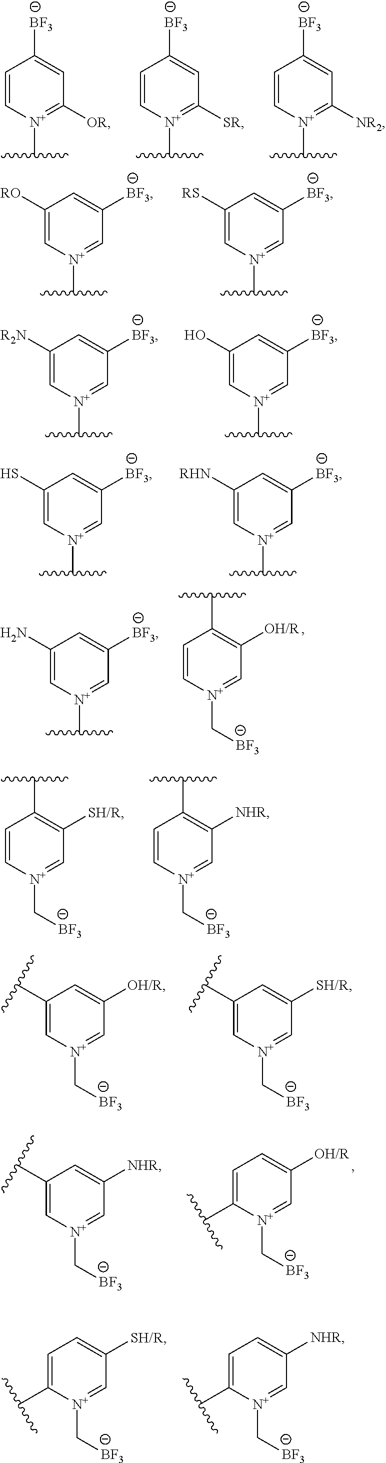

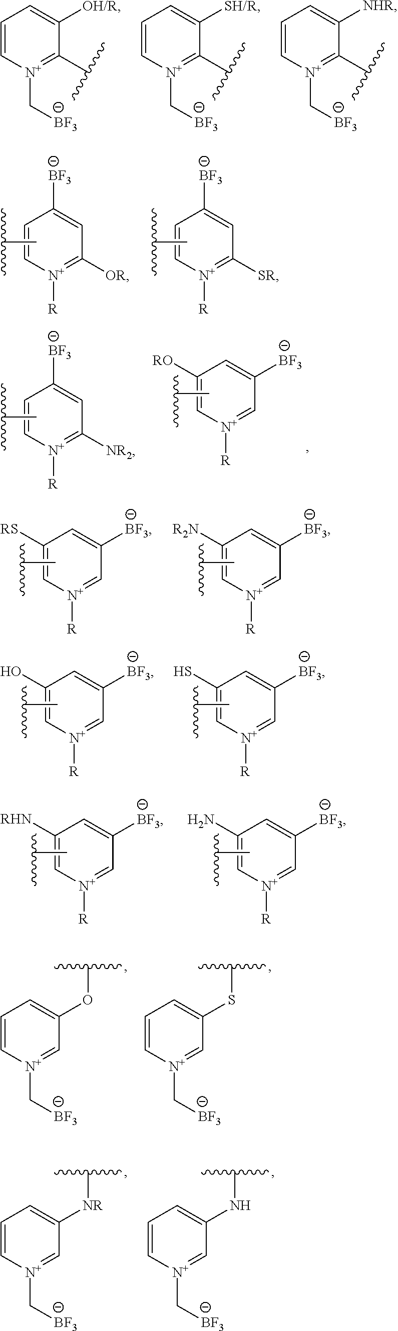

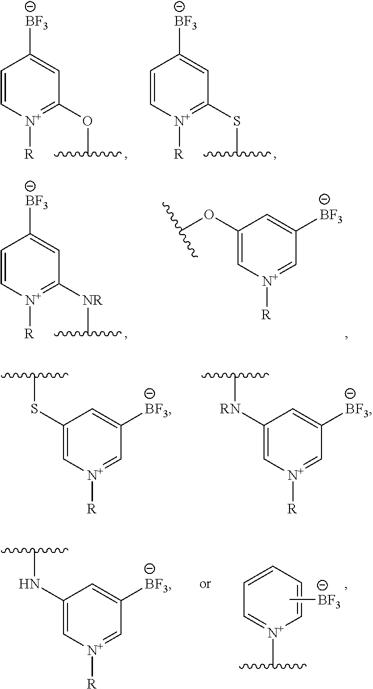

in which the R in each pyridine substituted —OR, —SR, —NR—, —NHR or —NR₂ is independently a branched or linear C₁-C₅ alkyl. R^{X} may comprise one or more than one R¹R²BF₃, wherein: each R¹ is independently

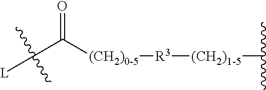

wherein each R³ is independently absent,

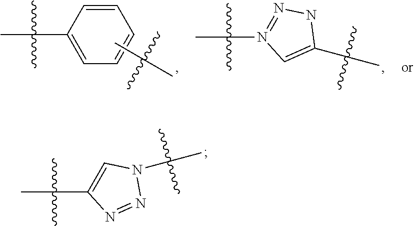

and each R²BF₃ is independently:

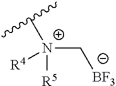

wherein each R⁴ is independently a C₁-C₅ linear or branched alkyl group and each R⁵ is independently a C₁-C₅ linear or branched alkyl group,

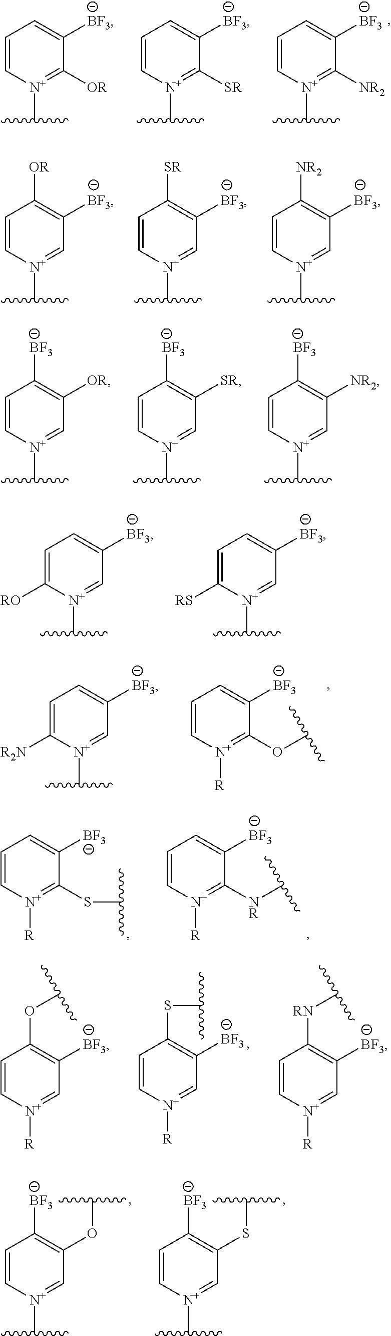

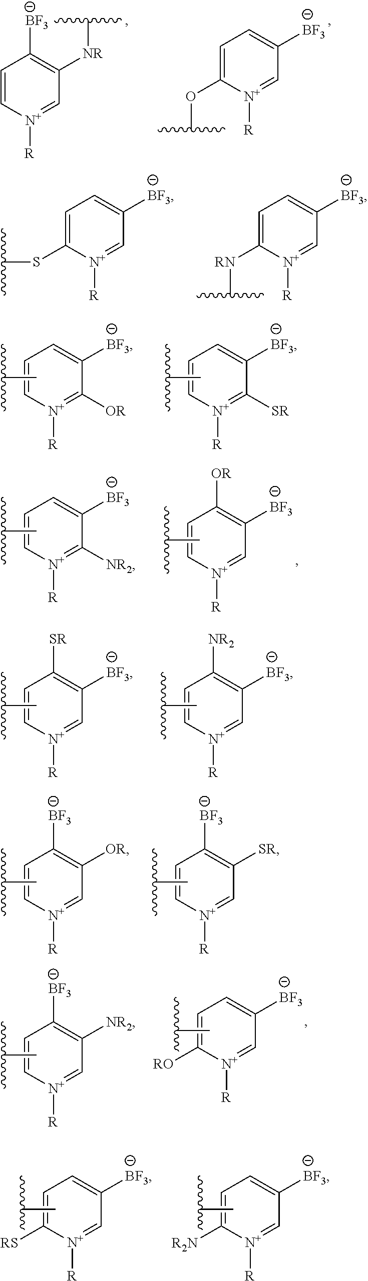

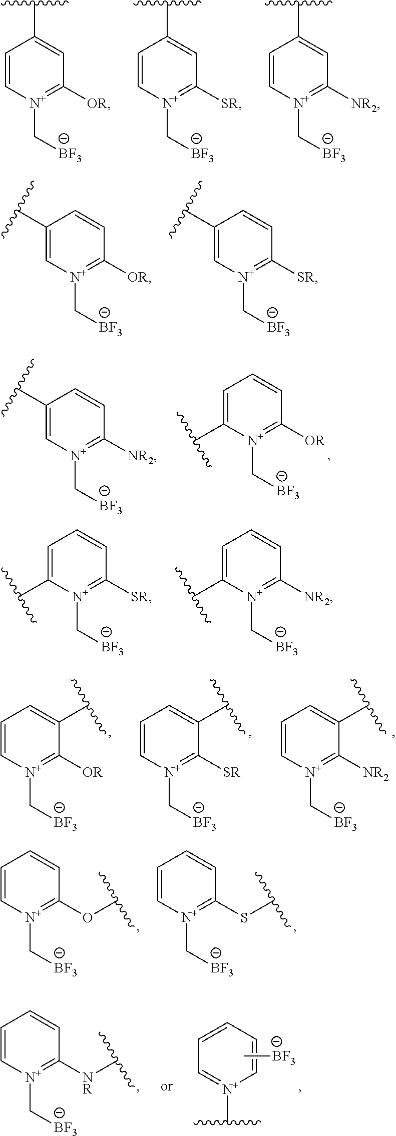

in which the R in each pyridine substituted —OR, —SR, —NR—, —NHR or —NR_{z} is independently a branched or linear C₁-C₅ alkyl. In certain embodiments, the R^{X} comprises a single R¹R²BF₃ group. In certain embodiments, the R^{X} comprises two R¹R²BF₃ groups. The trifluoroborate-containing prosthetic group(s) may comprise ¹⁸F.

In some embodiments, the linker is a peptide linker (Xaa⁵)₁₋₄, wherein each Xaa⁵ is independently a proteinogenic or non-proteinogenic amino acid residue. In some embodiments, the linker is a peptide linker (Xaa⁵)₁₋₄, wherein each Xaa⁵ is independently a proteinogenic amino acid residue or is a non-proteinogenic amino acid residue, wherein each peptide backbone amino group is independently optionally methylated, and wherein each non-proteinogenic amino acid residue is independently selected from the group consisting of a D-amino acid of a proteinogenic amino acid, N^{ε}, N^{ε}, N^{ε}-trimethyl-lysine, 2,3-diaminopropionic acid (Dap), 2,4-diaminobutyric acid (Dab), ornithine (Orn), homoarginine (hArg), 2-amino-4-guanidinobutyric acid (Agb), 2-amino-3-guanidinopropionic acid (Agp), 4-(2-aminoethyl)-1-carboxymethyl-piperazine (Acp), β-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-aminoadipic acid (2-Aad), 3-aminoadipic acid (3-Aad), cysteic acid, tranexamic acid, p-aminomethylaniline-diglycolic acid (pABzA-DIG), 4-amino-1-carboxymethyl-piperidine (Pip), NH₂(CH₂)₂O(CH₂)₂C(O)OH, NH₂(CH₂)₂[O(CH₂)₂]₂C(O)OH (dPEG2), NH₂(CH₂)₂[O(CH₂)₂]₃C(O)OH, NH₂(CH₂)₂[O(CH₂)₂]₄C(O)OH, NH₂(CH₂)₂[O(CH₂)₂]₅C(O)OH, and NH₂(CH₂)₂[O(CH₂)₂]₆C(O)OH. In some embodiments, the linker is p-aminomethylaniline-diglycolic acid (pABzA-DIG), 4-amino-(1-carboxymethyl)piperidine (Pip), 9-amino-4,7-dioxanonanoic acid (dPEG2) or 4-(2-aminoethyl)-1-carboxymethyl-piperazine (Acp). In certain embodiments, the linker is pABzA-DIG or Pip.

In some embodiments, Xaa¹ is D-Phe. In some embodiments, Xaa² is Gly. In some embodiments, Xaa³ is Leu. In some embodiments, Xaa⁴ is Pro, Tac or 4-oxa-L-Pro. In some embodiments, Xaa⁴ is Pro. In some embodiments, Xaa¹ is D-Phe, Xaa² is Gly, Xaa³ is Leu, and Xaa⁴ is Pro.

Various embodiments of the disclosure relate to a compound, the compound having the following chemical structure or a salt or solvate thereof, optionally chelated with radionuclide X:

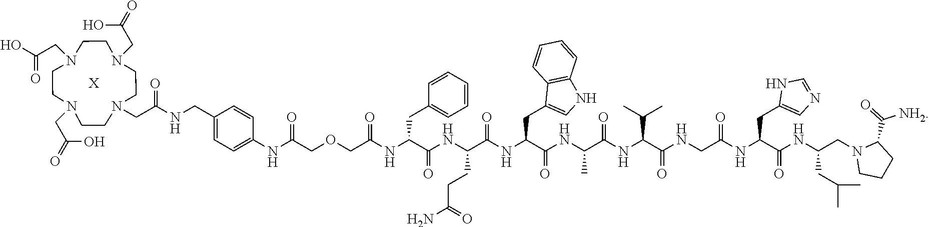

In some embodiments, X is: ⁶⁸Ga, ⁶⁴Cu, ⁶⁷Cu, ⁶⁷Ga, ¹¹¹In, ¹⁷⁷Lu, ⁹⁰Y, or ²²⁵Ac. In other embodiments, X is X is: ⁶⁸Ga or ¹⁷⁷Lu.

Various embodiments of the disclosure relate to a compound, the compound having the following chemical structure or a salt or solvate thereof, optionally chelated with radionuclide X:

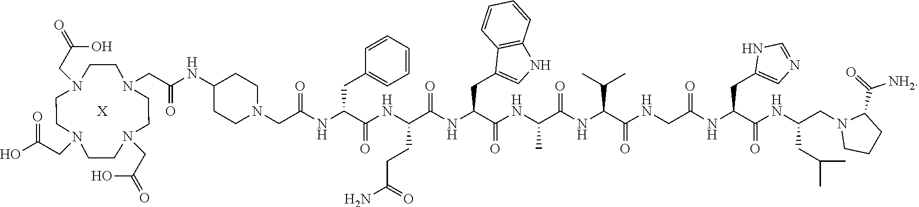

In some embodiments, X is: ⁶⁸Ga, ⁶⁴Cu, ⁶⁷Cu, ⁶⁷Ga, ¹¹¹In, ¹⁷⁷Lu, ⁹⁰Y, or ²²⁵Ac. In other embodiments, X is X is: ⁶⁸Ga or ¹⁷⁷Lu.

### BRIEF DESCRIPTION OF THE DRAWINGS

The features of the invention will become apparent from the following description in which reference is made to the appended drawings wherein:

FIG. 1 shows the chemical structures of prior art compounds RC-3950-II (top) and Ga-NeoBOMB1 (bottom).

FIG. 2 shows a graph of intracellular calcium efflux in PC-3 cells. Cells were incubated with 50 nM of Ga-ProBOMB1, H-3042 ([D-Phe⁶, Leu-NHEt¹³, des-Met¹⁴]Bombesin(6-14)), Bombesin, ATP, or buffer control. ***p≤0.001 compared with buffer control.

FIG. 3 shows maximum intensity projections for PET/CT and PET alone with (A)⁶⁸Ga-NeoBOMB1 and (B)⁶⁸Ga-ProBOMB1 acquired at 1 or 2 h p.i. in mice bearing PC-3 tumor xenografts. Blocking was performed with co-injection of 100 μg of [D-Phe⁶, Leu-NHEt¹³, des-Met¹⁴]Bombesin(6-14). The scale bar is in units of % ID/g (percent injected dose per gram of tissues) from 0 to 15 with the white color at the bottom of the bar representing 0% ID/g and the black color at the top of the bar representing 15% ID/g): t=tumor; l=liver; p=pancreas; b=bowel; bl=bladder.

FIG. 4 is a graph showing biodistribution of ⁶⁸Ga-NeoBOMB1 and ⁶⁸Ga-ProBOMB1 in selected tissues at multiple time points (*p≤0.05; **p≤0.01; ***p≤0.001).

FIG. 5 is a graph showing biodistribution of ⁶⁸Ga-ProBOMB1 at 60 minute p.i. with or without co-injection of 100 μg of [D-Phe⁶, Leu-NHEt¹³, des-Met¹⁴]Bombesin(6-14) (***p≤0.001).

FIG. 6 shows HPLC chromatograms indicating plasma stability of ⁶⁸Ga-ProBOMB1 at 5 min p.i. Minor metabolite peak M1 was observed at t_{R}=2.72 min on HPLC chromatograms.

FIG. 7 shows a graph of absorbed doses per unit of injected activity in mice for ⁶⁸Ga-NeoBOMB1 and ⁶⁸Ga-ProBOMB1.

FIG. 8 shows representative displacement curves of [¹²⁵I-Tyr⁴]Bombesin by [D-Phe⁶, Leu-NHEt¹³, des-Met¹⁴]Bombesin(6-14) (H3042), Ga-NeoBOMB1, and Ga-ProBOMB1.

FIG. 9 is a graph representing FLIPR Calcium 6 release assay in PC-3 cells. Cells were incubated with Ga-ProBOMB1, H-3042 ([D-Phe⁶, Leu-NHEt¹³, des-Met¹⁴]Bombesin(6-14)], Bombesin, ATP, or PBS control. The y-axis is relative fluorescence unit (RFU) and the x-axis is time (sec).

FIG. 10 is a composite of graphs representing uptake of the ⁶⁸Ga-NeoBOMB1 as a function of time for pancreas, blood, kidneys and PC-3 tumors. The total number of decays per unit injected dose is calculated by multiplying the area under the curve by the phantom organ mass. The y-axis is percentage injected dose per gram of tissue (% ID/g) and the x-axis is time (h).

FIG. 11 is a composite of graphs representing uptake of the ⁶⁸Ga-ProBOMB1 as a function of time for pancreas, blood, kidneys and PC-3 tumors. The total number of decays per unit injected dose is calculated by multiplying the area under the curve by the phantom organ mass. The y-axis is percentage injected dose per gram of tissue (% ID/g) and the x-axis is time (h).

FIG. 12 shows maximum intensity projections for PET/CT and PET alone with ⁶⁸Ga-ProBOMB2 acquired at 1 h, 2 h, and 1 h block p.i. in mice bearing PC-3 tumor xenografts. Blocking was performed with co-injection of 100 μg of [D-Phe⁶, Leu-NHEt¹³, des-Met¹⁴]Bombesin(6-14). The scale bar is in units of % ID/g (percent injected dose per gram of tissues) from 0 to 5 with the white color at the bottom of the bar representing 0% ID/g and the black color at the top of the bar representing 5% ID/g.

FIG. 13 is a graph showing biodistribution of ⁶⁸Ga-ProBOMB2 at 60 minutes and 120 minutes p.i. in mice bearing PC-3 prostate cancer xenografts.

FIG. 14 shows HPLC chromatograms indicating in-vivo plasma stability of ⁶⁸Ga-ProBOMB2 in mice at 5 min and 15 min p.i. Minor metabolite peak M1 was observed at t_{R}=2.7 min on HPLC chromatograms.

FIG. 15 is a composite graph showing representative displacement curves of [¹²⁵I-Tyr⁴]Bombesin by Ga-ProBOMB2 (left) and Lu-ProBOMB2 (right) for human GRPR on PC-3 cells.

FIG. 16 is a composite graph showing representative displacement curves of [¹²⁵I-Tyr⁴]Bombesin by Ga-ProBOMB2 (left) and Lu-ProBOMB2 (right) for murine GRPR on Swiss 3T3 cells.

FIG. 17 is a time-activity curve of ⁶⁸Ga-ProBOMB2 for blood, kidneys, muscle, bone, and PC-3 tumor. These curves are obtained from dynamic PET imaging scan of ⁶⁸Ga-ProBOMB2 in PC-3 tumor-bearing mice.

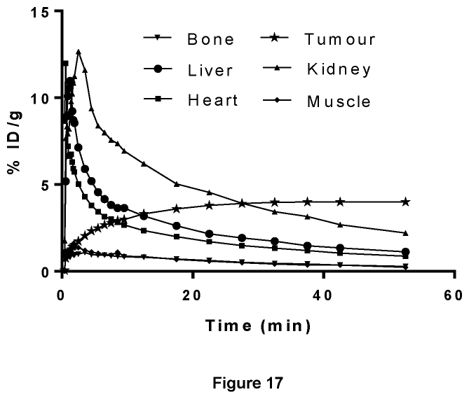

### DETAILED DESCRIPTION

As used herein, the terms “comprising,” “having”, “including” and “containing,” and grammatical variations thereof, are inclusive or open-ended and do not exclude additional, unrecited elements and/or method steps, even if a feature/component defined as a part thereof consists or consists essentially of specified feature(s)/component(s). The term “consisting essentially of” if used herein in connection with a compound, composition, use or method, denotes that additional elements and/or method steps may be present, but that these additions do not materially affect the manner in which the recited compound, composition, method or use functions. The term “consisting of” if used herein in connection with a feature of a compound, composition, use or method, excludes the presence of additional elements and/or method steps in that feature. A compound, composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the context clearly dictates otherwise. The use of the word “a” or “an” when used herein in conjunction with the term “comprising” may mean “one,” but it is also consistent with the meaning of “one or more,” “at least one” and “one or more than one.”

In this disclosure, the recitation of numerical ranges by endpoints includes all numbers subsumed within that range including all whole numbers, all integers and, where suitable, all fractional intermediates (e.g., 1 to 5 may include 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, et cetera.

As used herein, the terms “treat”, “treatment”, “therapeutic” and the like includes ameliorating symptoms, reducing disease progression, improving prognosis and reducing recurrence.

As used herein, the term “diagnostic agent” includes an “imaging agent”. As such, a “diagnostic radionuclide” includes radionuclides that are suitable for use in imaging agents.

The term “subject” refers to an animal (e.g. a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like). In some embodiments, the subject is a human.

The compounds disclosed herein may also include base-free forms, solvates, salts or pharmaceutically acceptable salts thereof. Unless otherwise specified or indicated, the compounds claimed and described herein are meant to include all racemic mixtures and all individual enantiomers or combinations thereof, whether or not they are explicitly represented herein.

The compounds disclosed herein may be shown as having one or more charged groups, may be shown with ionizable groups in an uncharged (e.g. protonated) state or may be shown without specifying formal charges. As will be appreciated by the person of skill in the art, the ionization state of certain groups within a compound (e.g. without limitation, CO₂H, and the like) is dependent, interalia, on the pKa of that group and the pH at that location. For example, but without limitation, a carboxylic acid group (i.e. COOH) would be understood to usually be deprotonated (and negatively charged) at neutral pH and at most physiological pH values, unless the protonated state is stabilized.

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g. without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. As used herein, “pharmaceutically acceptable” means suitable for in vivo use in a subject, and is not necessarily restricted to therapeutic use, but also includes diagnostic use. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety.

As used herein, in the context of an alkyl group of a compound, the term “linear” may be used as it is normally understood to a person of skill in the art and generally refers to a chemical entity that comprises a skeleton or main chain that does not split off into more than one contiguous chain. Non-limiting examples of linear alkyls include methyl, ethyl, n-propyl, and n-butyl.

As used herein, the term “branched” may be used as it is normally understood to a person of skill in the art and generally refers to a chemical entity that comprises a skeleton or main chain that splits off into more than one contiguous chain. The portions of the skeleton or main chain that split off in more than one direction may be linear. Non-limiting examples of a branched alkyl group include tert-butyl and isopropyl.

As used herein, the term “saturated” when referring to a chemical entity may be used as it is normally understood to a person of skill in the art and generally refers to a chemical entity that comprises only single bonds, and may include linear and/or branched groups. Non-limiting examples of a saturated linear or branched C₁-C₅ alkyl group includes methyl, ethyl, n-propyl, i-propyl, sec-propyl, n-butyl, i-butyl, sec-butyl, t-butyl, n-pentyl, i-pentyl, sec-pentyl, t-pentyl, 1,2-dimethylpropyl, and 2-ethylpropyl.

The wavy line “ [symbol] ” symbol shown through or at the end of a bond in a chemical formula (e.g. in the definition R²BF₃ of Formula 1a) is intended to define the R group on one side of the wavy line, without modifying the definition of the structure on the opposite side of the wavy line. Where an R group is bonded on two or more sides (e.g. R¹ and R³ of Formula 1a), any atoms shown outside the wavy lines are intended to clarify orientation of the R group. As such, only the atoms between the two wavy lines constitute the definition of the R group. When atoms are not shown outside the wavy lines, or for a chemical group shown without wavy lines but does have bonds on multiple sides (e.g. —C(O)NH—, and the like.), the chemical group should be read from left to right matching the orientation in the formula that the group relates to (e.g. for formula —R^{d}-R^{e}-R^{f}—, the definition of R^{e} as —C(O)NH— would be incorporated into the formula as —R^{d}—C(O)NH—R^{f}— not as —R^{d}—NHC(O)—R^{f}—) unless another orientation is clearly intended.

In the structures provided herein, hydrogen may or may not be shown. In some embodiments, hydrogens (whether shown or implicit) may be protium (i.e. ¹H), deuterium (i.e. ²H) or combinations of ¹H and ²H. Methods for exchanging ¹H with ²H are well known in the art. For solvent-exchangeable hydrogens, the exchange of ¹H with ²H occurs readily in the presence of a suitable deuterium source, without any catalyst. The use of acid, base or metal catalysts, coupled with conditions of increased temperature and pressure, can facilitate the exchange of non-exchangeable hydrogen atoms, generally resulting in the exchange of all ¹H to ²H in a molecule.

The term “Xaa” refers to an amino acid residue in a peptide chain or an amino acid that is otherwise part of a compound. Amino acids have both an amino group and a carboxylic acid group, either or both of which can be used for covalent attachment. In attaching to the remainder of the compound, the amino group and/or the carboxylic acid group may be converted to an amide or other structure; e.g. a carboxylic acid group of a first amino acid is converted to an amide (i.e. a peptide bond) when bonded to the amino group of a second amino acid. As such, Xaa may have the formula —N(R^{a})R^{b}C(O)—, where R^{a} and R^{b} are R-groups. R^{a} will typically be hydrogen or methyl or R^{a} and R^{b} may form a cyclic structure. The amino acid residues of a peptide may comprise typical peptide (amide) bonds and may further comprise bonds between side chain functional groups and the side chain or main chain functional group of another amino acid. For example, the side chain carboxylate of one amino acid residue in the peptide (e.g. Asp, Glu, etc.) may be bonded to and the amine of another amino acid residue in the peptide (e.g. Dap, Dab, Orn, Lys). Further details are provided below. Unless otherwise indicated, “Xaa” may be any amino acid, including a proteinogenic or nonproteinogenic amino acid. Non-limiting examples of nonproteinogenic amino acids are shown in Table 1 and include: D-amino acids (including without limitation any D-form of the following amino acids), ornithine (Orn), 3-(1-naphtyl)alanine (Nal), 3-(2-naphtyl)alanine (2-Nal), a-aminobutyric acid, norvaline, norleucine (Nle), homonorleucine, beta-(1,2,3-triazol-4-yl)-L-alanine, 1,2,4-triazole-3-alanine, Phe(4-F), Phe(4-Cl), Phe(4-Br), Phe(4-I), Phe(4-NH₂), Phe(4-NO₂), homoarginine (hArg), 2-amino-4-guanidinobutyricacid (Agb), 2-amino-3-guanidinopropionic acid (Agp), B-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-amino-3-(anthracen-2-yl)propanoic acid, 2-amino-3-(anthracen-9-yl)propanoic acid, 2-amino-3-(pyren-1-yl)propanoic acid, Trp(5-Br), Trp(5-OCH₃), Trp(6-F), Trp(5-OH) or Trp(CHO), 2-aminoadipic acid (2-Aad), 3-aminoadipic acid (3-Aad), propargylglycine (Pra), homopropargylglycine (Hpg), beta-homopropargylglycine (Bpg), 2,3-diaminopropionic acid (Dap), 2,4-diaminobutyric acid (Dab), azidolysine (Lys(N₃)), azido-ornithine (Orn(N₃)), 2-amino-4-azidobutanoic acid Dab(N₃), Dap(N₃), 2-(5′-azidopentyl)alanine, 2-(6′-azidohexyl)alanine, 4-amino-1-carboxymethyl-piperidine (Pip), 4-(2-aminoethyl)-1-carboxymethyl-piperazine (Acp), and tranexamic acid. If not specified as an L- or D-amino acid, an amino acid shall be understood to an L-amino acid.

TABLE 1
List of non-limiting examples of non-proteinogenic amino acids.

| | |
|---|---|
| p-aminomethylaniline-diglycolic acid (pABzA-DIG) | 10-aminodecanoic acid |
| ornithine (Orn) | 2-aminooctanoic acid |
| 3-(1-naphtyl)alanine (Nal) | 2-amino-3-(anthracen-2-yl)propanoic acid |
| 3-(2-naphtyl)alanine (2-Nal) | 2-amino-3-(anthracen-9-yl)propanoic acid |
| α-aminobutyric acid | 2-amino-3-(pyren-1-yl)propanoic acid |
| norvaline | Trp(5-Br), |
| norleucine (Nle) | Trp(5-OCH₃), |
| homonorleucine | Trp(6-F), |
| beta-(1,2,3-triazol-4-yl)-L-alanine | Trp(5-OH) |
| 1,2,4-triazole-3-alanine | Trp(CHO), |
| Phe(4-F), | 2-aminoadipic acid (2-Aad) |
| Phe(4-Cl), | 3-aminoadipic acid (3-Aad) |
| Phe(4-Br), | propargylglycine (Pra) |
| Phe(4-I), | homopropargylglycine (Hpg) |
| Phe(4-NH₂), | beta-homopropargylglycine (Bpg) |
| Phe(4-NO₂), | 2,3-diaminopropionic acid (Dap) |
| homoarginine (hArg) | 2,4-diaminobutyric acid (Dab) |
| 4-(2-aminoethyl)-1-carboxymethyl-piperazine (Acp) | azidolysine (Lys(N₃)) |
| 2-(5′-azidopentyl)alanine, 2-(6′-azidohexyl)alanine | azido-ornithine (Orn(N₃)) |
| 2-amino-4-guanidinobutyric acid (Agb) | amino-4-azidobutanoic acid Dab(N₃) |
| 2-amino-3-guanidinopropionic acid (Agp) | tranexamic acid |
| β-alanine | 4-amino-1-carboxymethyl-piperidine (Pip) |
| 4-aminobutyric acid | NH₂(CH₂)₂O(CH₂)₂C(O)OH |
| 5-aminovaleric acid | NH₂(CH₂)₂[O(CH₂)₂]₂C(O)OH (dPEG2) |
| 6-aminohexanoic acid | NH₂(CH₂)₂[O(CH₂)₂]₃C(O)OH |
| 7-aminoheptanoic acid | NH₂(CH₂)₂[O(CH₂)₂]₄C(O)OH |
| 8-aminooctanoic acid | NH₂(CH₂)₂[O(CH₂)₂]₅C(O)OH |
| 9-aminononanoic acid | NH₂(CH₂)₂[O(CH₂)₂]₆C(O)OH |
| N^{ε},N^{ε},N^{ε}-trimethyl-lysine | any D-amino acid of a proteinogenic amino |
| cysteic acid | acid or any D-amino acid of a non- |
| | proteinogrenic amino acid in this Table |

There is disclosed a compound of Formula Ia:

R^{X}-L-Xaa¹-Gln-Trp-Ala-Val-Xaa²-His-Xaa³-ψ-Xaa⁴-NH₂ (Ia)

wherein:
R^{X} comprises a radionuclide chelator or a trifluoroborate-containing prosthetic group;
L is a linker;
Xaa¹ is D-Phe, Cpa (4-chlorophenylalanine), D-Cpa, Tpi (2,3,4,9-tetrahydro-1H-pyrido[3,4b]indol-3-carboxylic acid), D-Tpi, Nal (naphthylalanine), or D-Nal;

### Xaa² is Gly, N-methyl-Gly or D-Ala;

### Xaa³ is Leu, Pro, D-Pro, or Phe;

Xaa⁴ is Pro, Phe, Tac (thiazolidine-4-carboxylic acid), Nle (norleucine), 4-oxa-L-Pro (oxazolidine-4-carboxylic acid); and
ψ represents a reduced peptide bond between Xaa³ and Xaa⁴.

In some embodiments, Xaa¹ is D-Phe. In other embodiments, Xaa¹ is Cpa. In other embodiments, Xaa¹ is D-Cpa. In other embodiments, Xaa¹ is Tpi. In other embodiments, Xaa¹ is D-Tpi.

In other embodiments, Xaa¹ is Nal. In other embodiments, Xaa¹ is D-Nal. D-Cpa, Tpi, D-Tpi and D-Nal at position Xaa¹ have been shown to retain strong binding affinity for GRPR (e.g. see: Tables 1 and 3 in Cai et al., 1994 Proc. Natl. Acad. Sci. USA 91:12664-12668; RC-3965-II disclosed in Reile et al., 1995 International Journal of Oncology 7:749-754). Since both L-Tpi and D-Tpi retain binding affinity, the L-isomers of D-Nal and D-Cpa would also retain strong binding affinity for GRPR.

In some embodiments, Xaa² is Gly. In other embodiments, Xaa² is N-methyl-Gly. In other embodiments, Xaa² is D-Ala. N-methyl-Gly and D-Ala at position Xaa² have been shown to retain strong binding affinity for GRPR (e.g. see: Table 4 in Horwell et al., 1996 Int. J. Peptide Protein Res. 48:522-531; Table 3 in Lin et al., 1995 European Journal of Pharmacology 284:55-69).

In some embodiments, Xaa³ is Leu. In other embodiments, Xaa³ is Pro. In other embodiments, Xaa³ is D-Pro. In other embodiments, Xaa³ is Phe. D-Pro and Pro at position Xaa³ have been shown to have strong binding affinity for GRPR (e.g. see Table 1 in Leban et al., 1993 Proc. Natl. Acad. Sci. USA 90:1922-1926). Likewise, Phe at position Xaa³ is supported by Phe at this position in ranatensin and litorin, which have very strong binding affinity to the GRPR (Heimbrook et al., 1991 J. Med. Chem. 34:2102-2107; Lin et al., 1995 European Journal of Pharmacology 294:55-69).

In some embodiments, Xaa⁴ is Pro. In other embodiments, Xaa⁴ is Phe. In other embodiments, Xaa⁴ is Tac. In other embodiments, Xaa⁴ is Ne. In other embodiments, Xaa⁴ is 4-oxa-L-Pro. Phe and Ne at position Xaa⁴ have been shown to have strong binding affinity for GRPR (e.g. see Table 1 in Leban et al., 1993 Proc. Natl. Acad. Sci. USA 90:1922-1926). Tac and 4-oxa-L-Pro at position Xaa⁴ would also have strong binding affinity for GRPR based on various peptides with Tac at this position (5, 19-23) and the Examples disclosed herein exemplifying Pro at Xaa⁴.

As represented by the symbol “ψ”, there is a reduced peptide bond between Xaa³ and Xaa⁴, meaning that the main chain amide (e.g. —C(O)NH—) formed between consecutive amino acids in a peptide is replaced by

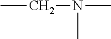

when Xaa⁴ is Pro, Tac or 4-oxa-L-Pro, or is replaced by —CH₂N(R)— when Xaa⁴ is Phe or Nle wherein R is H or C₁-C₅ linear or branched alkyl. In some embodiments, R is H. In other embodiments, R is methyl. In other embodiments, R is C₁-C₅ linear or branched alkyl. In alternative embodiments, R may be methyl, ethyl, n-propyl, i-propyl, sec-propyl, n-butyl, i-butyl, sec-butyl, t-butyl, n-pentyl, i-pentyl, sec-pentyl, t-pentyl, 1,2-dimethylpropyl, or 2-ethylpropyl.

In some embodiments, Xaa⁴ is Phe and ψ is —CH₂NH—. In other embodiments, Xaa⁴ is Phe and ψ is —CH₂N(R)— wherein R is methyl.

In some embodiments, Xaa⁴ is Nle and ψ is —CH₂NH—. In other embodiments, Xaa⁴ is Nle and ψ is —CH₂N(R)— wherein R is methyl.

The linker may be any suitable linker. In some embodiments, the linker is a peptide linker. In some embodiments, the peptide linker is a linear peptide linker. In some embodiments, the peptide linker is a branched peptide linker, where the amino acid residues may be connected through a combination of main chain amide (peptide) bonds and ‘side chain’-to-‘main chain’ or ‘side chain’-to-‘side chain’ bonds. For example, a branched peptide may be connected by one or more of: backbone (main chain) peptide (amide) bonds, ‘main chain’-to-side chain amide bonds (between an amino group and a carboxylic acid group), and/or 1,2,3-triazole linkages (product of a reaction between an azide and an alkyne). In some such embodiments, the peptide linker is (Xaa⁵)₁₋₄, wherein each Xaa⁵ is independently a proteinogenic or non-proteinogenic amino acid residue linked together as a linear or branched peptide linker. In some embodiments, (Xaa⁵)₁₋₄ is a linear peptide linker. In some embodiments, (Xaa⁵)₁₋₄ is a branched peptide linker.

In some embodiments, each Xaa⁵ is independently —N(R^{a})R^{b}C(O)— wherein: R^{a} may be H or methyl; R^{b} may be a 1- to 30-atom alkylenyl, heterolakylenyl, alkenylenyl, heteroalkenylenyl, alkynylenyl, or heteroalkynylenyl, including linear, branched, and/or cyclic (whether aromatic or nonaromatic as well as mono-cyclic, multicyclic or fused cyclic) structures; or N, R^{a} and R^{b} together may form a 5- to 7-atom heteroalkylenyl or heteroalkenylenyl.

In some embodiments, (Xaa⁵)₁₋₄ consists of a single amino acid or residue. In some embodiments, (Xaa⁵)₁₋₄ is a dipeptide, wherein each Xaa⁵ may be the same or different. In some embodiments, (Xaa⁵)₁₋₄ is a tripeptide, wherein each Xaa⁵ may be the same, different or a combination thereof. In some embodiments, (Xaa⁵)₁₋₄ consists of 4 amino acid residues connected by peptide bonds, wherein each Xaa⁵ may be the same, different or a combination thereof. In some embodiments, each Xaa⁵ is independently selected from proteinogenic amino acids and the non-proteinogenic amino acids listed in Table 1, wherein each peptide backbone amino group of the peptide linker is independently optionally methylated. In some embodiments, all peptide backbone amino groups of the peptide linker are methylated. In other embodiments, only one peptide backbone amino group of the peptide linker is methylated. In other embodiments, only two peptide backbone amino groups of the peptide linker are methylated. In other embodiments, no peptide backbone amino groups of the peptide linker are methylated.

In some embodiments, each Xaa⁵ is independently a proteinogenic amino acid residue or is a non-proteinogenic amino acid residue, wherein each peptide backbone amino group is independently optionally methylated, and wherein amino acid residue is independently selected from the group consisting of a proteinogenic amino acid, N^{ε}, N^{ε}, N^{ε}-trimethyl-lysine, 2,3-diaminopropionic acid (Dap), 2,4-diaminobutyric acid (Dab), ornithine (Orn), homoarginine (hArg), 2-amino-4-guanidinobutyric acid (Agb), 2-amino-3-guanidinopropionic acid (Agp), 4-(2-aminoethyl)-1-carboxymethyl-piperazine (Acp), p-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-aminoadipic acid (2-Aad), 3-aminoadipic acid (3-Aad), cysteic acid, tranexamic acid, p-aminomethylaniline-diglycolic acid (pABzA-DIG), 4-amino-1-carboxymethyl-piperidine (Pip), NH₂(CH₂)₂O(CH₂)₂C(O)OH, NH₂(CH₂)₂[O(CH₂)₂]₂C(O)OH (dPEG2), NH₂(CH₂)₂[O(CH₂)₂]₃C(O)OH, NH₂(CH₂)₂[O(CH₂)₂]₄C(O)OH, NH₂(CH₂)₂[O(CH₂)₂]₅C(O)OH, NH₂(CH₂)₂[O(CH₂)₂]₆C(O)OH, and a D- amino acid of any of the foregoing amino acids. In some embodiments, all peptide backbone amino groups of the peptide linker are methylated. In other embodiments, only one peptide backbone amino group of the peptide linker is methylated. In other embodiments, only two peptide backbone amino groups of the peptide linker are methylated. In other embodiments, no peptide backbone amino groups of the peptide linker are methylated.

In some embodiments, the linker is pABzA-DIG. In other embodiments, the linker is Pip. In other embodiments, the linker is dPEG2. In other embodiments, the linker is Acp.

In some embodiments, R^{X} is or comprises a radionuclide chelator. The radionuclide chelator may be any chelator suitable for binding a radiometal, a radionuclide-bound metal, or a radionuclide-bound metal-containing prosthetic group, and which is attached to the linker by forming an amide bond (between an amino group and a carboxylic acid group) or a 1,2,3-triazole (reaction between an azide and an alkyne), or by reaction between a maleimide and a thiol group. Many suitable radionuclide chelators are known, e.g. as summarized in Price and Orvig, Chem. Soc. Rev., 2014, 43, 260-290. In some embodiments, but without limitation, the radionuclide chelator is selected from the group consisting of: DOTA and DOTA derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate; hexamethylpropyleneamineoxime; and hexakis(methoxy isobutyl isonitrile). In some embodiments, the radionuclide chelator is DOTA or a DOTA derivative.

Exemplary non-limiting examples of radionuclide chelators and example radionuclides that may be chelated by these chelators are shown in Table 2. In alternative embodiments, R^{X} is or comprises a radionuclide chelator selected from those listed above or in Table 2. It is noted, however, that one skilled in the art could replace any of the chelators listed herein with another chelator.

TABLE 2
Exemplary chelators and exemplary radionuclide which bind said chelators

| Chelator | Radionuclide |
|---|---|
| 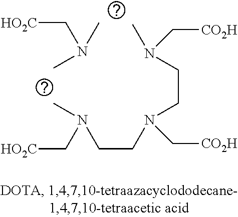 | CU-64/67 Ga-67/68 In-111 Lu-177 Y-86/90 Bi-203/212/213 Pb-212 Ac-225 Gd-159 Yb-175 Ho-166 As-211 Sc-44/47 Pm-149 |
| | Pr-142 |
| | Sn-117m |
| | Sm-153 |
| | Tb-149/152/155/161 |
| | Er-165 |
| | Ra-223/224 |
| | Th-227 |
| 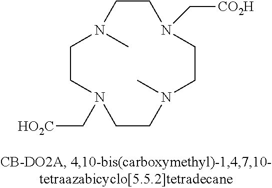 [SMILES: CN1CCN(CC(=O)O)CCN(C)CCN(CC(=O)O)CC1] | Cu-64/67 |
| 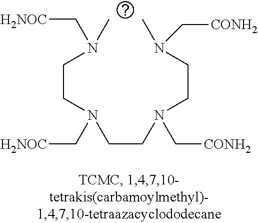 [SMILES: CN(CCN(CCN(CCN(C)CC(N)=O)CC(N)=O)CC(N)=O)CC(N)=O] | Pb-212 |
| 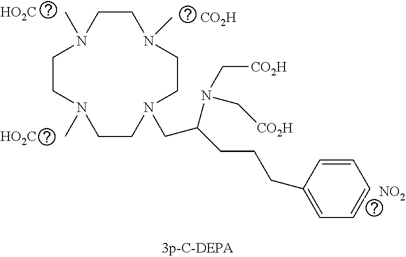 | Bi-212/213 |
| 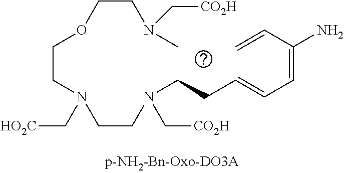 | Cu-64/67 |
| 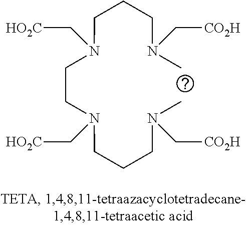 [SMILES: CN(CCCN(CCN(CCCN(C)CC(=O)O)CC(=O)O)CC(=O)O)CC(=O)O] | Cu-64/67 |
| 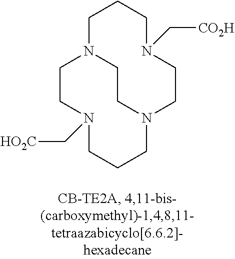 [SMILES: O=C(O)CN1CCCN2CCN(CCCN(CC(=O)O)CC2)CC1] | Cu-64/67 |
| 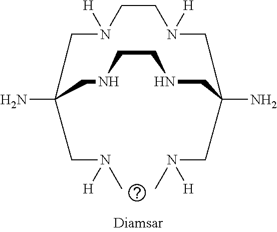 | Cu-64/67 |
| 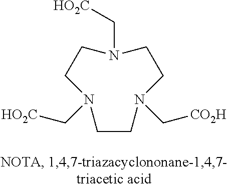 [SMILES: O=C(O)CN1CCN(CC(=O)O)CCN(CC(=O)O)CC1] | Cu-64/67 Ga-68 In-111 Sc-44/47 |
| 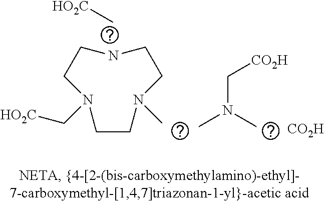 | Cu-64/67 Ga-68 Lu-177 Y-86/90 Bi-213 Pb-212 |
| 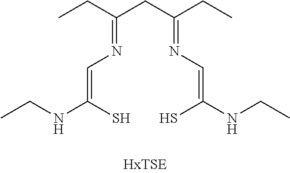 [SMILES: CCN/C(S)=C/N=C(\CC)C/C(CC)=N/C=C(\S)NCC] | Au-198/199 |
| 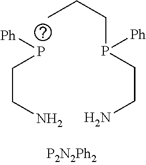 | Rh-105 |
| 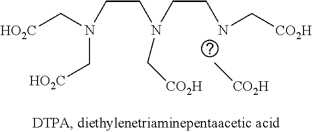 | In-111 Sc-44/47 Lu-177 Y-86/90 Sn-117m Pd-109 |
| 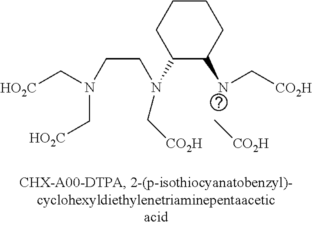 | In-111 Lu-177 Y-86/90 Bi-212/213 |
| 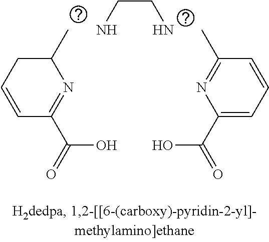 | Cu-64/67 |
| 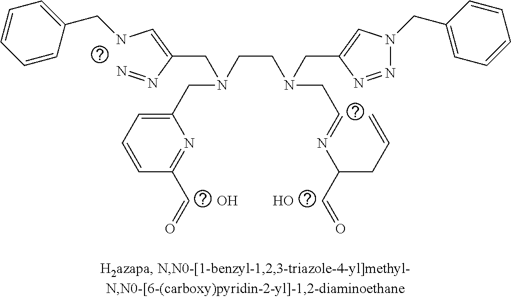 | Cu-64/67 |
| 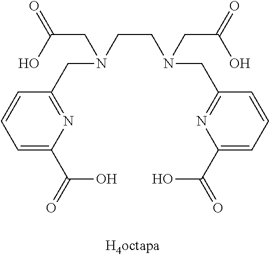 [SMILES: O=C(O)CN(CCN(CC(=O)O)Cc1cccc(C(=O)O)n1)Cc1cccc(C(=O)O)n1] | In-111 Lu-177 Y-86/90 Ac-225 |
| 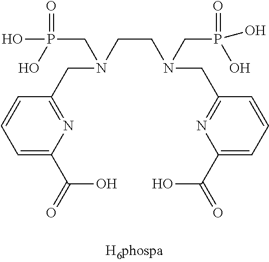 [SMILES: O=C(O)c1cccc(CN(CCN(Cc2cccc(C(=O)O)n2)CP(=O)(O)O)CP(=O)(O)O)n1] | Ac-225 |
| 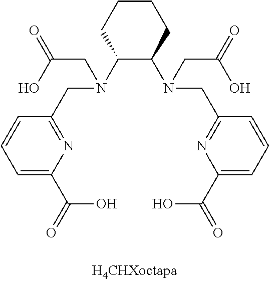 [SMILES: O=C(O)CN(Cc1cccc(C(=O)O)n1)[C@@H]1CCCC[C@H]1N(CC(=O)O)Cc1cccc(C(=O)O)n1] | In-111 Ac-225 |
| 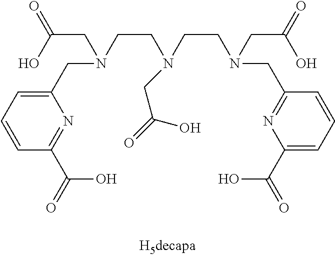 [SMILES: O=C(O)CN(CCN(CC(=O)O)Cc1cccc(C(=O)O)n1)CCN(CC(=O)O)Cc1cccc(C(=O)O)n1] | In-111 Lu-177 Ac-225 |
| 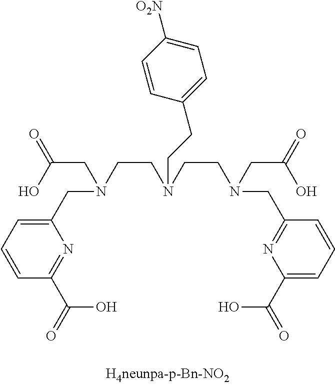 [SMILES: O=C(O)CN(CCN(CCc1ccc([N+](=O)[O-])cc1)CCN(CC(=O)O)Cc1cccc(C(=O)O)n1)Cc1cccc(C(=O)O)n1] | In-111 Lu-177 Ac-225 |
| 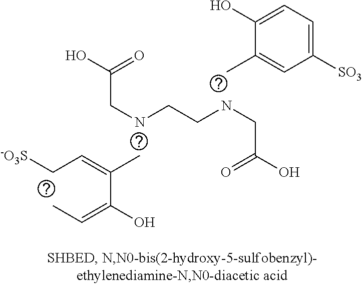 | In-111 Ga-68 |
| 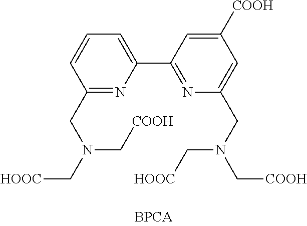 [SMILES: O=C(O)CN(CC(=O)O)Cc1cccc(-c2cc(C(=O)O)cc(CN(CC(=O)O)CC(=O)O)n2)n1] | In-111 |
| 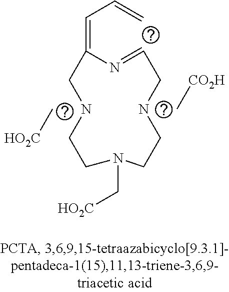 | Cu-64/67 |
| H2-MACROPA (N,N′-bis[(6-carboxy-2-pyridil)methyl]- 4,13-diaza-18-crown-6) | Ac-225 |

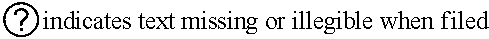

In some embodiments, R^{X} further comprises a radiometal, a radionuclide-bound metal, or a radionuclide-bound metal-containing prosthetic group, and the radiometal, the radionuclide-bound metal, or the radionuclide-bound metal-containing prosthetic group is chelated to the radionuclide-chelator complex. In some embodiments, the radiometal, the radionuclide-bound metal, or the radionuclide-bound metal-containing prosthetic group is: ⁶⁸Ga, ⁶¹Cu, ⁶⁴Cu, ⁶⁷Cu, ⁶⁷Ga, ¹¹¹In, ⁴⁴Sc, ⁸⁶Y, ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, or [¹⁸F]AlF. In other embodiments, the radiometal, the radionuclide-bound metal, or the radionuclide-bound metal-containing prosthetic group is: ⁶⁸Ga, ⁶¹Cu, ⁶⁴Cu, ⁶⁷Cu, ⁶⁷Ga, ¹¹¹In, ⁴⁴Sc, ⁸⁶Y, ¹⁷⁷Lu, ⁹⁰Y, ²²⁵Ac, ²¹³Bi, or ²¹²Bi. In some embodiments, the chelator is a chelator from Table 2 and the chelated radionuclide is a radionuclide indicated in Table 2 as a binder of the chelator.

In some embodiments, the chelator is: DOTA or a derivative thereof, conjugated with ¹⁷⁷Lu, ¹¹¹In, ²¹³Bi ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²¹²Pb, ²²⁵Ac, ²²⁷Th, ²²³Ra, ⁴⁷Sc, ⁶⁴Cu or ⁶⁷Cu; H2-MACROPA conjugated with ²²⁵Ac; Me-3,2-HOPO conjugated with ²²⁷Th; H₄py4pa conjugated with ²²⁵Ac, ²²⁷Th or ¹⁷⁷Lu; H₄pypa conjugated with ¹⁷⁷Lu; NODAGA conjugated with ⁶⁸Ga; DTPA conjugated with ¹¹¹In; or DFO conjugated with ⁸⁹Zr.

In some embodiments, the chelator is TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine), NOTA (1,4,7-triazacyclononane-1,4,7-triaceticacid), TRAP (1,4,7-triazacyclononane-1,4,7-tris[methyl(2-carboxyethyl)phosphinicacid), HBED (N,NO-bis(2-hydroxybenzyl)-ethylenediamine-N,NO-diacetic acid), 2,3-HOPO (3-hydroxypyridin-2-one), PCTA (3,6,9,15-tetraazabicyclo[9.3.1]-pentadeca-1(15),11,13-triene-3,6,9,-triacetic acid), DFO (desferrioxamine), DTPA (diethylenetriaminepentaacetic acid), OCTAPA (N,NO-bis(6-carboxy-2-pyridylmethyl)-ethylenediamine-N,NO-diacetic acid) or another picolinic acid derivative.

In some embodiments, R^{X} is or comprises a chelator for radiolabelling with ^{99m}Tc, ^{94m}Tc, ¹⁸⁶Re, or ¹⁸⁸Re, such as mercaptoacetyl, hydrazinonicotinamide, dimercaptosuccinic acid, 1,2-ethylenediylbis-L-cysteine diethyl ester, methylenediphosphonate, hexamethylpropyleneamineoxime and hexakis(methoxy isobutyl isonitrile), and the like. In some embodiments, In some embodiments, R^{X} is or comprises a chelator, wherein the chelator is mercaptoacetyl, hydrazinonicotinamide, dimercaptosuccinic acid, 1,2-ethylenediylbis-L-cysteine diethyl ester, methylenediphosphonate, hexamethylpropyleneamineoxime or hexakis(methoxy isobutyl isonitrile). In some of these embodiments, the chelator is bound by a radionuclide. In some such embodiments, the radionuclide is ^{99m}Tc, ^{94m}Tc, ¹⁸⁶Re, or ¹⁸⁸Re.

In some embodiments, R^{X} is or comprises a chelator that can bind ¹⁸F-aluminum fluoride ([¹⁸F]AlF), such as 1,4,7-triazacyclononane-1,4-diacetate (NODA) and the like. In some embodiments, the chelator is NODA. In some embodiments, the chelator is bound by [¹⁸F]AlF.

In some embodiments, R^{X} is or comprises a chelator that can bind ⁷²As or ⁷⁷As, such as a trithiol chelate and the like. In some embodiments, the chelator is a trithiol chelate. In some embodiments, the chelator is conjugated to ⁷²As. In some embodiments, the chelator is conjugated to ⁷⁷As.

In certain embodiments, R^{X} is or comprises a prosthetic group containing a trifluoroborate (BF₃), capable of ¹⁸F/¹⁹F exchange radiolabeling. In some of these embodiments, R^{X} is R¹R²BF₃, wherein: R¹ is

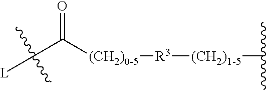

wherein L is the linker, and R³ is absent,

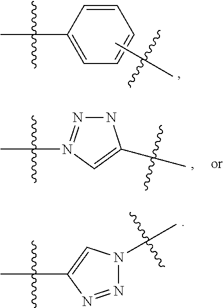

The group —R²BF₃ may be one of those listed in Table 3 (below) or Table 4 (below), or is

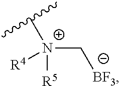

wherein R⁴ and R⁵ are independently C₁-C₅ linear or branched alkyl groups.

In certain embodiments, R^{X} is or comprises more than one (e.g. 2, 3 or 4) prosthetic groups each containing a trifluoroborate (BF₃) capable of ¹⁸F/¹⁹F exchange radiolabeling. In some of these embodiments, R^{X} comprises more than one R¹R²BF₃, wherein: each R¹ is independently

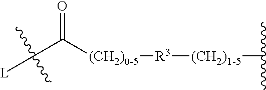

wherein L is the linker, and each R³ is independently absent,

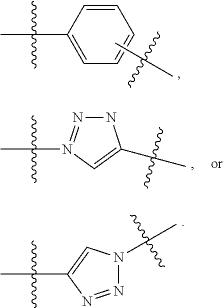

Each —R²BF₃ may independently be one of those listed in Table 3 (below) or Table 4 (below), or

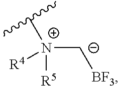

wherein each R⁴ is independently a C₁-C₅ linear or branched alkyl group and each R⁵ is independently a C₁-C₅ linear or branched alkyl group. In some embodiments, R^{X} is or comprises exactly two R¹R²BF₃ groups attached to the linker. In some such embodiments, the linker is a branched peptide linker wherein each R¹R²BF₃ group is attached to the linker by forming an amide bond to an amino group of the linker. For example, when the linker is (Xaa⁵)₁₋₄, an R¹R²BF₃ group may bond to the N-terminus of the N-terminal Xaa⁵, and/or R¹R²BF₃ groups may bond to any other free amino group of Xaa⁵. Non-limiting examples of amino acid residues with a side chain capable of forming an amide with an R¹R²BF₃ group include Lys, Orn, Dab, Dap, Arg, homo-Arg, and the like. In some embodiments, R¹R²BF₃ bonds to the N-terminus of the N-terminal Xaa⁵. In some embodiments, a first R¹R²BF₃ group may bond to the N-terminus of the N-terminal Xaa⁵ and a second R¹R²BF₃ group may bond to a side chain functional group (e.g. an amino group) of an Xaa⁵.

Alternatively, each of two R¹R²BF₃ groups may bond to different Xaa⁵ side chains or other functional groups.

For Tables 3 and 4 below, each R in the pyridine substituted with —OR, —SR, —NR—, —NHR or —NR₂ groups is independently a C₁-C₅ linear or branched alkyl. In some embodiments, the —R²BF₃ group(s) is/are selected from those listed in Table 3. In some embodiments, the —R²BF₃ group(s) is/are selected from those listed in Table 4. The trifluoroborate-containing prosthetic group(s) may comprise ¹⁸F. In some embodiments, one fluorine in —R²BF₃ is ¹⁸F. In some embodiments, all three fluorines in —R²BF₃ are ¹⁸F. In some embodiments, all three fluorines in —R²BF₃ are ¹⁹F.

TABLE 3
Exemplary -R²BF₃ groups.

| | | | |
|---|---|---|---|
| 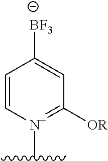 | 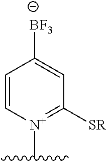 | 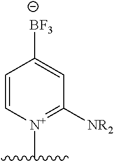 | 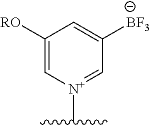 |
| 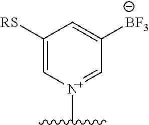 | 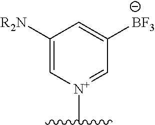 | 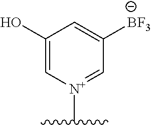 | 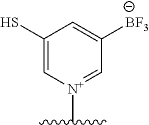 |
| 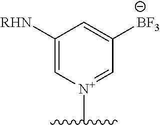 | 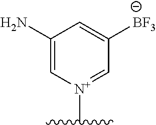 | 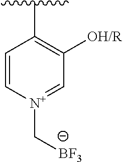 | 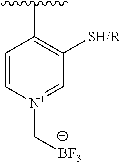 |
| 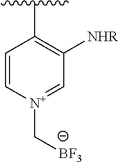 | 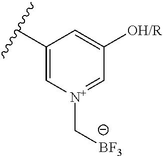 | 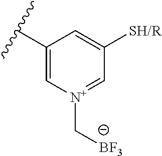 | 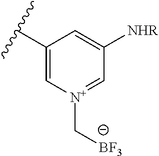 |
| 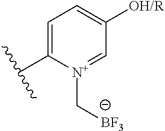 | 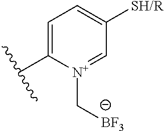 | 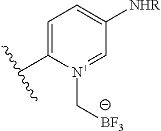 | 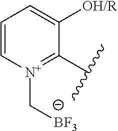 |
| 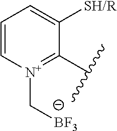 | 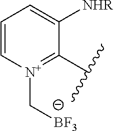 | 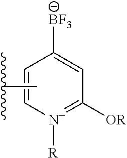 | 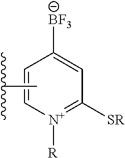 |
| 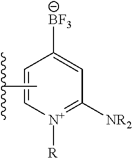 | 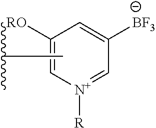 | 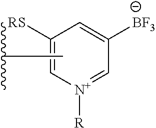 | 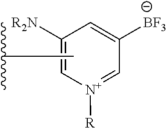 |
| 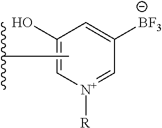 | 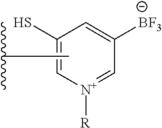 | 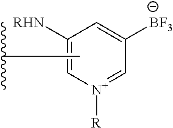 | 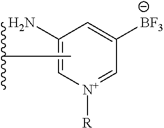 |
| 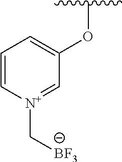 | 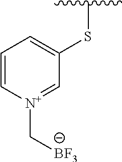 | 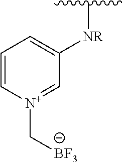 | 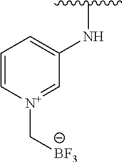 |
| 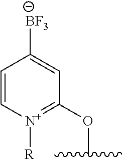 | 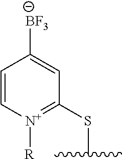 | 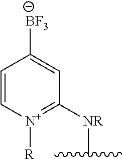 | 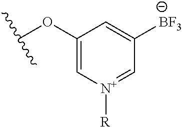 |
| 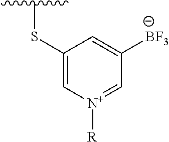 | 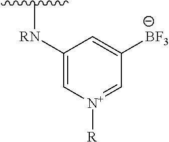 | 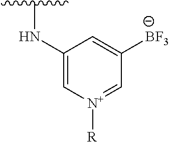 | 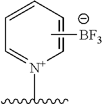 |

TABLE 4
Exemplary -R²BF₃ groups.

| | | | |
|---|---|---|---|
| 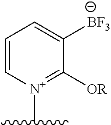 | 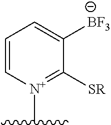 | 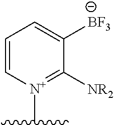 | 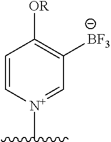 |
| 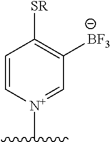 | 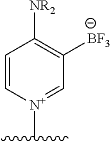 | 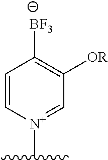 | 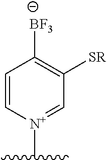 |
| 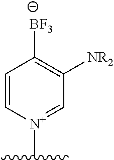 | 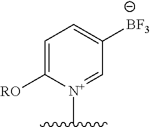 | 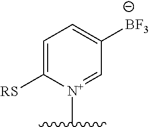 | 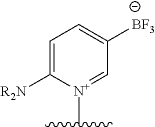 |
| 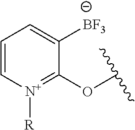 | 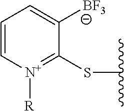 | 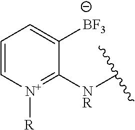 | 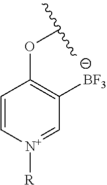 |
| 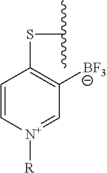 | 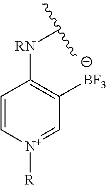 | 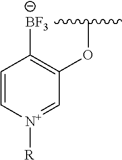 | 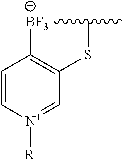 |
| 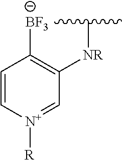 | 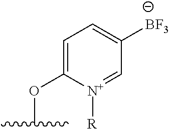 | 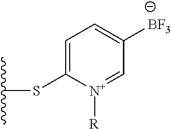 | 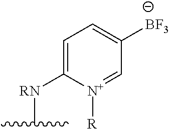 |
| 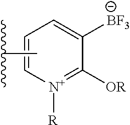 | 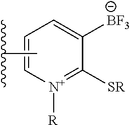 | 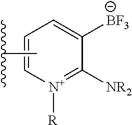 | 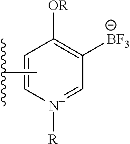 |
| 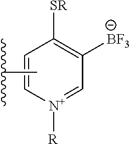 | 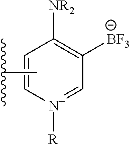 | 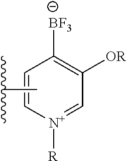 | 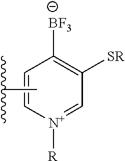 |
| 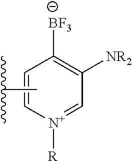 | 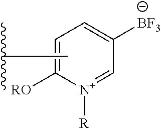 | 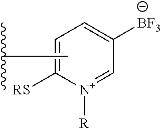 | 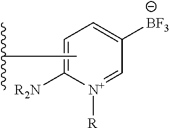 |
| 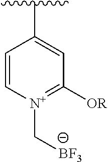 | 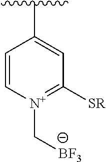 | 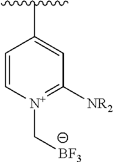 | 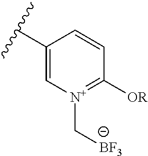 |
| 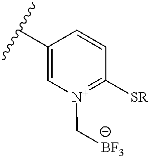 | 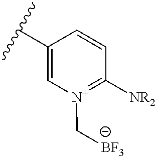 | 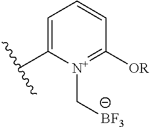 | 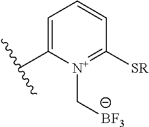 |
| 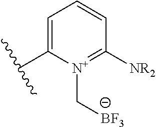 | 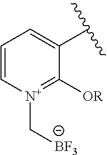 | 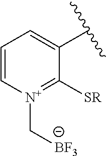 | 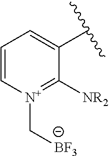 |
| 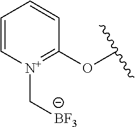 | 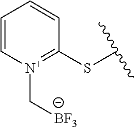 | 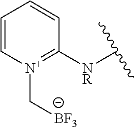 | 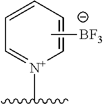 |

In some embodiments, each —R²BF₃ may independently form

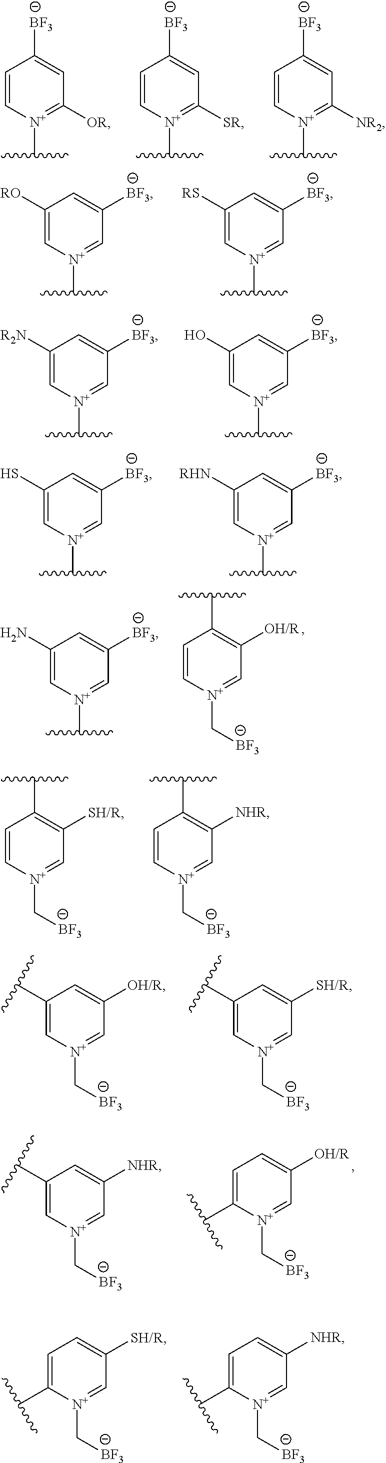

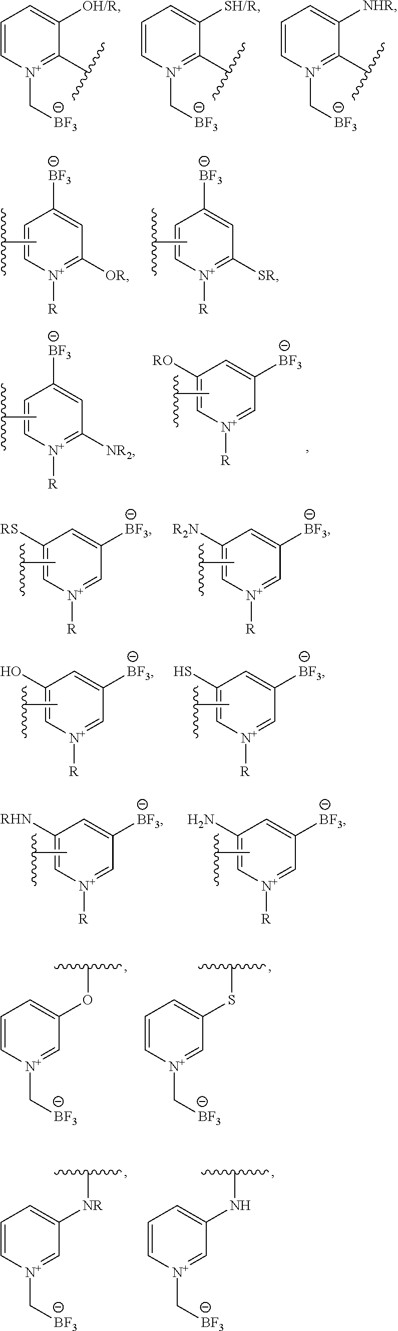

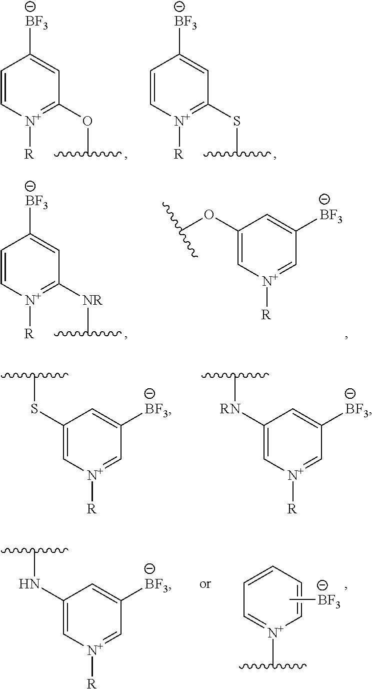

in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. The trifluoroborate-containing prosthetic group(s) may comprise ¹⁸F. In some embodiments, one fluorine is —R²BF₃ is ¹⁸F. In some embodiments, all three fluorines in —R²BF₃ are ¹⁸F. In some embodiments, all three fluorines in —R²BF₃ are ¹⁹F.

In some embodiments, each —R²BF₃ may independently form

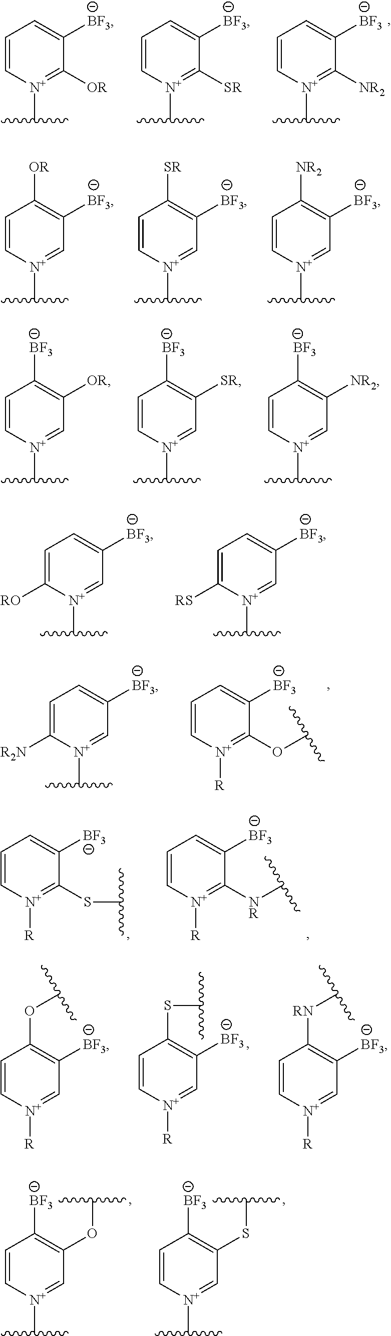

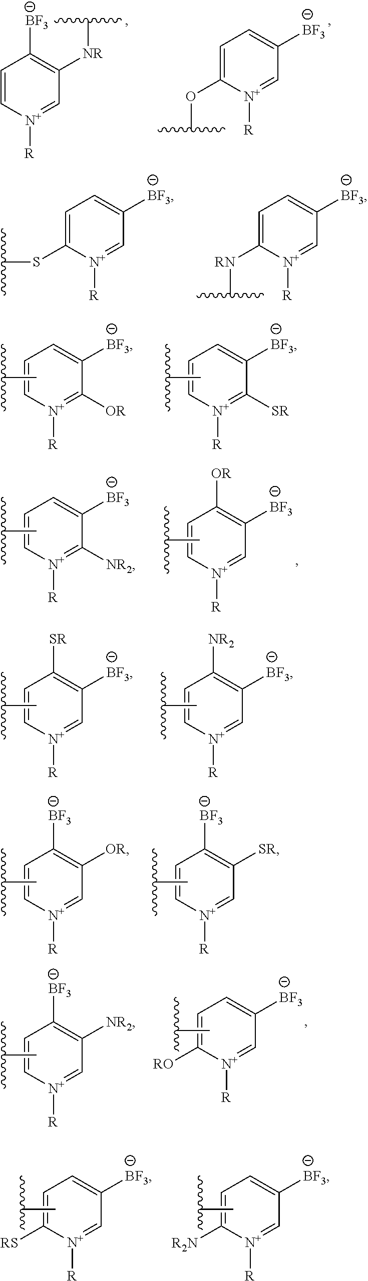

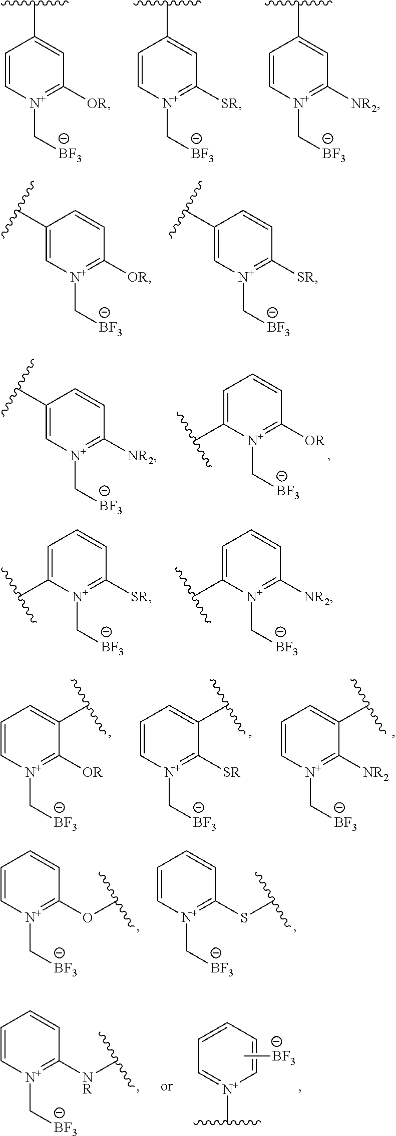

in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. In some embodiments, —R²BF₃ is

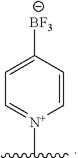

In some embodiments, all three fluorines in —R²BF₃ are ¹⁸F. In some embodiments, one fluorine in —R²BF₃ is ¹⁸F. In some embodiments, all three fluorines in —R²BF₃ are ¹⁹F.

In some embodiments, each —R²BF₃ is independently

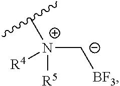

wherein R⁴ and R⁵ are independently C₁-C₅ linear or branched alkyl groups. In some embodiments, R⁴ is methyl. In some embodiments, R⁴ is ethyl. In some embodiments, R⁴ is propyl. In some embodiments, R⁴ is isopropyl. In some embodiments, R⁴ is butyl. In some embodiments, R⁴ is n-butyl. In some embodiments, R⁴ is pentyl. In some embodiments, R⁵ is methyl. In some embodiments, R⁵ is ethyl. In some embodiments, R⁵ is propyl. In some embodiments, R⁵ is isopropyl. In some embodiments, R⁵ is butyl. In some embodiments, R⁵ is n-butyl. In some embodiments, R⁵ is pentyl. In some embodiments, R⁴ and R⁵ are both methyl. The trifluoroborate-containing prosthetic group may comprise ¹⁸F. In some embodiments, one fluorine in —R²BF₃ is ¹⁸F. In some embodiments, all three fluorines in —R²BF₃ are ¹⁸F. In some embodiments, all three fluorines in-R²BF₃ are ¹⁹F.

In certain embodiments, the compound is conjugated with a radionuclide for positron emission tomography (PET) or single photon emission computed tomography (SPECT) imaging of GRPR expressing tumors, wherein the compound is conjugated with a radionuclide that is a positron emitter or a gamma emitter. Without limitation, the positron or gamma emitting radionuclide is ⁶⁸Ga, ⁶⁷Ga, ⁶¹Cu, ⁶⁴Cu ⁶⁷Ga, ^{99m}Tc, ^{110m}In, ¹¹¹In, ⁴⁴Sc, ⁸⁶Y, ⁸⁹Zr, ⁹⁰Nb, ¹⁵²Tb, ¹⁵⁵Tb, ¹⁸F, ¹³¹I, ¹²³I, ¹²⁴I and ⁷²As.

In certain embodiments the compound is conjugated with a radionuclide that is used for therapy. This includes radioisotopes such as ¹⁶⁵Er, ²¹²Bi, ²¹¹At, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ²⁰³Pb, ²¹²Pb, ⁴⁴Sc, ⁴⁷Sc, ⁹⁰Y, ²²⁵Ac, ^{117m}Sn, ¹⁵³Sm, ¹⁴⁹Tb, ¹⁶¹Tb, ¹⁶⁵Er, ²¹³Bi, ²²⁴Ra, ²¹²Bi ²¹²Pb, ²²⁵Ac, ²²⁷Th, ²²³Ra, ⁴⁷Sc, ⁷⁷As, ⁶⁴Cu or ⁶⁷Cu.

The compound may have the following chemical structure or be a salt or solvate thereof, optionally chelated with radionuclide X:

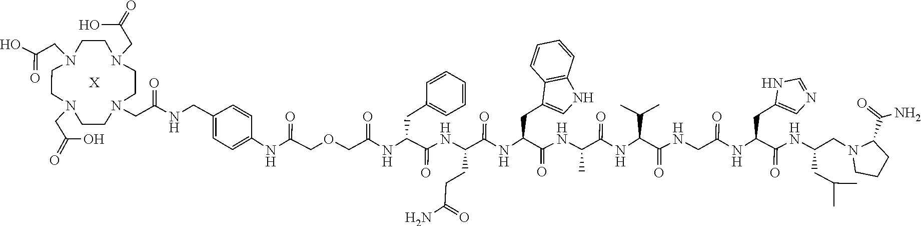

(ProBOMB1). In alternative embodiments, X is ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, 86Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²¹²Pb, ²²⁵Ac, ²²⁷Th, ²²³Ra, ⁴⁷Sc, ⁶⁴Cu or ⁶⁷Cu. In some embodiments, X is ⁶⁸Ga. In some embodiments, X is ⁶⁴Cu. In some embodiments, X is ⁶⁷Cu. In some embodiments, X is ⁶⁷Ga. In some embodiments, X is ¹¹¹In. In some embodiments, X is ¹⁷⁷Lu. In some embodiments, X is ⁹⁰Y In some embodiments, X is ²²⁵Ac.

The compound may have the following chemical structure or be a salt or solvate thereof, optionally chelated with radionuclide X:

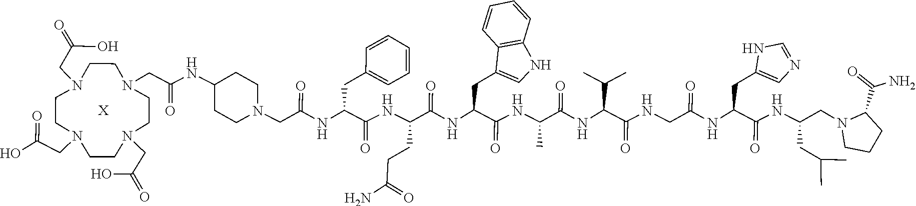

(ProBOMB2). In alternative embodiments, X is ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²¹²Pb, ²²⁵Ac, ²²⁷Th, ²²³Ra, ⁴⁷Sc, ⁶⁴Cu or ⁶⁷Cu. In some embodiments, X is ⁶⁸Ga. In some embodiments, X is ⁶⁴Cu. In some embodiments, X is ⁶⁷Cu. In some embodiments, X is ⁶⁷Ga. In some embodiments, X is ¹¹¹In. In some embodiments, X is ¹⁷⁷Lu. In some embodiments, X is ⁹⁰Y In some embodiments, X is ²²⁵Ac.

There is also disclosed a compound/composition of Formula Ib:

(Radionuclide-chelator complex)-(Linker)-AA1-Gln-Trp-Ala-Val-AA2-His-AA3-ψ-AA4-NH₂ (1b)

In some embodiments, the compound/composition of Formula 1b is ⁶⁸Ga-ProBOMB1 (⁶⁸Ga-DOTA-pABzA-DIG-D-Phe-Gln-Trp-Ala-Val-Gly-His-Leu-y-Pro-NH₂; see above for structure of ProBOMB1). This compound is useful for in-vivo PET imaging of tissues expressing the GRPR. As such, this and other compounds/compositions disclosed herein are useful for the diagnosis and detection of diseases or disorders characterized by aberrant/ectopic expression of the GRPR, including but not limited to various forms of cancer.

In some embodiments, the radionuclide ⁶⁸Ga in ⁶⁸Ga-ProBOMB1 may be replaced by other trivalent radiometals such as ⁹⁰Y or ¹⁷⁷Lu, which can form stable complexes with DOTA. These novel compositions (representing a theranostic pair with ⁶⁸Ga-ProBOMB1) comprise new radiotherapeutic agents for treatment of disorder or diseases (including but not limited to cancer) characterized by aberrant/ectopic expression of the GRPR.

In some embodiments, the chelator DOTA in ⁶⁸Ga-ProBOMB1 may be substituted/replaced by other suitable chelators including but not limited to other radiometal chelators such as DOTAGA, NOTA, or NOTAGA, or trifluoroborate for radiolabeling with fluorine-18 (¹⁸F).

In some embodiments, the linker p-aminomethylaniline-diglycolic acid (pABzA-DIG) in ⁶⁸Ga-ProBOMB1 may be substituted/replaced by other suitable linkers including but not limited to Pip (4-amino-(1-carboxymethyl)piperidine) or dPEG2 (9-amino-4,7-dioxanonanoic acid).

In some embodiments, the AA1 D-Phe in ⁶⁸Ga-ProBOMB1 may be substituted/replaced by other suitable amino acids including but not limited to D-Cpa (4-chlorophenylalanine), Cpa, Tpi (2,3,4,9-tetrahydro-1H-pyrido[3,4b]indol-3-carboxylic acid), D-Tpi, Nal, or D-Nal.

In some embodiments, the AA2 Gly in ⁶⁸Ga-ProBOMB1 may be substituted/replaced by other suitable amino acids including but not limited to N-methyl-Gly or D-Ala.

In some embodiments, the AA3 Leu in ⁶⁸Ga-ProBOMB1 may be substituted/replaced by other suitable amino acids including but not limited to D-Pro, Pro or Phe.

In some embodiments, the AA4 Pro in ⁶⁸Ga-ProBOMB1 may be substituted/replaced by other suitable amino acids including but not limited to Phe, Tac (thiazolidine-4-carboxylic acid) or N-methyl-Leu.

Referring to compounds of Formula 1a and Formula 1b, when the radiolabeling group (i.e. R^{X} in Formula 1a, or the radionuclide-chelator complex or trifluoroborate of Formula 1b) comprises or is conjugated to a diagnostic radionuclide, there is disclosed use of certain embodiments of a compound as disclosed herein (i.e. a compound of Formula Ia, Formula 1b, or a salt or solvate thereof) for preparation of a radiolabelled tracer for imaging GRPR-expressing tissues in a subject. There is also disclosed a method of imaging GRPR-expressing tissues in a subject, in which the method comprises: administering to the subject a composition comprising certain embodiments of the compound (i.e. of Formula 1a, Formula 1b, or a salt or solvate thereof) and a pharmaceutically acceptable excipient; and imaging tissue of the subject, e.g. using PET or SPECT. When the tissue is a diseased tissue (e.g. a GRPR-expressing cancer), GRPR-targeted treatment may then be selected for treating the subject.

Referring again to compounds of Formula Ia and Formula 1b, when the radiolabeling group (i.e. R^{X} in Formula Ia, or the radionuclide-chelator complex or trifluoroborate of Formula 1b) comprises a therapeutic radionuclide, there is disclosed use of certain embodiments of the compound (or a pharmaceutical composition thereof) for the treatment of GRPR-expressing conditions or diseases (e.g. cancer and the like) in a subject. Accordingly, there is provided use of a compound disclosed herein (i.e. of Formula 1a, Formula 1b, or a salt or solvate thereof) in preparation of a medicament for treating a GRPR-expressing condition or disease in a subject. There is also provided a method of treating GRPR-expressing disease in a subject, in which the method comprises: administering to the subject a composition comprising the compound (i.e. of Formula 1a, Formula 1b, or a salt or solvate thereof) and a pharmaceutically acceptable excipient. For example, but without limitation, the disease may be a GRPR-expressing cancer.

Aberrant or ectopic GRPR expression has been detected in various conditions and diseases, including psychiatric/neurological disorders, inflammatory disease, and cancer (5, 19-23, and 39-43). Accordingly, without limitation, the GRPR-expressing condition or disease may be psychiatric disorder, neurological disorder, inflammatory disease, prostate cancer, lung cancer, head and neck cancer, colon cancer, kidney cancer, ovarian cancer, liver cancer, pancreatic cancer, breast cancer, glioma or neuroblastoma. In some embodiments, the cancer is prostate cancer.

The compounds presented herein incorporate peptides, which may be synthesized by any of a variety of methods established in the art. This includes but is not limited to liquid-phase as well as solid-phase peptide synthesis using methods employing 9-fluorenylmethoxycarbonyl (Fmoc) and/or t-butyloxycarbonyl (Boc) chemistries, and/or other synthetic approaches.

Solid-phase peptide synthesis methods and technology are well-established in the art. For example, peptides may be synthesized by sequential incorporation of the amino acid residues of interest one at a time. In such methods, peptide synthesis is typically initiated by attaching the C-terminal amino acid of the peptide of interest to a suitable resin. Prior to this, reactive side chain and alpha amino groups of the amino acids are protected from reaction by suitable protecting groups, allowing only the alpha carboxyl group to react with a functional group such as an amine group, a hydroxyl group, or an alkyl halide group on the solid support. Following coupling of the C-terminal amino acid to the support, the protecting group on the side chain and/or the alpha amino group of the amino acid is selectively removed, allowing the coupling of the next amino acid of interest. This process is repeated until the desired peptide is fully synthesized, at which point the peptide can be cleaved from the support and purified. A non-limiting example of an instrument for solid-phase peptide synthesis is the Aapptec Endeavor 90 peptide synthesizer.

To allow coupling of additional amino acids, Fmoc protecting groups may be removed from the amino acid on the solid support, e.g. under mild basic conditions, such as piperidine (20-50% v/v) in DMF. The amino acid to be added must also have been activated for coupling (e.g. at the alpha carboxylate). Non-limiting examples of activating reagents include without limitation 2-(1H-benzotriazol-1-yl)-1,1,3,3-tetramethyluronium hexafluorophosphate (HBTU), 2-(1H-benzotriazol-1-yl)-1,1,3,3-tetramethyluronium tetrafluoroborate (TBTU), 2-(7-Aza-1H-benzotriazole-1-yl)-1,1,3,3-tetramethyluronium hexafluorophosphate (HATU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP). Racemization is minimized by using triazoles, such as 1-hydroxy-benzotriazole (HOBt) and 1-hydroxy-7-aza-benzotriazole (HOAt). Coupling may be performed in the presence of a suitable base, such as N,N-diisopropylethylamine (DIPEA/DIEA) and the like.

Apart from forming typical peptide bonds to elongate a peptide, peptides may be elongated in a branched fashion by attaching to side chain functional groups (e.g. carboxylic acid groups or amino groups), either: side chain to side chain; or side chain to backbone amino or carboxylate. Coupling to amino acid side chains may be performed by any known method, and may be performed on-resin or off-resin. Non-limiting examples include: forming an amide between an amino acid side chain containing a carboxyl group (e.g. Asp, D-Asp, Glu, D-Glu, and the like) and an amino acid side chain containing an amino group (e.g. Lys, D-Lys, Orn, D-Orn, Dab, D-Dab, Dap, D-Dap, and the like) or the peptide N-terminus; forming an amide between an amino acid side chain containing an amino group (e.g. Lys, D-Lys, Orn, D-Orn, Dab, D-Dab, Dap, D-Dap, and the like) and either an amino acid side chain containing a carboxyl group (e.g. Asp, D-Asp, Glu, D-Glu, and the like) or the peptide C-terminus; and forming a 1, 2, 3-triazole via click chemistry between an amino acid side chain containing an azide group (e.g. Lys(N₃), D-Lys(N₃), and the like) and an alkyne group (e.g. Pra, D-Pra, and the like). The protecting groups on the appropriate functional groups must be selectively removed before amide bond formation, whereas the reaction between an alkyne and an azido groups via the click reaction to form an 1,2,3-triazole does not require selective deprotection. Non-limiting examples of selectively removable protecting groups include 2-phenylisopropyl esters (O-2-PhiPr) (e.g. on Asp/Glu) as well as 4-methyltrityl (Mtt), allyloxycarbonyl (alloc), 1-(4,4-dimethyl-2,6-dioxocyclohex-1-ylidene))ethyl (Dde), and 1-(4,4-dimethyl-2,6-dioxocyclohex-1-ylidene)-3-methylbutyl (ivDde) (e.g. on Lys/Orn/Dab/Dap). O-2-PhiPr and Mtt protecting groups can be selectively deprotected under mild acidic conditions, such as 2.5% trifluoroacetic acid (TFA) in DCM. Alloc protecting groups can be selectively deprotected using tetrakis(triphenylphosphine)palladium(0) and phenyl silane in DCM. Dde and ivDde protecting groups can be selectively deprotected using 2-5% of hydrazine in DMF.

Deprotected side chains of Asp/Glu (L- or D-forms) and Lys/Orn/Dab/Dap (L- or D-forms) can then be coupled, e.g. by using the coupling reaction conditions described above. The above provides means for including multiple BF₃ groups.

Peptide backbone amides may be N-methylated (i.e. alpha amino methylated). This may be achieved by directly using Fmoc-N-methylated amino acids during peptide synthesis. Alternatively, N-methylation under Mitsunobu conditions may be performed. First, a free primary amine group is protected using a solution of 4-nitrobenzenesulfonyl chloride (Ns-CI) and 2,4,6-trimethylpyridine (collidine) in NMP. N-methylation may then be achieved in the presence of triphenylphosphine, diisopropyl azodicarboxylate (DIAD) and methanol. Subsequently, N-deprotection may be performed using mercaptoethanol and 1,8-diazabicyclo[5.4.0]undec-7-ene (DBU) in NMP. For coupling protected amino acids to N-methylated alpha amino groups, HATU, HOAt and DIEA may be used.

Non-peptide moieties (e.g. radiolabeling groups and/or linkers) may be coupled to the peptide N-terminus while the peptide is attached to the solid support. This is facile when the non-peptide moiety comprises an activated carboxylate (and protected groups if necessary) so that coupling can be performed on resin. For example, but without limitation, a bifunctional chelator, such as 1,4,7,10-tetraazacyclododecane-1,4,7,10-tetraacetic acid (DOTA) tris(tert-butyl ester) may be activated in the presence of N-hydroxysuccinimide (NHS) and N,N′-dicyclohexylcarbodiimide (DCC) for coupling to a peptide. Alternatively, a non-peptide moiety may be incorporated into the compound via a copper-catalyzed click reaction under either liquid or solid phase conditions. Copper-catalyzed click reactions are well established in the art. For example, 2-azidoacetic acid is first activated by NHS and DCC and coupled to a peptide. Then, an alkyne-containing non-peptide moeity may be clicked to the azide-containing peptide in the presence of Cu²⁺ and sodium ascorbate in water and organic solvent, such as acetonitrile (ACN) and DMF and the like.

The synthesis of radiometal chelators is well-known and many chelators are commercially available (e.g. from Sigma-Aldrich™/Milipore Sigma™ and others). Protocols for conjugation of radiometals to the chelators is also well known (e.g. see Example 1, below).

The synthesis of the R¹R²BF₃ component of the compounds can be achieved following previously reported procedures (Liu et al. Angew Chem Int Ed 2014 53:11876-11880; Liu et al. J Nucl Med 2015 55:1499-1505; Liu et al. Nat Protoc 2015 10:1423-1432; Kuo et al. J Nucl Med, 2019 60:1160-1166; each of which is incorporated by reference in its entirety). Generally, the BF₃-containing motif can be coupled to the linker via click chemistry by forming a 1,2,3-triazole ring between a BF₃-containing azido (or alkynyl) group and an alkynyl (or azido) group on the linker, or by forming an amide linkage between a BF₃-containing carboxylate and an amino group on the linker. To make the BF₃-containing azide, alkyne or carboxylate, a boronic acid ester-containing azide, alkyne or carboxylate is first prepared following by the conversion of the boronic acid ester to BF₃ in a mixture of HCl, DMF and KHF₂. For alkyl BF₃, the boronic acid ester-containing azide, alkyne or carboxylate can be prepared by coupling boronic acid ester-containing alkyl halide (such as iodomethylboronic acid pinacol ester) with an amine-containing azide, alkyne orcarboxylate (such as N,N-dimethylpropargylamine). For aryl BF₃, the boronic acid ester can be prepared via Suzuki coupling using aryl halide (iodine or bromide) and bis(pinacolato)diboron.

¹⁸F-Fluorination of the BF₃-containing compounds via ¹⁸F-¹⁹F isotope exchange reaction can be achieved following previously published procedures (Liu et al. Nat Protoc 2015 10:1423-1432, incorporated by reference in its entirety). Generally, ˜100 nmol of the BF₃-containing compound is dissolved in a mixture of 15 μl of pyridazine-HCl buffer (pH=2.0-2.5, 1 M), 15 μl of DMF and 1 μl of a 7.5 mM KHF₂ aqueous solution. ¹⁸F-Fluoride solution (in saline, 60 μl) is added to the reaction mixture, and the resulting solution is heated at 80° C. for 20 min. At the end of the reaction, the desired product can be purified by solid phase extraction or by reversed high performance liquid chromatography (HPLC) using a mixture of water and acetonitrile as the mobile phase.

When the peptide has been fully synthesized on the solid support, the desired peptide may be cleaved from the solid support using suitable reagents, such as TFA, tri-isopropylsilane (TIS) and water. Side chain protecting groups, such as Boc, pentamethyldihydrobenzofuran-5-sulfonyl (Pbf), trityl (Trt) and tert-butyl (tBu) are simultaneously removed (i.e. deprotection). The crude peptide may be precipitated and collected from the solution by adding cold ether followed by centrifugation. Purification and characterization of the peptides may be performed by standard separation techniques, such as high performance liquid chromatography (HPLC) based on the size, charge and polarity of the peptides. The identity of the purified peptides may be confirmed by mass spectrometry or other similar approaches.

A synthetic scheme for exemplary compounds ProBOMB1 and ProBOMB2 and conjugation with ⁶⁸Ga and ¹⁷Lu is described in the following Examples. The following Examples show that compounds of the invention can have nanomolar affinity for GRPR and high stability in vivo, and can generate high-contrast images (e.g. PET) with good tumor uptake and extremely low pancreas uptake, which is an advantage over prior art tracers derived from BBN.

The present invention will be further illustrated in the following examples.

### Example 1: ProBOMB1

1.1 Materials and Methods

ProBOMB1 (DOTA-pABzA-DIG-D-Phe-Gln-Trp-Ala-Val-Gly-His-Leu-ψ(CH₂N)-Pro-NH₂) was synthesized by solid-phase peptide synthesis. The polyaminocarboxylate chelator 1,4,7,10-tetraazacyclododecane-1,4,7,10-tetraacetic acid (DOTA) was coupled to the N-terminus and separated from the GRPR-targeting sequence by a p-aminomethylaniline-diglycolic acid (pABzA-DIG) linker. Binding affinity to GRPR was determined using a cell-based competition assay, while agonist/antagonist property was determined with a calcium efflux assay.

For ⁶⁸Ga-ProBOMB1, ProBOMB1 was radiolabeled with ⁶⁸GaCl₃. For ¹⁷⁷Lu-ProBOMB1, ProBOMB1 was radiolabeled with ¹⁷⁷LuCl₃. PET imaging and biodistribution studies were performed in male immunocompromised mice bearing PC-3 prostate cancer xenografts. Blocking experiments were performed with co-injection of [D-Phe6, Leu-NHEt13, des-Met14]Bombesin(6-14). Dosimetry calculations were performed with OLINDA software.

All reagents and solvents were purchased from commercial sources and used without further purification. [D-Phe⁶, Leu-NHEt¹³, des-Met¹⁴]Bombesin(6-14) and Bombesin were purchased from Bachem and Anaspec, respectively. Other peptides were synthesized on an AAPPTec Endeavor 90 peptide synthesizer. High-performance liquid chromatography (HPLC) was performed on an Agilent 1260 infinity system (model 1200 quaternary pump, model 1200 UV absorbance detector set at 220 nm, Bioscan Nal scintillation detector). HPLC columns used were a semi-preparative column (Luna C18, 5μ, 250×10 mm) and an analytical column (Luna, C18, 5μ, 250×4.6 mm) from Phenomenex. Mass analyses were performed using an AB SCIEX4000 QTRAP mass spectrometer with an ESI ion source. ⁶⁸Ga was eluted from an iThemba Labs generator and purified according to previously published procedures using a DGA resin column from Eichrom Technologies LLC (24). Radioactivity of ⁶⁸Ga-labeled peptides was measured using a Capintec CRC-25R/W dose calibrator, and the radioactivity in tissues collected from biodistribution studies were counted using a Perkin Elmer Wizard2 2480 gamma counter.

1.1.1 Chemistry and Radiolabeling.

The synthesis procedures for radiolabeling precursors and nonradioactive standards are shown below.

1.1.1.1 ⁶⁸Ga-ProBOMB1 and ⁶⁸Ga-NeoBOMB1.

Purified ⁶⁸GaCl₃ (289-589 MBq, in 0.6 mL water) was added to 0.6 mL of HEPES buffer (2 M, pH 5.3) containing ProBOMB1 or NeoBOMB1 (25 μg). The mixture was heated by microwave oven (Danby DMW7700WDB; power setting 2; 1 min).

HPLC purification was used to separate ⁶⁸Ga-labeled product from the unlabeled precursor (semi-preparative column; 23% acetonitrile and 0.1% TFA in water for ⁶⁸Ga-ProBOMB1; 35% acetonitrile and 0.1% HCOOH in water for ⁶⁸Ga-NeoBOMB1; flow rate: 4.5 mL/min). Retention times: 23.7 min (⁶⁸Ga-ProBOMB1); 11.0 min (⁶⁸Ga-NeoBOMB1). The fraction containing ⁶⁸Ga-ProBOMB1 or ⁶⁸Ga-NeoBOMB1 was collected, diluted with water (50 mL), and passed through a C18 Sep-Pak cartridge. The ⁶⁸Ga-ProBOMB1 or ⁶⁸Ga-NeoBOMB1 trapped on the cartridge was eluted off with ethanol (0.4 mL) and diluted with phosphate-buffered saline (PBS). Quality control was performed using the analytical column: 24% acetonitrile and 0.1% TFA in water (⁶⁸Ga-ProBOMB1); 35% acetonitrile and 0.1% TFA in water (⁶⁸Ga-NeoBOMB1); flow rate: 2 mL/min. Retention times: 7.9 min (⁶⁸Ga-ProBOMB1); 9.4 min (⁶⁸Ga-NeoBOMB1).

1.1.1.2 ¹⁷⁷Lu-ProBOMB1.

473.6-932.4 MBq of [¹⁷Lu]LuCl₃ was added to 25 μg of ProBOMB1 in 0.5 ml of sodium acetate buffer (0.1 M; pH 4.5) and incubated at 95° C. for 15 minutes.

Thereafter, the mixture was injected into HPLC to separate the radioligand from unreacted [¹⁷Lu]LuCl₃ and unlabeled precursor (semi-preparative column; 22% acetonitrile and 0.1% TFA in water; flow rate 4.5 mL/min, retention time: 23.9 min). Determination of molar activity was conducted using the analytical column (24% acetonitrile and 0.1% TFA in water; flow rate: 2.0 mL/min, retention time: 7.6 min).

1.1.2 Synthesis of Fmoc-p-aminomethylaniline.

FmocOSu (10.12 g, 30 mmol) in 60 mL acetonitrile was added dropwise to a solution of 4-aminobenzylamine (3.67 g, 30 mmol) and triethylamine (2.79 mL, 30 mmol) in 30 mL acetonitrile and stirred overnight. Water (100 mL) was added to the reaction mixture and the precipitate was collected after filtration. The precipitate was washed thrice with ethanol/ether (1:1, 50 mL) and dried under vacuum to obtain the product as white powder (yield: 5.5 g, 53%). ¹H NMR (300 MHz, DMSO-d₆) δ 7.89 (d, J=7.4 Hz, 2H, Ar), 7.70 (d, J=7.4 Hz, 2H, Ar), 7.42 (t, J=7.4 Hz, 2H, Ar), 7.32 (t, J=7.5 Hz, 2H, Ar), 6.89 (d, J=8.2 Hz, 2H, Ar), 6.50 (d, J=8.2 Hz, 2H, Ar), 4.94 (s, 2H, NH₂), 4.31 (d, J=6.9 Hz, 2H, OCH₂), 4.21 (t, J=6.8 Hz, 1H, CH₂CH), 4.00 (d, J=6.0 Hz, 2H, NHCH₂). ESI-MS: calculated [M+H]⁺ for Fmoc-p-aminomethylaniline C₂₂H₂₀N₂O₂ 345.2; found 345.2.

1.1.3 Synthesis of Fmoc-p-aminomethylaniline Diglycolate.

Diglycolic anhydride (1.09 g, 9.4 mmol) was added to a suspension of Fmoc-p-aminomethylaniline (2.94 g, 8.6 mmol) in dichloromethane (30 mL). The reaction mixture was stirred for 2 hours and filtered. The collected solid was washed thrice with dichloromethane (50 mL) and dried under vacuum to obtain the product as white powder (yield: 2.87 g, 73%). ¹H NMR (300 MHz, DMSO-d₆) b 9.87 (s, 1H, NH), 7.89 (d, J=7.4 Hz, 2H, Ar), 7.80 (t, J=6.0 Hz, 1H, NHCH₂), 7.69 (d, J=7.4 Hz, 2H, Ar), 7.57 (d, J=8.4 Hz, 2H, Ar), 7.42 (t, J=7.3 Hz, 2H, Ar), 7.32 (t, J=7.3 Hz, 2H, Ar), 7.15 (d, J=8.4 Hz, 2H, Ar), 4.35 (d, J=6.8 Hz, 2H, OCH₂), 4.27-4.22 (m, 1H, CH₂CH), 4.22-4.19 (m, 2H, NHCH₂), 4.18-4.08 (m, 4H, O(CH₂)₂). ESI-MS: calculated [M+H]⁺ for Fmoc-p-aminomethylaniline diglycolate C₂₆H2₄N₂O₆ 461.2; found 461.3.

1.1.4 Synthesis of ProBOMB1.

ProBOMB1 was synthesized on solid-phase using Fmoc-based approach. Rink amide-MBHA resin (0.3 mmol) was treated with 20% piperidine in N,N-dimethylformamide (DMF) to remove Fmoc protecting group. Fmoc-Pro-OH pre-activated with HATU (3 eq), HOAt (3 eq), and N,N-diisopropylethylamine (DIEA, 6 eq) was coupled to the resin. After removal of Fmoc protecting group, Fmoc-Leu-aldehyde synthesized per published procedures (10 eq), was coupled to the resin by reductive amination in the presence of excess sodium cyanoborohydride (33 eq) in 5 mL DMF (1% acetic acid). Fmoc-His(Trt)-OH, Fmoc-Gly-OH, Fmoc-Val-OH, Fmoc-Ala-OH, Fmoc-Trp(Boc)-OH, Fmoc-Gln(Trt)-OH, Fmoc-D-Phe-OH (pre-activated with HATU (3 eq), HOAt (3 eq) and DIEA (6 eq)), Fmoc-protected pABzA-DIG linker (pre-activated with HATU (3 eq) and DIEA (6 eq)), and DOTA (pre-activated with HATU (3 eq) and DIEA (6 eq)) were coupled to the resin sequentially. The peptide was deprotected and cleaved from the resin with a mixture of trifluoroacetic acid (TFA) 81.5%, triisopropylsilane (TIS) 1%, water 5%, 1,2-ethanedithiol (EDT) 2.5%, thioanisole 5%, and phenol 5% for 4 h at room temperature. After filtration, the peptide was precipitated by addition of cold diethyl ether, collected by centrifugation, and purified by HPLC (semi-preparative column; 23% acetonitrile and 0.1% TFA in water, flow rate: 4.5 mL/min). The isolated yield was 1.1%. Retention time: 11.0 min. ESI-MS: calculated [M+H]⁺ for ProBOMB1 C₇₉H₁₁₃N₂₀O₁₉ 1645.8; found 1645.8.

1.1.5 Synthesis of NeoBOMB1.

NeoBOMB1 was synthesized on solid-phase using Fmoc-based approach. BAL resin (1% DVB, 0.3 mmol) was swelled in DMF, drained, and activated by shaking for 10 min in 4 ml of 47.5:47.5:5 methanol/DMF/acetic acid solution. 2,6-Dimethylheptane-4-amine (10 eq) in 2 ml of 1:1 methanol/DMF solution was added and the mixture was shaken for 1 h. Sodium cyanoborohydride (10 eq) was added and the mixture was shaken for 16 h. The reaction vial was drained and washed with dichloromethane and DMF. Fmoc-His(Trt)-OH (3 eq) pre-activated with HATU (3 eq), HOAt (3 eq) and DIEA (8 eq) in DMF (6 mL) was then added to the reaction vial, and shaken for at least 1 h. Fmoc-deprotection was performed using 20% piperidine in DMF. Using a similar procedure, Fmoc-Gly-OH (HATU and HOAt substituted by HBTU and HOBt), Fmoc-Val-OH, Fmoc-Ala-OH, Fmoc-Trp(Boc)-OH, Fmoc-Gln(Trt)-OH, Fmoc-D-Phe-OH, Fmoc-protected pABzA-DIG linker, and DOTA were subsequently coupled to the peptide sequence. The peptide was cleaved with a mixture of 82.5/5/2.5/5/5 TFA/water/EDT/thioanisole/phenol and purified by HPLC (Agilent 1260 Infinity II Preparative System) using the preparative column (Gemini® 5 μm NX-C18 110 Å, LC Column 50×30 mm; 29-30.5% acetonitrile and 0.1% TFA in water in 10 minutes and held at 30.5% acetonitrile and 0.1% TFA afterwards; flow rate: 30 mL/min). The isolated yield was 39%. Retention time: 9.0 min. ESI-MS: calculated [M+H]⁺ for NeoBOMB1 C₇₇H₁₁₁N₁₈O₁₈ 1575.8; found 1576.0.

1.1.6 Synthesis of Non-Radioactive Standards.

ProBOMB1 (1.3 mg, 0.79 μmol) and GaCl₃ (0.284 M, 13.9 μL, 3.90 μmol) in 500 μL sodium acetate buffer (0.1 M, pH 4.2) was incubated at 80° C. for 15 min, and purified by HPLC using the semi-preparative column (23% acetonitrile and 0.1% TFA in water; flow rate: 4.5 mL/min). The isolated yield was 67%. Retention time: 15.7 min. ESI-MS: calculated [M+H]⁺ for Ga-ProBOMB1 C₇₉H₁₁₀N₂₀O₁₉Ga 1711.8; found 1711.7. NeoBOMB1 (2.0 mg, 1.17 μmol) and GaCl₃ (0.265 M, 47 μL, 12.46 μmol) in 460 μL sodium acetate buffer (0.1 M, pH 4.2) and 60 μL acetonitrile, was incubated at 80° C. for 15 min, and purified by HPLC using the preparative column (30% acetonitrile and 0.1% TFA in water; flow rate: 30 mL/min. The isolated yield was 38%. Retention time: 13.0 min. ESI-MS: calculated [M+H]⁺ for Ga-NeoBOMB1 C₇₇H₁₀₉N₁₃O₁₃Ga 1643.7; found 1644.0.

1.1.7 Log D_{7.4} Measurements.

Log D_{7.4} values of radiolabeled peptides were measured using the shake flask method as previously reported (24).

1.1.8 Cell Culture.

The PC-3 prostate adenocarcinoma cell line (ATCC-CRL-1435) was cultured in a humidified incubator (5% CO₂; 37° C.) in F-12K medium (Life Technologies Corporations) supplemented with 20% fetal bovine serum (Sigma-Aldrich), 100 I.U./mL penicillin, and 100 μg/mL streptomycin (Life Technologies).

1.1.9 Competition Binding Assay.

The in vitro competition binding assay was modified from previously published procedures (25). PC-3 cells were seeded at 2×10⁵ cells/well in 24 well Poly-D-lysine plates 18-24 h prior to the experiment. The growth medium was replaced by 400 μL of reaction medium. Cells were incubated 30-60 min at 37° C. Non-radioactive peptides in 50 μL of decreasing concentrations (10 μM to 1 μM) and 50 μL 0.011 nM [¹²⁵I-Tyr⁴]Bombesin were added to wells. The cells were incubated with moderate agitation for 1 h at 27° C., washed thrice with ice-cold PBS, harvested by trypsinization, and measured for activity on the gamma counter. Data were analyzed using non-linear regression (one binding site model for competition assay) with GraphPad Prism 7.

1.1.10 Fluorometric Calcium Release Assay.

Calcium release assays were performed using a FLIPR Calcium 6 assay kit (Molecular Devices) according to published procedures (26). Briefly, 5×10⁴ PC-3 cells were seeded overnight in 96-well clear bottom black plates. The growth medium was replaced with loading buffer containing a calcium-sensitive dye and incubated for 30 min at 37° C. The plate was placed in a FlexStation 3 microplate reader (Molecular Devices) and baseline fluorescent signals were acquired for 15 sec. 5 or 50 nM of Ga-ProBOMB1, [D-Phe⁶, Leu-NHEt¹³, des-Met¹⁴]Bombesin(6-14), Bombesin, adenosine triphosphate (ATP, positive control) or PBS (negative control) was added to the cells, and the fluorescent signals were acquired for 105 sec. The relative fluorescence unit (RFU=Max-Min) was used to determine the agonistic/antagonistic properties.

1.1.11 Animal Model.

Animal experiments were approved by the Animal Ethics Committee of the University of British Columbia. Male NOD.Cg-Rag1^{tm1Mom}∥2rg^{tm1Wjl}/SzJ (NRG) mice obtained from an in-house colony were subcutaneously inoculated with 5×10⁶ PC-3 cells (100 μL; 1:1 PBS/Matrigel), and tumors were grown for 2 to 3 weeks.

1.1.12 PET/CT Imaging.

PC-3 tumor-bearing mice were sedated (2.5% isoflurane in O₂) for i.v. injection of radiotracer (4.67±0.91 MBq) with or without 100 μg [D-Phe⁶, Leu-NHEt¹³, des-Met¹⁴]Bombesin(6-14). Mice were sedated and scanned (Siemens Inveon microPET/CT) with body temperature maintained by heating pad. The CT scan was obtained (80 kV; 500 μA; 3 bed positions; 34% overlap; 220° continuous rotation) followed by a 10 min static PET at 1 or 2 h post-injection (p.i.) of the radiotracer. PET data were acquired in list mode, reconstructed using 3-dimensional ordered-subsets expectation maximization (2 iterations) followed by a fast maximum a priori algorithm (18 iterations) with CT-based attenuation correction. Images were analyzed using the Inveon Research Workplace software (Siemens Healthineers).

1.1.13 Biodistribution.

PC-3 tumor-bearing mice were anesthetized (2.5% isoflurane in 02) for i.v. injection of radiotracer (1.84±0.99 MBq) with or without 100 μg of [D-Phe⁶, Leu-NHEt¹³, des-Met¹⁴]Bombesin(6-14). The mice were sacrificed by CO₂ inhalation at 30 min, 1 h, and 2 h p.i. Blood was collected by cardiac puncture. Organs/tissues were harvested, rinsed with PBS, blotted dry, and weighed. The activity in tissues was assayed by gamma counter and expressed as the percentage injected dose per gram of tissue (% ID/g).

1.1.14 In Vivo Stability.

⁶⁸Ga-ProBOMB1 (16.1±2.9 MBq) was intravenously injected into two male NRG mice. After a 5-min uptake period, mice were sedated/euthanized, and blood was collected. The plasma was isolated and analyzed with radio-HPLC (24% acetonitrile and 0.1% TFA in water; flow rate: 2.0 mL/min) following published procedures (26). Retention time of ⁶⁸Ga-ProBOMB1: 8.8 min.

1.1.15 Dosimetry.

Biodistribution data (% ID/g) were decayed to the appropriate time-point and fitted to monoexponential or biexponential models using a Python script developed in-house (Python Software Foundation, v3.5). The choice of fit was based on R² and residuals. The resulting time-activity curve was integrated to obtain the residence time which, multiplied by the model organ mass (25 g MOBY mouse phantom), provided OLINDA (Hermes Medical Solution, v2.0) with input values to calculate dosimetry (27,28).

1.1.16 Statistical Analysis.

The binding affinity was analyzed with one-way ANOVA with a post-hoc t-test on GraphPad Prism 7. Statistics for biodistribution data were computed using R (R Foundation for Statistical Computing, v.3.4.2). Outliers were identified with one round of Grubbs' test (threshold: p<0.01). The Shapiro-Wilk test was used to determine if distributions were normal (threshold: p>0.05); if they were, Welch's t-test was used, or Wilcoxon's test otherwise. Multiple comparisons were corrected by Holm's method.

1.2 Results

1.2.1 Chemistry, Radiolabeling, and Hydrophilicity.

The radiolabeling precursors ProBOMB1 and NeoBOMB1 were obtained in 1.1% and 39% yields, respectively. The non-radioactive standards Ga-ProBOMB1 and Ga-NeoBOMB1 were obtained in 67% and 38% yields, respectively. ⁶⁸Ga-ProBOMB1 was obtained in 48.2±10.9% decayed-corrected isolated yield with 121±46.9 GBq/μmol molar activity and 96.9±1.4% radiochemical purity (n=6). ⁶⁸Ga-NeoBOMB1 was obtained in 34.0±11.8% decayed-corrected isolated yield with 239±87.3 GBq/μmol molar activity and 96.4±0.8% radiochemical purity (n=3). Log D_{7.4} values of ⁶⁸Ga-ProBOMB1 and ⁶⁸Ga-NeoBOMB1 were −2.34±0.05 and −0.88±0.02 (n=3), respectively.

1.2.2 Binding Affinity and Agonist/Antagonist Characterization

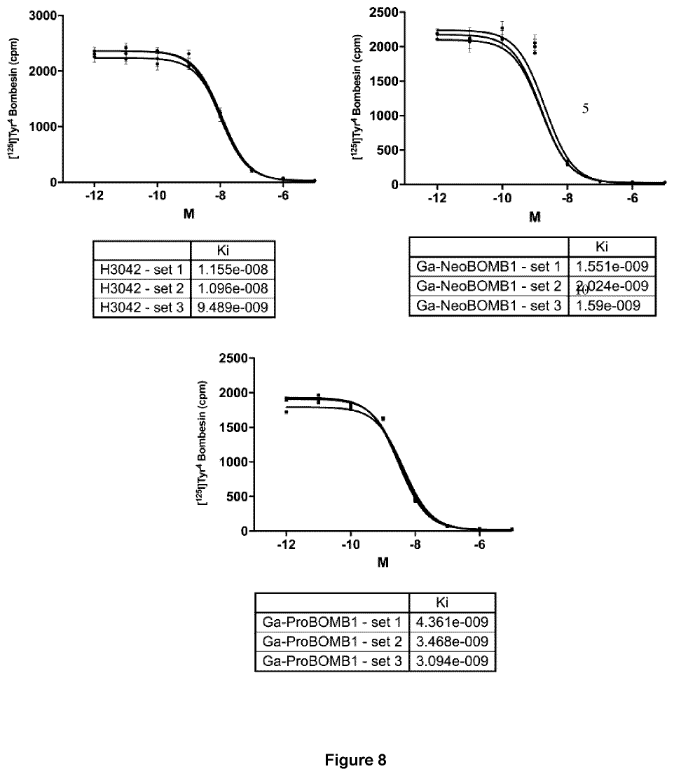

The binding affinities of [D-Phe⁶, Leu-NHEt¹³, des-Met¹⁴]Bombesin(6-14), Ga-ProBOMB1, and Ga-NeoBOMB1 for GRPR were measured in PC-3 cells (FIG. 8). The compounds successfully displaced binding of [¹²⁵I-Tyr⁴]Bombesin in a dose-dependent manner. Ki values for [D-Phe⁶, Leu-NHEt¹³, des-Met¹⁴]Bombesin(6-14), Ga-ProBOMB1, and Ga-NeoBOMB1 were 10.7±1.06, 3.97±0.76, and 1.71±0.28 nM, respectively. Differences in binding affinity were statistically significant between compounds (p<0.05).

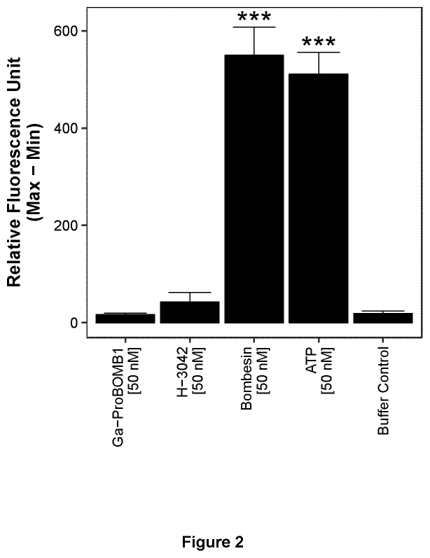

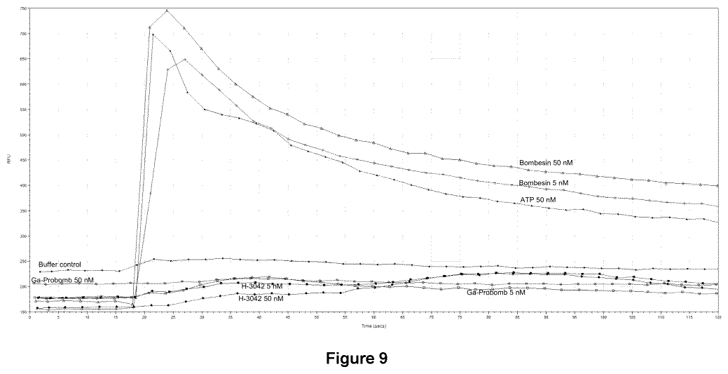

Intracellular calcium release of PC-3 cells was measured for Ga-ProBOMB1 (FIG. 2 and FIG. 9). Bombesin (5 and 50 nM) and ATP (50 nM) induced calcium release corresponding to 535±52.0, 549±58.7, 511±45.5 RFUs, compared to 18.3±5.4 RFUs for buffer control. Differences were statistically significant (p<0.001). For [D-Phe⁶, Leu-NHEt¹³, des-Met¹⁴]Bombesin(6-14) (5 and 50 nM), 22.3±16.8 and 42.0±20.4 RFUs were observed, while 22.3±14.4 and 16.0±3.7 RFUs were observed for Ga-ProBOMB1 (5 and 50 nM). Differences compared to buffer control were not statistically significant.

1.2.3 PET Imaging

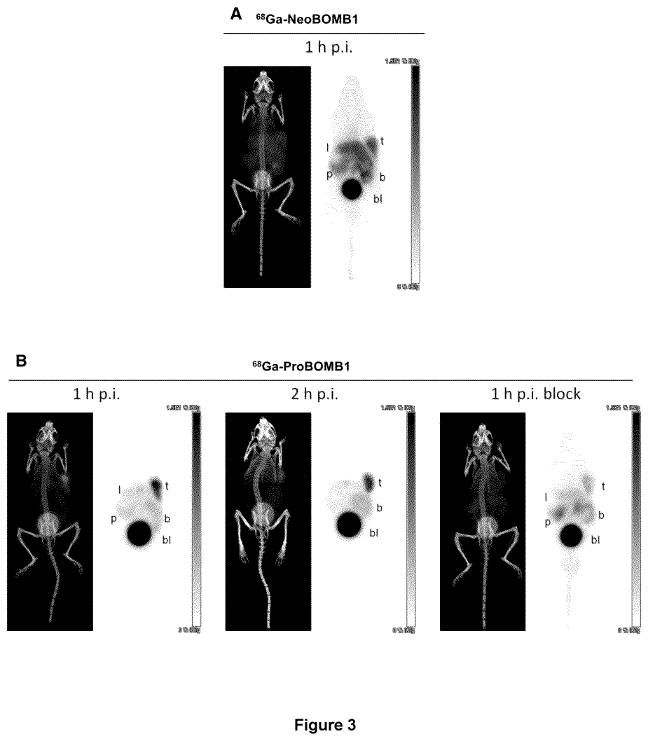

Biodistribution, and Stability. Representative maximum intensity projection PET/CT images (1 and 2 h p.i.) are shown in FIG. 3. ⁶⁸Ga-ProBOMB1 and ⁶⁸Ga-NeoBOMB1 enabled clear visualization of PC-3 tumor xenografts. ⁶⁸Ga-NeoBOMB1 was excreted via both the hepatobiliary and renal pathways, while ⁶⁸Ga-ProBOMB1 was primarily cleared through the renal pathway. For ⁶⁸Ga-ProBOMB1, the highest activity was observed in bladder followed by tumor. For ⁶⁸Ga-NeoBOMB1, activity was observed in tumor, liver, pancreas, bowel, and bladder. Faster clearance of ⁶⁸Ga-ProBOMB1 compared to ⁶⁸Ga-NeoBOMB1 led to higher contrast images. Co-injection of [D-Phe⁶, Leu-NHEt¹³, des-Met¹⁴]Bombesin(6-14) decreased average uptake of ⁶⁸Ga-ProBOMB1 in tumors by 62%.

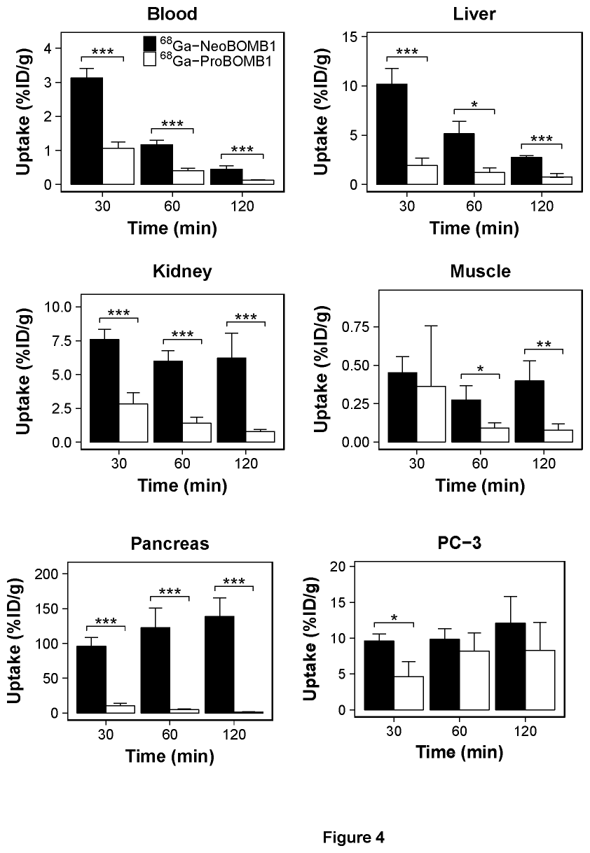

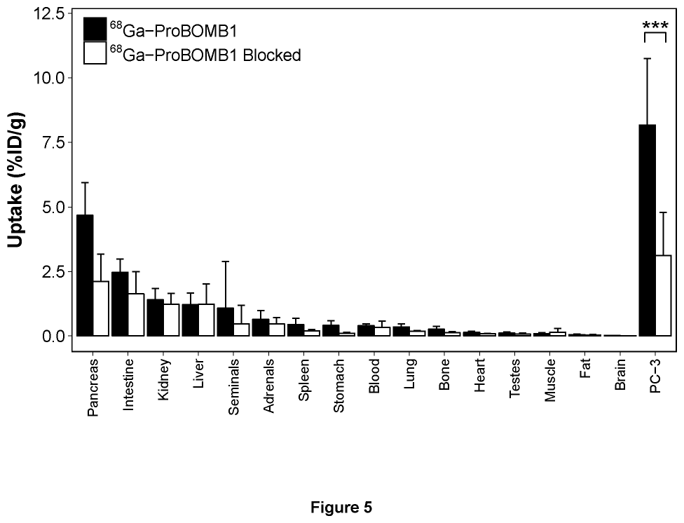

For biodistribution, uptake (% ID/g) of selected organs for ⁶⁸Ga-NeoBOMB1 and ⁶⁸Ga-ProBOMB1 were compared (FIG. 4). Thirty minutes p.i., PC-3 tumor uptake was lower for ⁶⁸Ga-ProBOMB1 (4.62±2.13) than for ⁶⁸Ga-NeoBOMB1 (9.60±0.99) (p<0.001). Tumor uptake of ⁶⁸Ga-ProBOMB1 was 8.17±2.13 at 60 min and 8.31±3.88 at 120 min, and that of ⁶⁸Ga-NeoBOMB1 was 9.83±1.48 at 60 min and 12.1±3.72 at 120 min (not significantly different). Uptake of blood, liver, pancreas, and kidney for ⁶⁸Ga-ProBOMB1 was lower than for ⁶⁸Ga-NeoBOMB1 at all time-points (p<0.05). In particular, pancreatic uptake was markedly lower at 30, 60, and 120 min for ⁶⁸Ga-ProBOMB1 (respectively: 10.4±3.79, 4.68±1.26, 1.55±0.49) compared with ⁶⁸Ga-NeoBOMB1 (respectively: 95.7±12.7, 122±28.4, 139±26.8). Muscle uptake was only significantly lower in ⁶⁸Ga-ProBOMB1 vs ⁶⁸Ga-NeoBOMB1 at 60 and 120 min (p<0.01). For all other collected organs (Table 6 and 7), with the exception of seminal vesicles at 60 min, there was less uptake with ⁶⁸Ga-ProBOMB1 than ⁶⁸Ga-NeoBOMB1, although that was not always statistically significant. When co-injected with [D-Phe⁶, Leu-NHEt¹³, des-Met¹⁴]Bombesin(6-14) (FIG. 5), tumor uptake of ⁶⁸Ga-ProBOMB1 at 60 min was significantly reduced to 3.12±1.68 (p<0.01). Injected radiolabeled peptide mass for ⁶⁸Ga-NeoBOMB1 (6.01±2.89 μmol) and ⁶⁸Ga-ProBOMB1 (20.24±12.9 μmol) was different (p<0.001), but had overlapping ranges at 30 and 60 min.

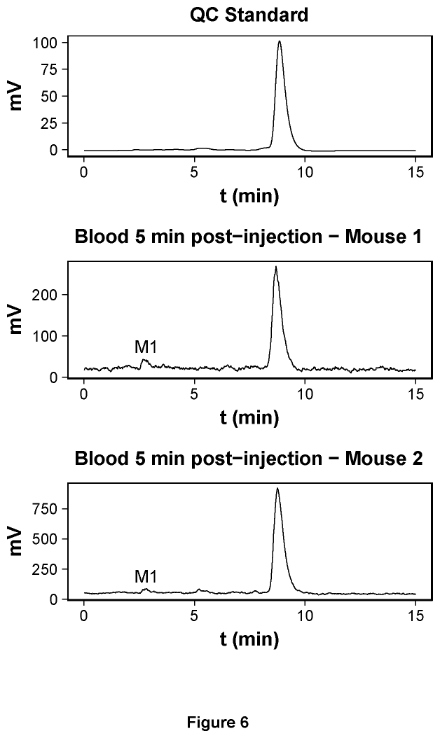

The stability of ⁶⁸Ga-ProBOMB1 was measured in plasma at 5 min post-injection. According to HPLC results (FIG. 6), ⁶⁸Ga-ProBOMB1 (t_{R}=8.84 min) was 96.3±2.7% intact. A minor metabolite peak was observed at t_{R}=2.72 min.

1.2.4 Dosimetry

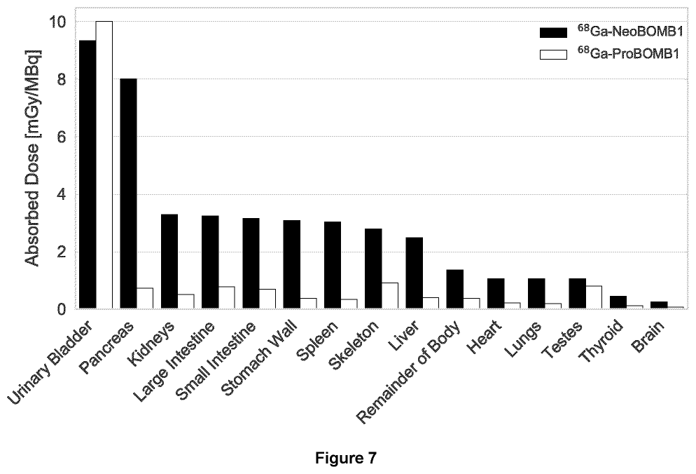

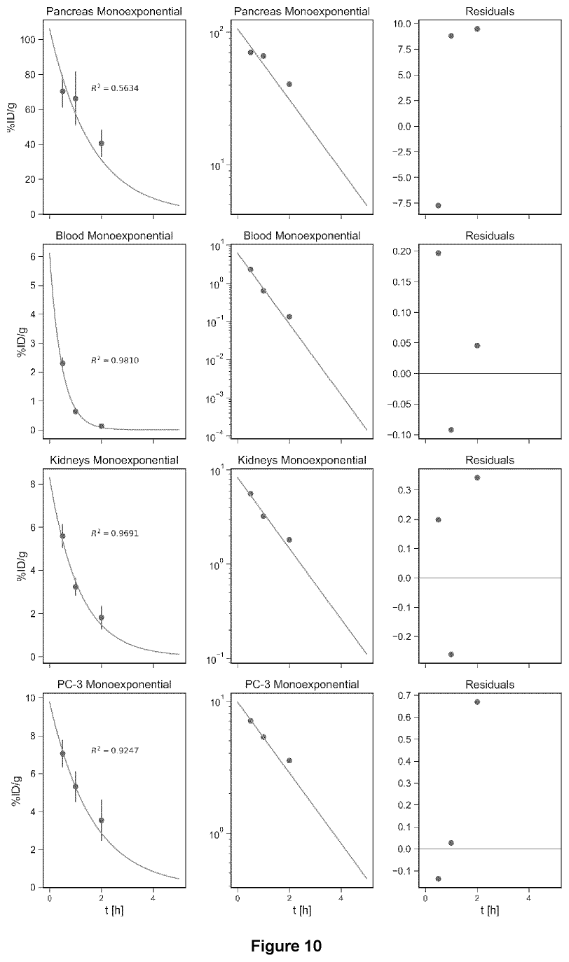

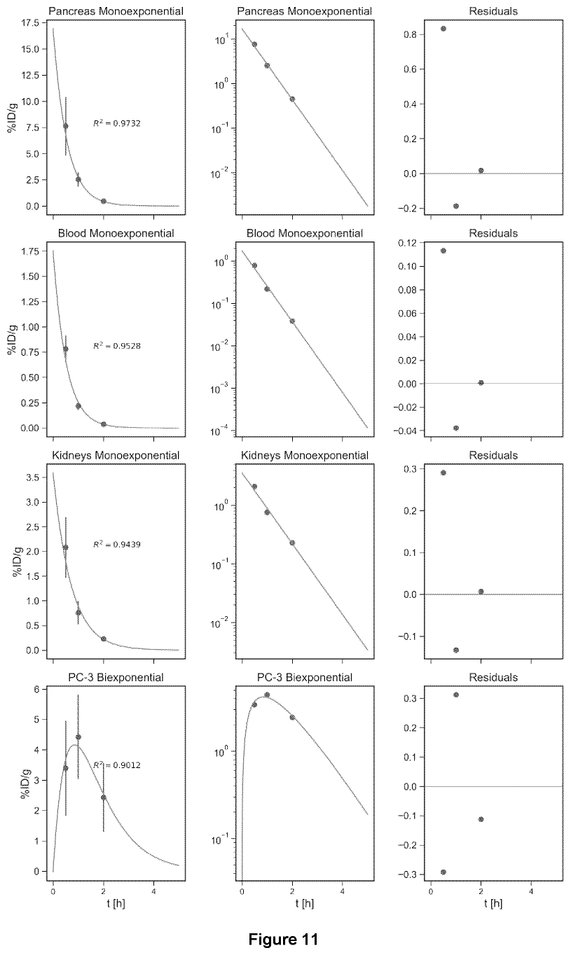

The absorbed doses in mice are shown in FIG. 7 and Table 8, based on kinetic curves derived from biodistribution data (FIGS. 10 and 11). The organ that received the highest dose from ⁶⁸Ga-ProBOMB1 was the urinary bladder (10.00 mGy/MBq). Besides the urinary bladder, all other organs received less than 1 mGy/MBq. Higher doses were observed for ⁶⁸Ga-NeoBOMB1 in most organs including pancreas (8.00 mGy/MBq), kidneys (3.29 mGy/MBq), large and small intestines (3.24 and 3.15 mGy/MBq).

The estimated absorbed whole-body dose for an average adult human male was also computed (Table 5). Consistent with the mouse model, higher doses were obtained for ⁶⁸Ga-NeoBOMB1 than ⁶⁸Ga-ProBOMB1 across all organs except bladder (5.69×10⁻² vs. 6.59×10⁻² mGy/MBq). Notably, the pancreas is expected to receive 2.63×10⁻¹ mGy/MBq for ⁶⁸Ga-NeoBOMB1 vs 1.44×10⁻² mGy/MBq for ⁶⁸Ga-ProBOMB1. The kidney is expected to receive 1.69×10⁻² mGy/MBq for ⁶⁸Ga-NeoBOMB1 vs 4.32×10−3 mGy/MBq for ⁶⁸Ga-ProBOMB1.

ProBOMB1 and the non-radioactive Ga-ProBOMB1 were obtained in 1.1 and 67% yield, respectively. The Ki value of Ga-ProBOMB1 for GRPR was 3.97±0.76 nM. Ga-ProBOMB1 retained antagonist properties after modifications. ⁶⁸Ga-ProBOMB1 was obtained in 48.2±10.9% decay-corrected radiochemical yield with 121±46.9 GBq/μmol molar activity, and >95% radiochemical purity. Imaging/biodistribution studies showed excretion of ⁶⁸Ga-ProBOMB1 was primarily through the renal pathway. At 1 h post-injection (p.i.), PC-3 tumor xenografts were clearly delineated in PET images with excellent contrast. Based on biodistribution data at 1 h p.i., tumor uptake for ⁶⁸Ga-ProBOMB1 was 8.17±2.57 percent injected dose per gram (% ID/g), and 9.83±1.48% ID/g for ⁶⁸Ga-NeoBOMB1. This corresponded to tumor-to-blood and tumor-to-muscle uptake ratios of 20.6±6.79 and 106±57.7 for ⁶⁸Ga-ProBOMB1, and 8.38±0.78 and 39.0±12.6 for ⁶⁸Ga-NeoBOMB1. Blockade with [D-Phe⁶, Leu-NHEt¹³, des-Met¹⁴]Bombesin(6-14) significantly reduced average uptake of ⁶⁸Ga-ProBOMB1 in tumors by 62%. The total absorbed dose was lower for ⁶⁸Ga-ProBOMB1 in all organs except bladder compared to ⁶⁸Ga-NeoBOMB1.

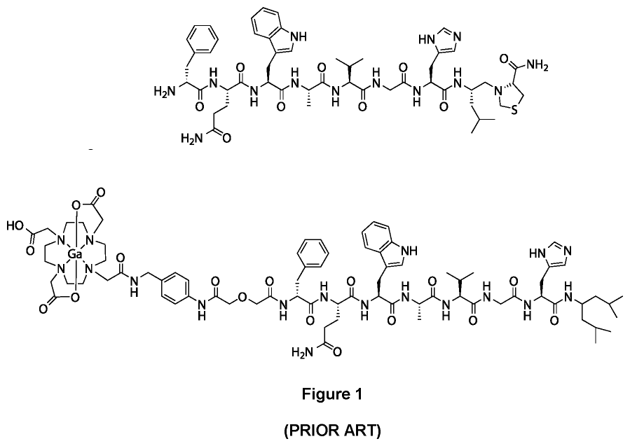

We report the synthesis and biological evaluation of a novel BBN antagonist, ⁶⁸Ga-ProBOMB1 (⁶⁸Ga-DOTA-pABzA-DIG-D-Phe-Gln-Trp-Ala-Val-Gly-His-Leu-L-Pro-NH₂), based on the sequence of the previously reported RC-3950-II (D-Phe-Gln-Trp-Ala-Val-Gly-His-Leu-L-Tac-NH₂; Tac: 4-thiazolidinecarboxylic acid; FIG. 1).

TABLE 5
Estimated absorbed doses for different organs in the
adult human male calculated with OLINDA software.

| | ⁶⁸Ga-NeoBOMB1 | ⁶⁸Ga-ProBOMB1 |
|---|---|---|
| | Absorbed Dose | Absorbed Dose |
| Target Organ | [mGy/MBq] | [mGy/MBq] |
| Adrenals | 0.041600 | 0.002400 |
| Brain | 0.000316 | 0.000088 |
| Esophagus | 0.002710 | 0.000450 |
| Eyes | 0.000622 | 0.000193 |
| Gallbladder wall | 0.004800 | 0.000959 |
| Left colon | 0.032100 | 0.006150 |
| Small intestine | 0.029300 | 0.005990 |
| Stomach wall | 0.009990 | 0.001030 |
| Right colon | 0.016700 | 0.003440 |
| Rectum | 0.015000 | 0.004080 |
| Heart | 0.006160 | 0.001520 |
| Kidneys | 0.016900 | 0.004320 |
| Liver | 0.018800 | 0.003650 |
| Lungs | 0.013800 | 0.001090 |
| Pancreas | 0.263000 | 0.014400 |
| Prostate | 0.002830 | 0.002100 |
| Salivary glands | 0.000722 | 0.000214 |
| Red marrow | 0.002140 | 0.000685 |
| Skeleton | 0.001440 | 0.000452 |
| Spleen | 0.009440 | 0.001260 |
| Testes | 0.001310 | 0.000880 |
| Thymus | 0.001720 | 0.000356 |
| Thyroid | 0.001220 | 0.000264 |
| Urinary bladder wall | 0.056900 | 0.065900 |
| Remainder of body | 0.003050 | 0.000962 |

TABLE 6
Biodistribution and tumor-to-organ contrasts of ⁶⁸Ga-NeoBOMB1
in PC-3 xenograft bearing mice at selected time-points.

| | 30 min | | | 60 min | | | 120 min | | |
|---|---|---|---|---|---|---|---|---|---|
| | Mean | SD | n | Mean | SD | n | Mean | SD | n |
| Tissues | | | | | | | | | |
| Blood | 3.13 | 0.28 | 5 | 1.17 | 0.12 | 5 | 0.45 | 0.10 | 8 |
| Fat | 0.20 | 0.03 | 5 | 0.17 | 0.04 | 5 | 0.17 | 0.02 | 8 |
| Seminal glands | 0.19 | 0.05 | 4 | 0.56 | 0.71 | 4 | 0.25 | 0.12 | 7 |
| Testes | 0.50 | 0.08 | 5 | 0.29 | 0.07 | 4 | 0.19 | 0.04 | 8 |
| Intestine | 10.61 | 0.86 | 5 | 11.26 | 2.52 | 5 | 10.99 | 1.66 | 8 |
| Stomach | 2.62 | 1.27 | 5 | 2.82 | 1.51 | 5 | 3.46 | 1.49 | 8 |
| Spleen | 3.64 | 3.96 | 5 | 3.35 | 2.89 | 5 | 2.52 | 3.30 | 7 |
| Liver | 10.16 | 1.61 | 5 | 5.14 | 1.28 | 5 | 2.76 | 0.17 | 7 |
| Pancreas | 95.73 | 12.67 | 5 | 122.54 | 28.40 | 5 | 138.70 | 26.75 | 8 |
| Adrenals | 15.24 | 12.35 | 5 | 16.11 | 8.28 | 5 | 19.03 | 7.78 | 8 |
| Kidney | 7.59 | 0.76 | 5 | 5.98 | 0.78 | 5 | 6.21 | 1.85 | 8 |
| Lung | 6.29 | 2.32 | 5 | 7.93 | 4.30 | 5 | 3.31 | 5.07 | 8 |
| Heart | 0.69 | 0.12 | 5 | 0.47 | 0.05 | 5 | 0.34 | 0.02 | 8 |
| Muscle | 0.45 | 0.11 | 5 | 0.27 | 0.09 | 5 | 0.40 | 0.13 | 7 |
| Bone | 0.67 | 0.23 | 5 | 1.01 | 0.43 | 5 | 1.13 | 0.21 | 8 |
| Brain | 0.07 | 0.01 | 5 | 0.07 | 0.03 | 5 | 0.08 | 0.03 | 8 |
| PC-3 Tumor | 9.60 | 0.99 | 5 | 9.83 | 1.48 | 5 | 12.07 | 3.72 | 8 |
| Ratios | | | | | | | | | |
| Tumor-to-Blood | 3.08 | 0.35 | 5 | 8.38 | 0.78 | 5 | 28.42 | 11.30 | 8 |
| Tumor-to-Muscle | 22.33 | 6.04 | 5 | 39.00 | 12.55 | 5 | 30.54 | 8.38 | 7 |
| Tumor-to-Kidney | 1.27 | 0.15 | 5 | 1.66 | 0.26 | 5 | 1.99 | 0.52 | 8 |
| Tumor-to-Pancreas | 0.10 | 0.01 | 5 | 0.08 | 0.03 | 5 | 0.09 | 0.02 | 8 |
| Tumor-to-Liver | 0.96 | 0.20 | 5 | 2.00 | 0.55 | 5 | 4.33 | 1.57 | 7 |

TABLE 7
Biodistribution and tumor-to-organ contrasts of ⁶⁸Ga-ProBOMB1
in PC-3 xenograft bearing mice at selected time-points.

| | 30 min | | | 60 min | | | 120 min | | | 60 min blocked | | |
|---|---|---|---|---|---|---|---|---|---|---|---|---|
| | Mean | SD | n | Mean | SD | n | Mean | SD | n | Mean | SD | n |
| Tissues | | | | | | | | | | | | |
| Blood | 1.06 | 0.18 | 6 | 0.40 | 0.07 | 8 | 0.13 | 0.01 | 6 | 0.33 | 0.24 | 7 |
| Fat | 0.17 | 0.05 | 6 | 0.06 | 0.02 | 8 | 0.07 | 0.05 | 7 | 0.04 | 0.02 | 7 |
| Seminal glands | 0.17 | 0.05 | 6 | 1.08 | 1.82 | 8 | 0.43 | 0.74 | 6 | 0.47 | 0.72 | 7 |
| Testes | 0.31 | 0.06 | 6 | 0.11 | 0.04 | 8 | 0.09 | 0.03 | 7 | 0.07 | 0.03 | 7 |
| Intestine | 2.63 | 0.77 | 6 | 2.47 | 0.51 | 8 | 1.68 | 0.81 | 7 | 1.63 | 0.86 | 7 |
| Stomach | 0.53 | 0.17 | 6 | 0.41 | 0.18 | 8 | 0.18 | 0.06 | 7 | 0.10 | 0.04 | 7 |
| Spleen | 0.50 | 0.21 | 6 | 0.44 | 0.24 | 8 | 0.31 | 0.15 | 7 | 0.20 | 0.06 | 7 |
| Liver | 1.93 | 0.76 | 6 | 1.21 | 0.45 | 8 | 0.75 | 0.37 | 7 | 1.23 | 0.78 | 7 |
| Pancreas | 10.35 | 3.79 | 6 | 4.68 | 1.26 | 8 | 1.55 | 0.49 | 7 | 2.12 | 1.05 | 7 |
| Adrenals | 0.95 | 0.25 | 5 | 0.65 | 0.34 | 8 | 1.23 | 1.33 | 7 | 0.47 | 0.24 | 7 |
| Kidney | 2.83 | 0.84 | 6 | 1.40 | 0.44 | 8 | 0.78 | 0.16 | 7 | 1.22 | 0.43 | 7 |
| Lung | 0.54 | 0.09 | 6 | 0.35 | 0.12 | 7 | 0.25 | 0.09 | 6 | 0.19 | 0.02 | 6 |
| Heart | 0.27 | 0.04 | 6 | 0.14 | 0.05 | 8 | 0.11 | 0.06 | 7 | 0.08 | 0.01 | 6 |
| Muscle | 0.36 | 0.39 | 6 | 0.09 | 0.03 | 8 | 0.08 | 0.04 | 6 | 0.15 | 0.15 | 7 |
| Bone | 0.22 | 0.18 | 6 | 0.26 | 0.11 | 8 | 0.39 | 0.35 | 7 | 0.13 | 0.04 | 7 |
| Brain | 0.02 | 0.00 | 6 | 0.02 | 0.01 | 8 | 0.03 | 0.02 | 7 | 0.01 | 0.00 | 7 |
| PC-3 Tumor | 4.62 | 2.13 | 6 | 8.17 | 2.57 | 8 | 8.31 | 3.88 | 7 | 3.12 | 1.68 | 7 |
| Ratios | | | | | | | | | | | | |
| Tumor-to-Blood | 4.43 | 2.13 | 6 | 20.63 | 6.79 | 8 | 66.57 | 40.07 | 6 | 11.93 | 7.25 | 7 |
| Tumor-to-Muscle | 26.08 | 25.00 | 6 | 105.66 | 57.74 | 8 | 159.62 | 169.58 | 6 | 36.76 | 29.11 | 7 |
| Tumor-to-Kidney | 1.82 | 1.09 | 6 | 6.25 | 2.33 | 8 | 11.20 | 6.72 | 7 | 2.51 | 0.75 | 7 |
| Tumor-to-Pancreas | 0.47 | 0.24 | 6 | 1.80 | 0.55 | 8 | 5.97 | 3.11 | 7 | 1.56 | 0.52 | 7 |
| Tumor-to-Liver | 2.91 | 2.21 | 6 | 7.33 | 2.97 | 8 | 13.26 | 5.72 | 7 | 2.69 | 0.67 | 7 |

*Mice received a co-injection of 100 μg of [D-Phe⁶,Leu-NHEt¹³,des-Met¹⁴]Bombesin(6-14).

TABLE 8
Absorbed doses for different organs in
mice calculated with OLINDA software.

| | ⁶⁸Ga-NeoBOMB1 | ⁶⁸Ga-ProBOMB1 |
|---|---|---|
| | Absorbed Dose | Absorbed Dose |
| Target Organ | [mGy/MBq] | [mGy/MBq] |
| Brain | 0.258 | 0.0678 |
| Large intestine | 3.240 | 0.7750 |
| Small intestine | 3.150 | 0.6960 |
| Stomach wall | 3.080 | 0.3700 |
| Heart | 1.060 | 0.2090 |
| Kidneys | 3.290 | 0.5150 |
| Liver | 2.480 | 0.3970 |
| Lungs | 1.060 | 0.1940 |
| Pancreas | 8.000 | 0.7250 |
| Skeleton | 2.790 | 0.9170 |
| Spleen | 3.030 | 0.3440 |
| Testes | 1.050 | 0.7950 |
| Thyroid | 0.455 | 0.1170 |
| Urinary bladder | 9.330 | 10.0000 |
| Remainder of body | 1.360 | 0.3720 |

1.3 Discussion

There is longstanding interest in development of radiopharmaceuticals targeting GRPR, due to overexpression of this receptor in cancer. The overexpression is strongly correlated with estrogen receptor positivity in breast cancer (29), and cohort studies have shown GRPR antagonists to be effective in detecting primary and metastatic lesions in patients (12,30). There is extensive literature supporting the use of GRPR radiopharmaceuticals for prostate cancer in patients (6,9,31). Due to tumor heterogeneity, it has been postulated that GRPR radiotheranostics can complement prostate-membrane specific antigen (PSMA) agents to improve prostate cancer management (32,33)

We synthesized ⁶⁸Ga-ProBOMB1 based on the sequence of RC-3950-II, a [Leu¹³ψAA¹⁴]BBN derivative (17). We replaced the last amino acid Tac¹⁴ with Pro¹⁴, as proline was readily available in our lab and shows good structural homology (FIG. 1). Compared to the native BBN sequence, RC-3950-II has a D-Phe⁶ substitution which enhances binding potency (34), and is present in other antagonists like RM2 (15) and NeoBOMB1 (13,35). The radiometal/chelator complex (⁶⁸Ga-DOTA) was appended at the N-terminus of the GRPR-targeting sequence and separated by a pABzA-DIG linker, a modular design that parallels that of ⁶⁸Ga-NeoBOMB1. Recently, Nock et al. presented the first-in man study in four prostate cancer patients (13). ⁶⁸Ga-NeoBOMB1 was well-tolerated and generated high-contrast PET images. The tracer successfully localized to the primary prostate tumor and distant metastatic sites (lymph nodes, liver, and bone). The authors are exploring the use of ¹⁷⁷Lu-labeled NeoBOMB1 for peptide receptor radionuclide therapy.

The Kᵢ value of Ga-ProBOMB1 for GRPR (3.97±0.76 nM) was approximately two-fold higher than Ga-NeoBOMB1. It was also higher than the reported value for RC-3950-II (0.078 nM); however, the latter value was determined using Swiss 3T3 cells (17). We proceeded to study the agonist/antagonist properties of Ga-ProBOMB1 using a calcium efflux assay (FIG. 2). While BBN and ATP significantly induced intracellular calcium release (>500 RFUs) compared to buffer control (18.3±5.4 RFUs), Ga-ProBOMB1 behaved as an antagonist and did not significantly induce calcium release (16.0±3.7 RFUs). For GRPR, this property is important for tolerability in humans. Moreover, for selected peptide-receptor systems like somatostatin, there is a paradigm shift favoring the use of antagonists over agonists for tumor targeting (36).

PET imaging demonstrated that ⁶⁸Ga-ProBOMB1 and ⁶⁸Ga-NeoBOMB1 were able to detect GRPR-expressing PC-3 prostate cancer xenografts (FIG. 3). ⁶⁸Ga-ProBOMB1 cleared rapidly through the renal pathway to yield high-contrast images at 1 h p.i. (post-injection). We noted that tumor uptake was retained at 2 h p.i. for ⁶⁸Ga-ProBOMB1, in conjunction with a further reduction in background activity. This suggests the optimal imaging window can be extended beyond 1 h timepoint without compromising sensitivity or contrast. Target specificity was confirmed with successful tumor blockade with [D-Phe⁶, Leu-NHEt¹³, des-Met¹⁴]Bombesin(6-14).

Our biodistribution data were in agreement with PET imaging studies (FIGS. 4 and 5). The uptake of ⁶⁸Ga-ProBOMB1 (% ID/g) in tumor increased from 4.62±2.13 at 30 min to 8.31±3.88 at 2 h. Similarly, the uptake of ⁶⁸Ga-NeoBOMB1 in tumor increased from 9.60±0.99 at 30 min to 12.1±3.72 at 2 h. ⁶⁸Ga-ProBOMB1 showed slower tumor targeting and accumulation, but faster clearance from blood (0.13±0.01 vs. 0.45±0.10 at 2 h) than ⁶⁸Ga-NeoBOMB1. Subsequently, better contrast ratios were observed for ⁶⁸Ga-ProBOMB1. At 1 h p.i., the contrast ratios for tumor-to-blood, tumor-to-muscle, tumor-to-kidney, and tumor-to-liver were 20.6±6.79 vs 8.38±0.78, 106±57.7 vs 39.0±12.6, 6.25±2.33 vs 1.66±0.26, and 7.33±2.97 vs 0.08±0.03, respectively. The slightly lower uptake of ⁶⁸Ga-ProBOMB1 in tumor xenografts can be explained by its lower binding affinity to GRPR, while the better contrast can be attributed to differences in hydrophilicity. Interestingly, we observed significantly lower pancreas uptake for ⁶⁸Ga-ProBOMB1 (4.68±1.26 and 1.55±0.49% ID/g at 1 and 2 h) compared to ⁶⁸Ga-NeoBOMB1 (123±28.4 and 139±26.8% ID/g at 1 and 2 h). The results obtained for ⁶⁸Ga-NeoBOMB1 were comparable to those presented by Dalm et al. (35), with the exception of the higher pancreas uptake noted in our study. The high pancreas uptake of ⁶⁸Ga-NeoBOMB1 can potentially be attributed to differences in molar activity and/or mouse strain. Dalm et al. injected 250 μmol of ⁶⁸Ga-NeoBOMB1 for biodistribution studies, and uptake in tumor and pancreas were approximately ˜10 and 15% ID/g respectively in nude mice bearing PC-3 tumors (35). From the same paper, when greater mass of peptide was injected for ¹⁷⁷Lu-NeoBOMB1 (200 vs 10 μmol), pancreas uptake was reduced.

A general limitation of BBN-based radiopharmaceuticals is their metabolic stability, as BBN is susceptible to enzymatic cleavage by neutral endopeptidase (37,38). ⁶⁸Ga-ProBOMB1 was >95% stable in plasma at 5 min p.i. While a minor hydrophilic metabolite peak was observed, its identity was not interrogated in this study. The stability of the compound is promising for translation, or for repositioning as a radiotherapeutic agent. The DOTA chelator can form stable complexes with therapeutic trivalent radiometals like ⁹⁰Y or ¹⁷⁷Lu, to create a theranostic pair.

Dosimetry was calculated for mice and extrapolated to the adult human male. When compared with ⁶⁸Ga-NeoBOMB1, the absorbed dose for ⁶⁸Ga-ProBOMB1 in mice was lower across all organs except for urinary bladder (9.33 vs 10.00 mGy/MBq). With ⁶⁸Ga-ProBOMB1, mice received approximately one-sixth and one-tenth the estimated absorbed dose for kidneys and pancreas. For the human model, lower doses were also obtained for ⁶⁸Ga-ProBOMB1. Accordingly, the average adult male is predicted to receive approximately one-quarter and one-twentieth the absorbed dose for kidneys and pancreas, respectively.

1.4 Conclusion

We synthesized a novel GRPR imaging agent, ⁶⁸Ga-ProBOMB1, based on the [Leu¹³ψAA¹⁴]BBN family. The radiopharmaceutical exhibited nanomolar affinity for GRPR and high stability in vivo. ⁶⁸Ga-ProBOMB1 was able to generate high-contrast PET images with good tumor uptake in a prostate cancer model. ⁶⁸Ga-ProBOMB1 had a better dosimetry profile (enhanced contrast and lower whole-body absorbed dose) compared to ⁶⁸Ga-NeoBOMB1.

### Example 2: ProBOMB2

2.1 Materials and Methods

2.1.1 General Overview of Methods and Approach.

ProBOMB2 (DOTA-Pip-D-Phe-Gln-Trp-Ala-Val-Gly-His-Leu-ψ(CH₂N)-Pro-NH₂) was synthesized by solid-phase peptide synthesis. The polyaminocarboxylate chelator 1,4,7,10-tetraazacyclododecane-1,4,7,10-tetraacetic acid (DOTA) was coupled to the N-terminus and separated from the GRPR-targeting sequence by a 4-amino-(1-carboxymethyl) piperidine (Pip) linker. Binding affinity to GRPR was determined using a cell-based competition assay, while agonist/antagonist property was determined with a calcium efflux assay. ProBOMB2 was radiolabeled with ⁶⁸GaCl3. PET imaging and biodistribution studies were performed in male immunocompromised mice bearing PC-3 prostate cancer xenografts. Blocking experiments were performed with co-injection of [D-Phe⁶, Leu-NHEt¹³, des-Met¹⁴]Bombesin (6-14).

2.1.2 General Methods.

All reagents and solvents were purchased from commercial sources and used without further purification except for Fmoc-Leu-ψ(CH₂N)-Tac-OH, which was synthesized by our lab. [D-Phe⁶, Leu-NHEt¹³, des-Met¹⁴]Bombesin(6-14) and Bombesin were purchased from Bachem and Anaspec, respectively. Other peptides were synthesized on an AAPPTec Endeavor 90 peptide synthesizer. High-performance liquid chromatography (HPLC) was performed on an Agilent 1260 infinity system (model 1200 quaternary pump, model 1200 UV absorbance detector set at 220 nm, Bioscan Nal scintillation detector). HPLC columns used were a semi-preparative column (Luna C18, 5μ, 250×10 mm) and an analytical column (Luna, C18, 5μ, 250×4.6 mm) from Phenomenex. Mass analyses were performed using an AB SCIEX 4000 QTRAP mass spectrometer with an ESI ion source. ⁶⁸Ga was eluted from an iThemba Labs generator and purified according to previously published procedures using a DGA resin column from Eichrom Technologies LLC (24). Radioactivity of ⁶⁸Ga-labeled peptides was measured using a Capintec CRC-25R/W dose calibrator, and the radioactivity in tissues collected from biodistribution studies were counted using a Perkin Elmer Wizard2 2480 gamma counter.

2.1.3 Chemistry and Radiolabeling.

The synthesis procedures for radiolabeling precursors and nonradioactive standards are shown below. Purified ⁶⁸GaCl₃ (289-589 MBq, in 0.6 mL water) was added to 0.6 mL of HEPES buffer (2 M, pH 5.3) containing ProBOMB2. The mixture was heated by microwave oven (Danby DMW7700WDB; power setting 2; 1 min). HPLC purification was used to separate ⁶⁸Ga-labeled product from the unlabeled precursor (semi-preparative column.

2.1.4 Synthesis of Fmoc-Leu-ψ(CH₂N)-Tac-OH.

Fmoc-Leucinol (1.1 g g, 3.24 mmol) in 50 ml dichloromethane was stirred with Dess-Martin Periodinane (2.7 g, 6.36 mmol) at room temperature for 4 hours. Reaction mixture was concentrated in vacuo before adding hexanes (70 mL) and saturated sodium bicarbonate solution (30 mL) and stirring for 15 minutes before filtering. Filtrate was washed with saturated sodium bicarbonate solution (3×50 mL), water (3×50 mL), and brine (3×50 mL). Organic layer was collected and dried over magnesium sulfate, filtered, and evaporated in vacuo to obtain crude crystalline compound. The isolated solid was dissolved in 36 mL dichloroethane with L-Proline (410 mg, 3.56 mmol) and the mixture stirred for 48 h at room temperature. Sodium triacetoxyborohydride (1.7 g, 8.1 mmol) was added to the mixture and stirred further for 16 h. The solution was then concentrated in vacuo and ethyl acetate and saturated sodium bicarbonate was added (1:1, 50 mL) and the mixture stirred for 10 min. The organic layer was washed with saturated sodium bicarbonate solution (3×50 mL), water (3×50 mL), and brine (3×50 mL). The organic layer was dried over MgSO₄ before concentrating under vacuum to obtain yellow crude solid. Crude material was purified using HPLC (Phenomenex Gemini Prep column, 38% acetonitrile and 0.1% TFA in water, flow rate 30 mL/min). Retention time: 9.8 min. Product peak was collected and lyophilized to obtain the product as white powder (yield: 436 mg, 31% yield).ESI-MS: calculated [M+H]⁺ for Fmoc-LeuψTac C₂₆H3₂N₂O₄ 437.2; found 437.3.

2.1.5 Synthesis of ProBOMB2.

ProBOMB2 was synthesized on solid-phase using Fmoc-based approach. Rink amide-MBHA resin (0.1 mmol) was treated with 20% piperidine in N,N-dimethylformamide (DMF) to remove Fmoc protecting group. Fmoc-Leu-ψ(CH₂N)-Pro-OH (shown below) pre-activated with HATU (3 eq), HOAt (3 eq), and N,N-diisopropylethylamine (DIEA, 6 eq) was coupled to the resin. After removal of Fmoc protecting group, Fmoc-His(Trt)-OH, Fmoc-Gly-OH, Fmoc-Val-OH, Fmoc-Ala-OH, Fmoc-Trp(Boc)-OH, Fmoc-Gln(Trt)-OH, Fmoc-D-Phe-OH (pre-activated with HATU (3 eq), HOAt (3 eq) and DIEA (6 eq)), Fmoc-protected Pip linker (pre-activated with HATU (3 eq) and DIEA (6 eq)), and DOTA (pre-activated with HATU (3 eq) and DIEA (6 eq)) were coupled to the resin sequentially. The peptide was deprotected and cleaved from the resin with a mixture of trifluoroacetic acid (TFA) 92.5%, triisopropylsilane (TIS) 2.5%, water 2.5%, 2,2′-(ethylenedioxy)diethanethiol (DODT) 2.5% for 4 h at room temperature. After filtration, the peptide was precipitated by addition of cold diethyl ether, collected by centrifugation, and purified by HPLC (semi-preparative column; 20% acetonitrile and 0.1% TFA in water, flow rate: 4.5 mL/min). The isolated yield was 2.4%. Retention time: 16.8 min. ESI-MS: calculated [M+2H]⁺ for C₇₅H₁₁₂N₂₀O₁₇Ga ProBOMB2: 1567.8; found 1567.4.

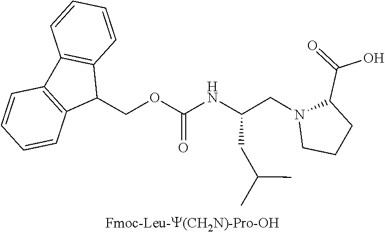

2.1.6 Synthesis of Non-radioactive Standards.

ProBOMB2 (1.8 mg, 1.15 μmol) and GaCl₃ (0.2 M, 28.5 μL, 5.75 μmol) in 450 μL sodium acetate buffer (0.1 M, pH 4.2) was incubated at 80° C. for 30 min, and purified by HPLC using the semi-preparative column (20% acetonitrile and 0.1% TFA in water; flow rate: 4.5 mL/min). The isolated yield was 88%. Retention time: 12.1 min. ESI-MS: calculated [M+H]⁺ for Ga-ProBOMB2 C₇₅H₁₁₀N₂₀O₁₉Ga 1631.7; found 1631.9. ProBOMB2 (1.36 mg, 0.869 μmol) and LuCl₃ (0.2 M, 21.7 μL, 4.3455 μmol) in 450 μL sodium acetate buffer (0.1 M, pH 4.2) was incubated at 80° C. for 30 min, and purified by HPLC using the semi-preparative column (21% acetonitrile and 0.1% TFA in water; flow rate: 4.5 mL/min. The isolated yield was 86%. Retention time: 8.6 min. ESI-MS: calculated [M+H]⁺ for Lu-ProBOMB2 C₇₅H₁₁₀N₂₀O₁₇Lu 1738.7; found 1738.7.

2.1.7 Cell Culture.

Human PC-3 prostate adenocarcinoma and murine Swiss 3T3 fibroblast cell lines were cultured and maintained in a humidified incubator (5% CO₂; 37° C.) in F-12K medium and RPMI medium (Life Technologies Corporations), respectively, and supplemented with 20% fetal bovine serum, 100 I.U./mL penicillin, and 100 μg/mL streptomycin (Life Technologies).

2.1.8 Competition Binding Assay.

The in vitro competition binding assay was modified from previously published procedures (25). PC-3 cells were seeded at 2×10⁵ cells/well in 24 well Poly-D-lysine plates 18-24 h prior to the experiment. The growth medium was replaced by 400 μL of reaction medium. Cells were incubated 30-60 min at 37° C. Non-radioactive peptides in 50 μL of decreasing concentrations (10 μM to 1 μM) and 50 μL 0.011 nM [¹²⁵l-Tyr⁴]Bombesin were added to wells. The cells were incubated with moderate agitation for 1 h at 27° C., washed thrice with ice-cold PBS, harvested by trypsinization, and measured for activity on the gamma counter. Data were analyzed using non-linear regression (one binding site model for competition assay) with GraphPad Prism 7.

2.1.9 Animal Model.

Animal experiments were approved by the Animal Ethics Committee of the University of British Columbia. Male NOD.Cg-Prkdc^{scid}∥2rg^{tm1Wjl}/SzJ (NSG) mice obtained from an in-house colony were subcutaneously inoculated with 5×10⁶ PC-3 cells (100 μL; 1:1 PBS/Matrigel), and tumors were grown for 3 weeks.

2.1.10 PET/CT Imaging.

PC-3 tumor-bearing mice were sedated (2.5% isoflurane in O₂) for i.v. injection of radiotracer (4.18±0.68 MBq) with or without 100 μg [D-Phe⁶, Leu-NHEt¹³, des-Met¹⁴]Bombesin(6-14). Mice were sedated and scanned (Siemens Inveon microPET/CT) with body temperature maintained by heating pad. The CT scan was obtained (80 kV; 500 μA; 3 bed positions; 34% overlap; 220° continuous rotation) followed by a 10 min static PET at 1 or 2 h post-injection (p.i.) of the radiotracer. PET data were acquired in list mode, reconstructed using 3-dimensional ordered-subsets expectation maximization (2 iterations) followed by a fast maximum a priori algorithm (18 iterations) with CT-based attenuation correction. Images were analyzed using the Inveon Research Workplace software (Siemens Healthineers).

2.1.11 Biodistribution.

PC-3 tumor-bearing mice were anesthetized (2.5% isoflurane in 02) for i.v. injection of radiotracer (1.47±1.17 MBq) with or without 100 μg of [D-Phe⁶, Leu-NHEt¹³, des-Met¹⁴]Bombesin(6-14). The mice were sacrificed by CO₂ inhalation at 1 h, and 2 h p.i. Blood was collected by cardiac puncture. Organs/tissues were harvested, rinsed with PBS, blotted dry, and weighed. The activity in tissues was assayed by gamma counter and expressed as the percentage injected dose per gram of tissue (% ID/g).

2.1.12 In Vivo Stability.

⁶⁸Ga-ProBOMB2 (5.9±0.3 MBq) was intravenously injected into four male NSG mice. After a 5-min and 15-min uptake period, two mice were sedated/euthanized at each timepoint, and blood was collected. The plasma was extracted from whole blood with acetonitrile, vortexed, and the supernatant separated. The plasma was analyzed with radio-HPLC (21% acetonitrile and 0.1% TFA in water; flow rate: 2.0 mL/min. Retention time of ⁶⁸Ga-ProBOMB2: 9.3 min.

2.2 Results

2.2.1 Chemistry and Radiolabeling.

The unnatural amino acid Fmoc-Leu-ψ(CH₂N)-Pro-OH was obtained with 30% yield. The radiolabeling precursor ProBOMB2 was obtained with 2.4% yields. The non-radioactive standards Ga-ProBOMB2 and Lu-ProBOMB2 were obtained in 88% and 86% yields, respectively. ⁶⁸Ga-ProBOMB2 was obtained in 48.2±0.3% decayed-corrected isolated yield and 96% radiochemical purity.

2.2.2 Binding Affinity.

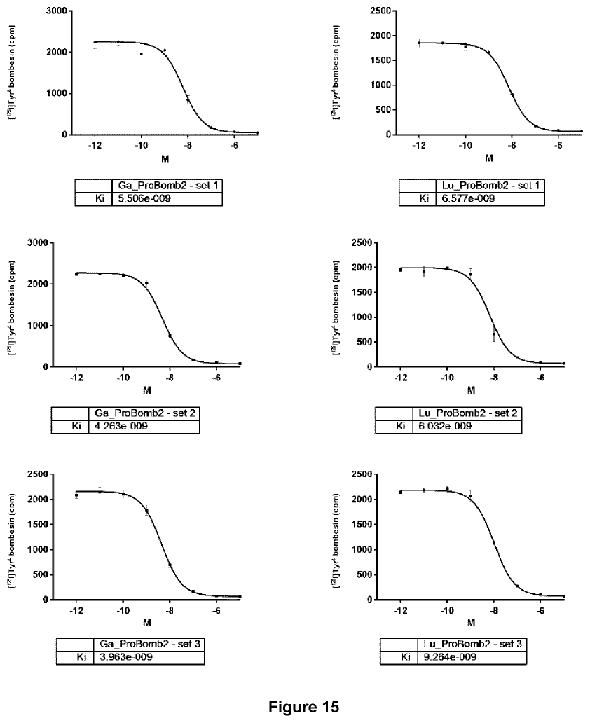

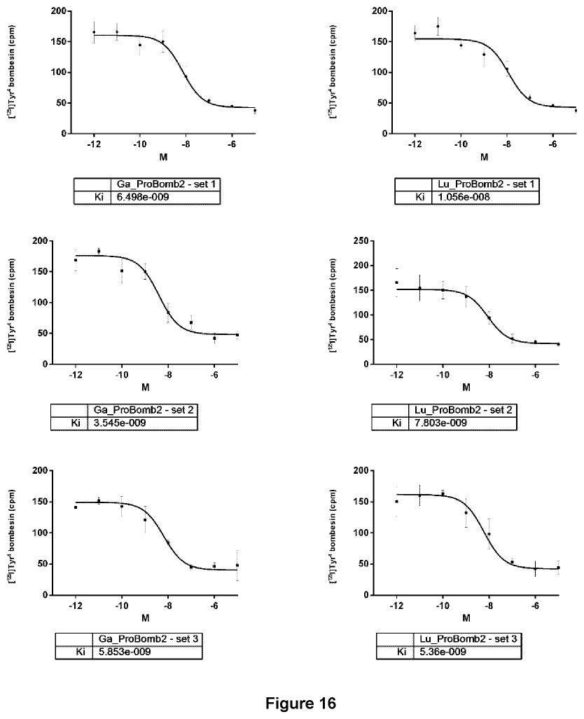

The binding affinities of Ga-ProBOMB2 and Lu-ProBOMB2 for human and murine GRPR were measured in PC-3 and Swiss 3T3 cells, respectively (FIGS. 15 and 16). The compounds successfully displaced binding of [¹²⁵I-Tyr⁴]Bombesin in a dose-dependent manner. Kᵢ values for Ga-ProBOMB2 were 4.58±0.67 and 3.97±0.76 nM for the human and murine GRPR receptor, respectively. Kᵢ values for Lu-ProBOMB2 were 7.29±1.73 and 7.91±2.60 nM for the human and murine GRPR receptor, respectively.

2.2.3 PET Imaging, Biodistribution, and Stability

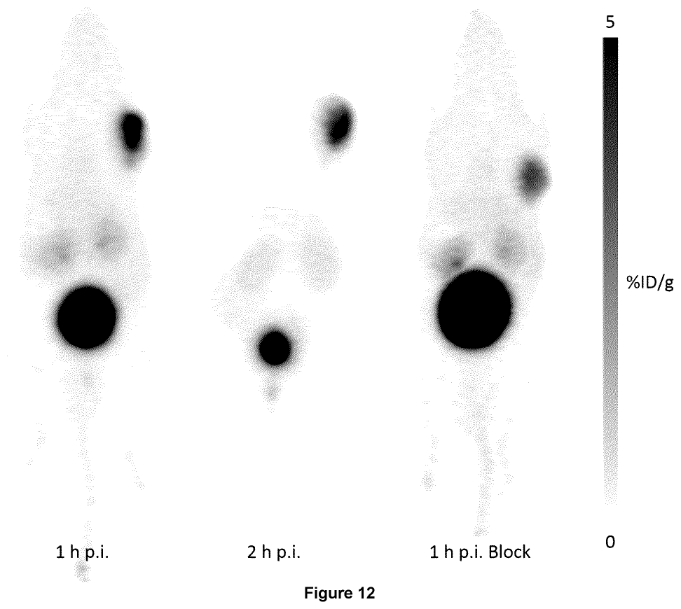

Representative maximum intensity projection PET/CT images (1 h, 1 h block, and 2 h p.i.) are shown in FIG. 12. ⁶⁸Ga-ProBOMB2 enabled clear visualization of PC-3 tumor xenografts. ⁶⁸Ga-ProBOMB2 was primarily cleared through the renal pathway. Co-injection of [D-Phe⁶, Leu-NHEt¹³, des-Met¹⁴]Bombesin(6-14) decreased average uptake of ⁶⁸Ga-ProBOMB2 in tumors by 65%.

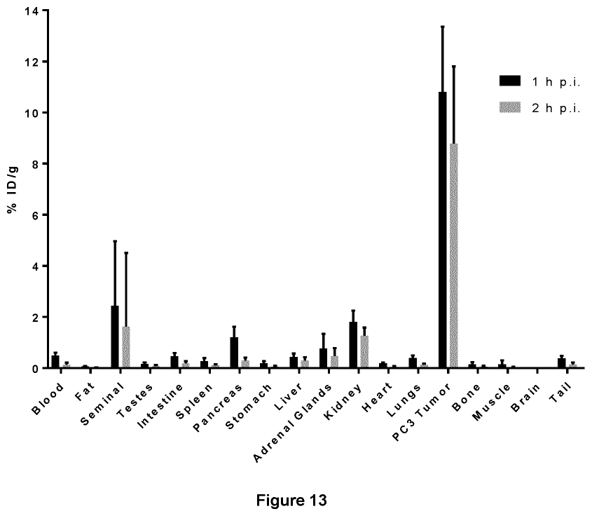

Biodistribution, uptake (% ID/g) of selected organs for ⁶⁸Ga-ProBOMB2 at 1 and 2 h p.i. were compared (FIG. 13; Table 9).

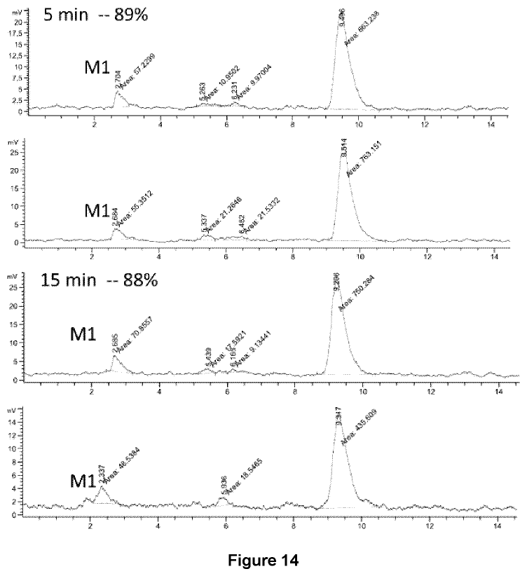

The stability of ⁶⁸Ga-ProBOMB2 was measured in plasma at 5 min and 15 min p.i. According to HPLC results (FIG. 14), ⁶⁸Ga-ProBOMB2 (t_{R}=9.35 min) was 89% intact. A minor metabolite peak was observed at t_{R}=2.72 min.

TABLE 9
Biodistribution and tumor-to-organ contrasts of ⁶⁸Ga-ProBOMB2
in PC-3 xenograft bearing mice at selected time-points.

| | 60 MIN | | | 120 MIN | | | 60 MIN BLOCKED | | |
|---|---|---|---|---|---|---|---|---|---|
| TISSUES | Mean | SD | n | Mean | SD | n | Mean | SD | n |
| BLOOD | 0.49 | 0.11 | 6 | 0.13 | 0.08 | 7 | 0.53 | 0.15 | 6 |
| FAT | 0.06 | 0.02 | 6 | 0.02 | 0.01 | 7 | 0.08 | 0.03 | 6 |
| SEMINAL | 2.44 | 2.53 | 6 | 1.63 | 2.88 | 7 | 1.95 | 2.37 | 6 |
| TESTES | 0.16 | 0.06 | 6 | 0.07 | 0.05 | 7 | 0.21 | 0.08 | 6 |
| INTESTINE | 0.46 | 0.13 | 6 | 0.19 | 0.08 | 7 | 0.34 | 0.12 | 6 |
| SPLEEN | 0.27 | 0.12 | 6 | 0.11 | 0.03 | 7 | 0.33 | 0.17 | 6 |
| PANCREAS | 1.20 | 0.42 | 6 | 0.30 | 0.11 | 7 | 0.62 | 0.19 | 6 |
| STOMACH | 0.19 | 0.08 | 6 | 0.06 | 0.03 | 7 | 0.07 | 0.02 | 6 |
| LIVER | 0.43 | 0.14 | 6 | 0.29 | 0.13 | 7 | 0.57 | 0.18 | 6 |
| ADRENAL GLANDS | 0.76 | 0.58 | 6 | 0.47 | 0.31 | 7 | 0.32 | 0.17 | 6 |
| KIDNEY | 1.81 | 0.44 | 6 | 1.27 | 0.31 | 7 | 2.22 | 0.79 | 6 |
| HEART | 0.18 | 0.04 | 6 | 0.05 | 0.02 | 7 | 0.16 | 0.03 | 6 |
| LUNGS | 0.39 | 0.10 | 6 | 0.13 | 0.04 | 7 | 0.41 | 0.09 | 6 |
| PC3 TUMOR | 10.80 | 2.56 | 6 | 8.79 | 3.01 | 7 | 3.77 | 0.92 | 6 |
| BONE | 0.15 | 0.08 | 6 | 0.06 | 0.03 | 7 | 0.13 | 0.09 | 6 |
| MUSCLE | 0.15 | 0.15 | 6 | 0.03 | 0.02 | 7 | 0.09 | 0.04 | 6 |
| BRAIN | 0.02 | 0.00 | 6 | 0.01 | 0.00 | 7 | 0.02 | 0.00 | 6 |
| RATIOS | | | | | | | | | 6 |
| TUMOR-TO-BLOOD | 22.19 | 3.51 | 6 | 75.32 | 28.59 | 7 | 7.23 | 1.40 | 6 |
| TUMOR-TO-MUSCLE | 101.99 | 41.47 | 6 | 411.17 | 220.57 | 7 | 43.96 | 8.26 | 6 |
| TUMOR-TO-KIDNEY | 6.03 | 0.88 | 6 | 6.96 | 1.89 | 7 | 1.77 | 0.39 | 6 |
| TUMOR-TO-PANCREAS | 9.32 | 1.38 | 6 | 31.05 | 9.28 | 7 | 6.17 | 1.09 | 6 |
| TUMOR-TO-LIVER | 26.18 | 6.43 | 6 | 34.99 | 16.10 | 7 | 7.33 | 2.89 | 6 |
| TUMOR-TO-BONE | 89.90 | 47.46 | 6 | 172.53 | 70.48 | 7 | 39.81 | 21.70 | 6 |

*Mice received a co-injection of 100 μg of [D-Phe⁶,Leu-NHEt¹³,des-Met¹⁴]Bombesin(6-14).

### LIST OF REFERENCES NOT CITED INLINE

1. Roesler R, Schwartsmann G. Gastrin-releasing peptide receptors in the central nervous system: Role in brain function and as a drug target. Front Endocrinol (Lausanne). 2012; 3:159.
2. Bitar K N, Zhu X X. Expression of bombesin-receptor subtypes and their differential regulation of colonic smooth muscle contraction. Gastroenterology. 1993; 105:1672-1680.
3. Weber H C. Regulation and signaling of human bombesin receptors and their biological effects. Curr Opin Endocrinol Diabetes Obes. 2009; 16:66-71.
4. Jensen R T, Battey J F, Spindel E R, Benya R V. International Union of Pharmacology. LXVIII. Mammalian Bombesin Receptors: Nomenclature, Distribution, Pharmacology, Signaling, and Functions in Normal and Disease States. Pharmacol Rev. 2008; 60:1-42.
5. Cornelio D B, Roesler R, Schwartsmann G. Gastrin-releasing peptide receptor as a molecular target in experimental anticancer therapy. Ann Oncol. 2007; 18:1457-1466.
6. Maina T, Nock B A, Kulkarni H, Singh A, Baum R P. Theranostic Prospects of Gastrin-Releasing Peptide Receptor-Radioantagonists in Oncology. PET Clin. 2017; 12:297-309.
7. Lin K S, Luu A, Baidoo K E, et al. A new high affinity technetium analogue of bombesin containing DTPA as a pharmacokinetic modifier. In: Bioconjugate Chemistry. Vol 15. American Chemical Society; 2004:1416-1423.
8. Inkster J, Lin K S, Ait-Mohand S, et al. 2-Fluoropyridine prosthetic compounds for the 18F labeling of bombesin analogues. Bloorganic Med Chem Lett. 2013; 23:3920-3926.
9. Mansi R, Minamimoto R, Macke H, lagaru A H. Bombesin-Targeted PET of Prostate Cancer. J Nucl Med. 2016; 57:67S-72S.
10. Bodei L, Ferrari M, Nunn A, et al. 177Lu-AMBA Bombesin analogue in hormone refractory prostate cancer patients: a phase I escalation study with single-cycle administrations. In: JOINT EANM-EORTC Symposium.; 2007.
11. Sah B R, Burger I A, Schibli R, et al. Dosimetry and First Clinical Evaluation of the New 18F-Radiolabeled Bombesin Analogue BAY 864367 in Patients with Prostate Cancer. J Nucl Med. 2015; 56:372-378.
12. Zang J, Mao F, Wang H, et al. 68Ga-NOTA-RM26 PET/C T in the Evaluation of Breast Cancer: A Pilot Prospective Study. Clin Nucl Med. 2018; 43:663-669.
13. Nock B A, Kaloudi A, Lymperis E, et al. Theranostic Perspectives in Prostate Cancer with the Gastrin-Releasing Peptide Receptor Antagonist NeoBOMB1: Preclinical and First Clinical Results. J Nucl Med. 2017; 58:75-80.
14. Maina T, Bergsma H, Kulkarni H R, et al. Preclinical and first clinical experience with the gastrin-releasing peptide receptor-antagonist [68Ga]SB3 and PET/CT. Eur J Nucl Med Mol Imaging. 2016; 43:964-973.
15. Kahkonen E, Jambor I, Kemppainen J, et al. In vivo imaging of prostate cancer using Ga]-labeled bombesin analog BAY86-7548. Clin Cancer Res. 2013; 19:5434-5443.
16. Wieser G, Mansi R, Grosu A L, et al. Positron emission tomography (PET) imaging of prostate cancer with a gastrin releasing peptide receptor antagonist—from mice to men. Theranostics. 2014; 4:412-419.
17. Coy D H, Heinz-Erian P, Jiang N Y, et al. Probing peptide backbone function in bombesin. A reduced peptide bond analogue with potent and specific receptor antagonist activity. J Biol Chem. 1988; 263:5056-5060.
18. Reile H, Cai R Z, Armatis P, Schally A V. New antagonists of bombesin/gastrin-releasing peptide with C-terminal Leu (CH2N)Tac-NH2. Int J Oncol. 1995; 7:749-754.
19. Bajo A M, Schally A V, Krupa M, Hebert F, Groot K, Szepeshazi K. Bombesin antagonists inhibit growth of MDA-MB-435 estrogen-independent breast cancers and decrease the expression of the ErbB-2/HER-2 oncoprotein and c-jun and c-fos oncogenes. Proc Natl Acad Sci USA. 2002; 99:3836-3841.
20. Koppán M, Halmos G, Arencibia J M, Lamharzi N, Schally A V. Bombesin/gastrin-releasing peptide antagonists RC-3095 and RC-3940-II inhibit tumor growth and decrease the levels and mRNA expression of epidermal growth factor receptors in H-69 small cell lung carcinoma. Cancer. 1998; 83:1335-1343.
21. Shirahige Y, Cai R Z, Szepeshazi K, et al. Inhibitory effect of bombesin/gastrin-releasing peptide (GRP) antagonists RC-3950-II and RC-3095 on MCF-7 MIII human breast cancer xenografts in nude mice. Biomed Pharmacother. 1994; 48:465-472.
22. Cai R Z, Qin Y, Ertl T, Schally A V. New pseudononapeptide bombesin antagonists with C-terminal Leu-psi(CH2N)Tac-NH2 show high binding affinity to bombesin/GRP receptors on CFPAC-1 human pancreatic cancer cells. Int J Oncol. 1995; 6:1165-1172.
23. Jungwirth A, Pinski J, Galvan G, et al. Inhibition of growth of androgen-independent DU-145 prostate cancer in vivo by luteinising hormone-releasing hormone antagonist cetrorelix and bombesin antagonists RC-3940-II and RC-3950-II. Eur J Cancer Part A. 1997; 33:1141-1148.
24. Lin K S, Pan J, Amouroux G, et al. In vivo radioimaging of bradykinin receptor B1, a widely overexpressed molecule in human cancer. Cancer Res. 2015; 75:387-393.
25. Pourghiasian M, Liu Z, Pan J, et al. 18F-AmBF3-MJ9: A novel radiofluorinated bombesin derivative for prostate cancer imaging. Bioorganic Med Chem. 2015; 23:1500-1506.
26. Amouroux G, Pan J, Jenni S, et al. Imaging Bradykinin 1 Receptor with 68Ga-Labeled [des-Arg10]Kallidin Derivatives: Effect of the Linker on Biodistribution and Tumor Uptake. Mol Pharm. 2015; 12:2879-2888.
27. Stabin M G, Sparks R B, Crowe E. OLINDA/EXM: the second-generation personal computer software for internal dose assessment in nuclear medicine. J Nucl Med. 2005; 46:1023-7.
28. Keenan M A, Stabin M G, Segars W P, Fernald M J. RADAR Realistic Animal Model Series for Dose Assessment. J Nucl Med. 2010; 51:471-476.
29. Morgat C, MacGrogan G, Brouste V, et al. Expression of Gastrin-Releasing Peptide Receptor in Breast Cancer and Its Association with Pathologic, Biologic, and Clinical Parameters: A Study of 1,432 Primary Tumors. J Nucl Med. 2017; 58:1401-1407.
30. Stoykow C, Erbes T, Maecke H R, et al. Gastrin-releasing Peptide Receptor Imaging in Breast Cancer Using the Receptor Antagonist (68)Ga-RM2 And PET. Theranostics. 2016; 6:1641-50.
31. Sonni I, Baratto L, lagaru A. Imaging of Prostate Cancer Using Gallium-68-Labeled Bombesin. PET Clin. 2017; 12:159-171.
32. Eder M, Schafer M, Bauder-WQst U, Haberkorn U, Eisenhut M, Kopka K. Preclinical evaluation of a bispecific low-molecular heterodimer targeting both PSMA and GRPR for improved PET imaging and therapy of prostate cancer. Prostate. 2014; 74:659-668.
33. Minamimoto R, Hancock S, Schneider B, et al. Pilot Comparison of 68Ga-RM2 PET and 68Ga-PSMA-11 PET in Patients with Biochemically Recurrent Prostate Cancer. J Nucl Med. 2016; 57:557-562.
34. Lin J T, Coy D H, Mantey S A, Jensen R T. Comparison of the peptide structural requirements for high affinity interaction with bombesin receptors. Eur J Pharmacol. 1995; 294:55-69.
35. Dalm S U, Bakker I L, de Blois E, et al. 68 Ga/177 Lu-NeoBOMB1, a Novel Radiolabeled GRPR Antagonist for Theranostic Use in Oncology. J Nucl Med. 2017; 58:293-299.
36. Fani M, Maecke H R, Okarvi S M. Radiolabeled peptides: Valuable tools for the detection and treatment of cancer. Theranostics. 2012; 2:481-501.
37. Richter S, Wuest M, Bergman C N, Krieger S, Rogers B E, Wuest F. Metabolically Stabilized 68Ga-NOTA-Bombesin for PET Imaging of Prostate Cancer and Influence of Protease Inhibitor Phosphoramidon. Mol Pharm. 2016; 13:1347-1357.
38. Nock B A, Maina T, Krenning E P, de Jong M. “To Serve and Protect”: Enzyme Inhibitors as Radiopeptide Escorts Promote Tumor Targeting. J Nucl Med. 2014; 55:121-127.
39. Gonzalez N, Nakagawa T, Mantey S A, et al. Molecular basis for the selectivity of the mammalian bombesin peptide, neuromedin B, for its receptor. J Pharmacol Exp Ther. 2009; 331(1): 265-276.
40. Dalm S U, Schrijver W A, Sieuwerts A M, et al. Prospects of Targeting the Gastrin Releasing Peptide Receptor and Somatostatin Receptor 2 for Nuclear Imaging and Therapy in Metastatic Breast Cancer. PLoS One. 2017; 12(1): e0170536.
41 Guo M, Qu X, Qin X Q. Bombesin-like peptides and their receptors: recent findings in pharmacology and physiology. Curr Opin Endocrinol Diabetes Obes. 2015; 22(1): 3-8.
42. Ischia J, Patel O, Bolton D, Shulkes A, Baldwin G S. Expression and function of gastrin-releasing peptide (GRP) in normal and cancerous urological tissues. BJU Int. 2014; 113 Suppl 2: 40-47.
43. Ramos-Alvarez et al. Peptide 2015; 72: 128-144

1. A compound of Formula Ia
R^{X}-L-Xaa¹-Gln-Trp-Ala-Val-Xaa²-His-Xaa³-ψ-Xaa⁴-NH₂ (Ia)
wherein:
R^{X} comprises a radionuclide chelator or a trifluoroborate-containing prosthetic group;
L is a linker;
Xaaⁱ is D-Phe, Cpa (4-chlorophenylalanine), D-Cpa, Tpi (2,3,4,9-tetrahydro-1H-pyrido[3,4b]indol-3-carboxylic acid), D-Tpi, Nal (naphthylalanine), or D-Nal;
Xaa² is Gly, N-methyl-Gly or D-Ala;
Xaa³ is Leu, Pro, D-Pro, or Phe;
Xaa⁴ is Pro, Phe, Tac (thiazolidine-4-carboxylic acid), Nle (norleucine), 4-oxa-L-Pro (oxazolidine-4-carboxylic acid); and
ψ represents a reduced peptide bond between Xaa³ and Xaa⁴ in which ψ is 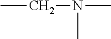
when Xaa⁴ is Pro, Tac or 4-oxa-L-Pro, or W is —CH2N(R)— when Xaa⁴ is Phe or Nle wherein R is H or C₁-C₅ linear or branched alkyl.

2. The compound of claim 1, wherein R^{X} comprises the radionuclide chelator.

3. The compound of claim 2, wherein the radionuclide chelator is selected from the group consisting of: DOTA and derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate;
hexamethylpropyleneamineoxime; and hexakis(methoxy isobutyl isonitrile).

4. The compound of claim 2, wherein the radionuclide chelator is selected from DOTA and DOTA derivatives.

5. The compound of any one of claim 2, wherein R^{X} further comprises a radiometal, a radionuclide-bound metal, or a radionuclide-bound metal-containing prosthetic group, and wherein the radiometal, the radionuclide-bound metal, or the radionuclide-bound metal-containing prosthetic group is chelated to the radionuclide-chelator complex.

6. The compound of claim 5, wherein the radiometal, the radionuclide-bound metal, or the radionuclide-bound metal-containing prosthetic group is: ⁶⁸Ga, ⁶¹Cu, ⁶⁴Cu, ⁶⁷Cu, ⁶⁷Ga ¹¹¹In, ⁴⁴Sc, ⁸⁶Y, ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, or [¹⁸F]AlF.

7. The compound of claim 5, wherein the radiometal, the radionuclide-bound metal, or the radionuclide-bound metal-containing prosthetic group is: ⁶⁸Ga, ⁶¹Cu, ⁶⁴Cu, ⁶⁷Cu, ⁶⁷Ga, ¹¹¹In, ⁴⁴Sc, ⁸⁶Y, ¹⁷⁷Lu, ⁹⁰Y, ¹⁴⁹Tb, ¹⁵²Tb, ¹⁵⁵Tb, ¹⁶¹Tb, ²²⁵Ac, ²¹³Bi, or ²¹²Bi.

8. The compound of claim 1, wherein R^{X} comprises one or more than one trifluoroborate-containing prosthetic group.

9. The compound of claim 8, wherein R^{X} comprises one or more than one R¹R²BF₃ group, wherein:
each R¹ is independently 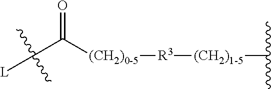
wherein each R³ is independently absent, 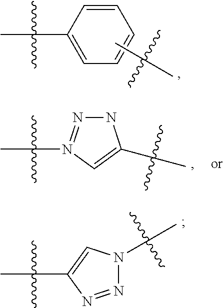
and
each R²BF₃ is independently: 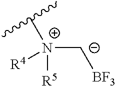
wherein each R⁴ is independently a C₁-C₅ linear or branched alkyl group and each R⁵ is independently a C₁-C₅ linear or branched alkyl group, 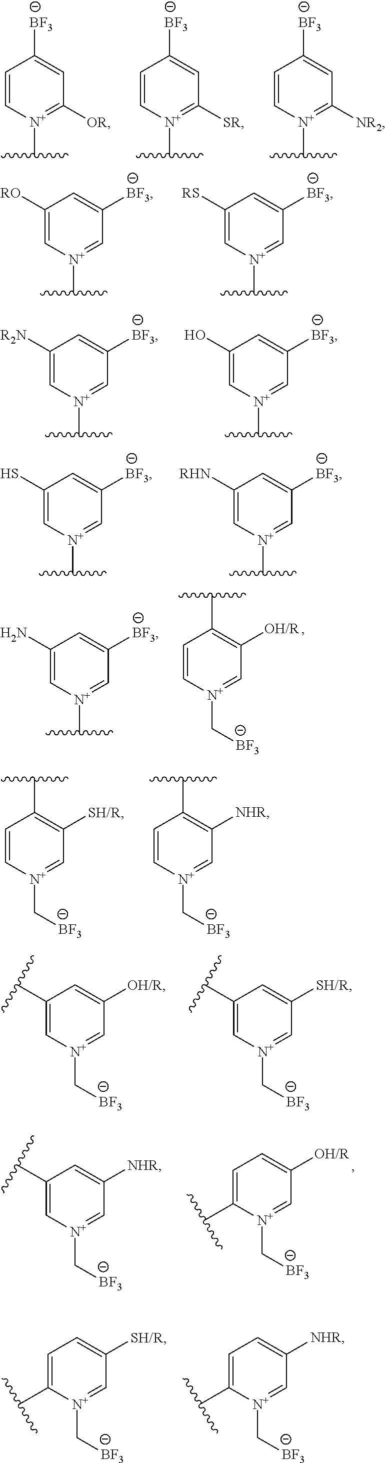 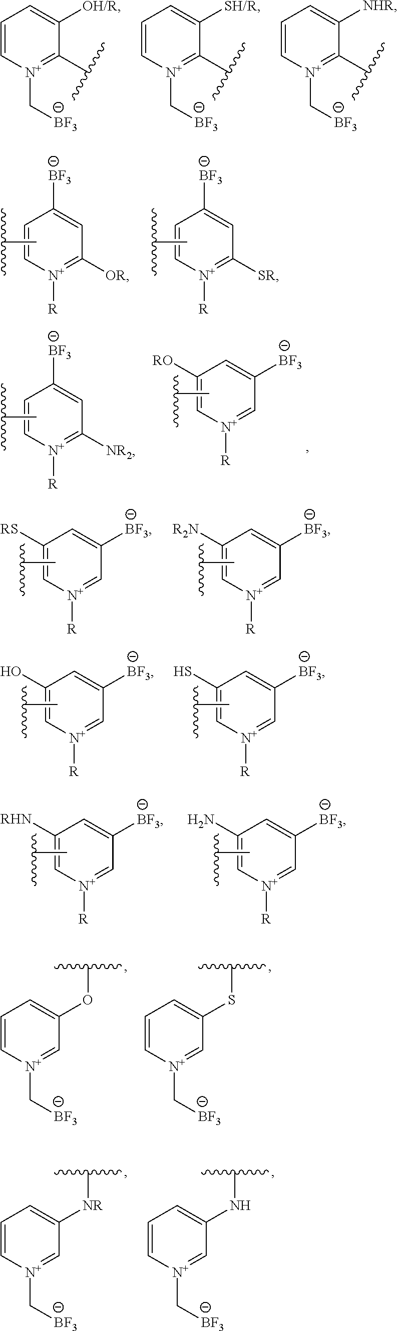 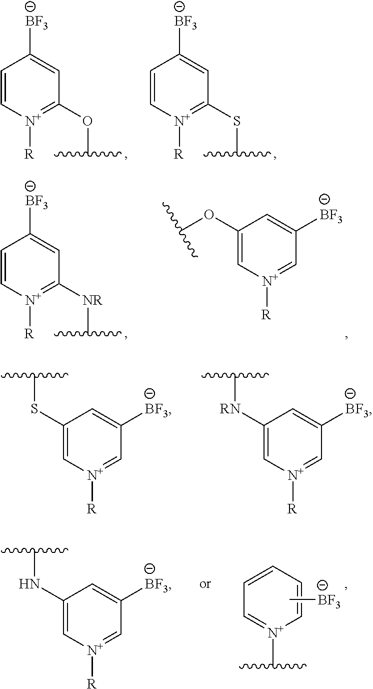
in which the R in each pyridine substituted —OR, —SR, —NR—, —NHR or —NR₂ is independently a branched or linear C₁-C₅ alkyl.

10. The compound of claim 8, wherein R^{X} comprises one or more than one R¹R²BF₃, wherein:
each R¹ is independently 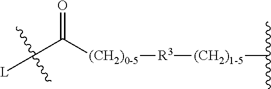
wherein each R³ is independently absent, 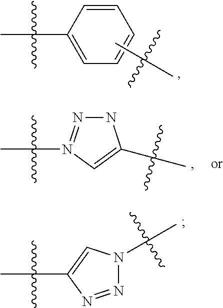
and
each R²BF₃ is independently: 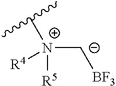
wherein each R⁴ is independently a C₁-C₅ linear or branched alkyl group and each R⁵ is independently a C₁-C₅ linear or branched alkyl group, 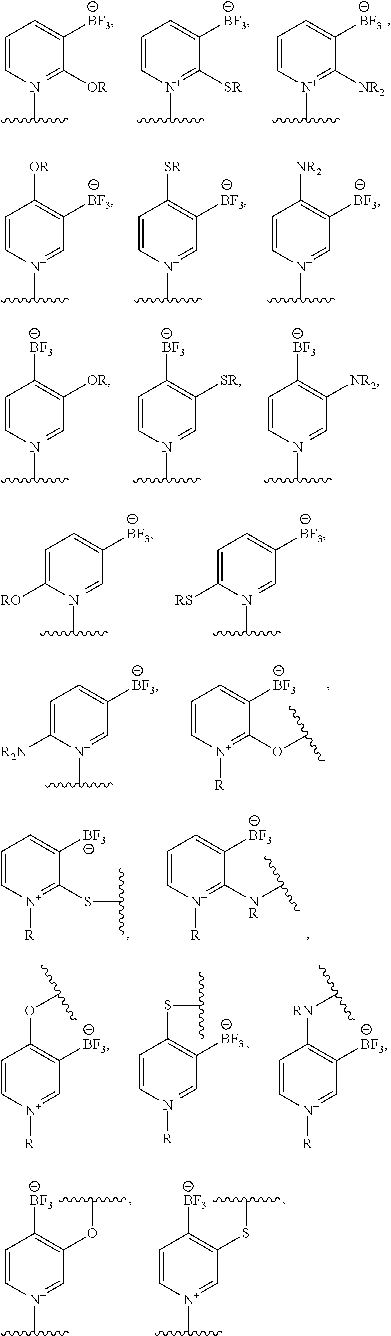 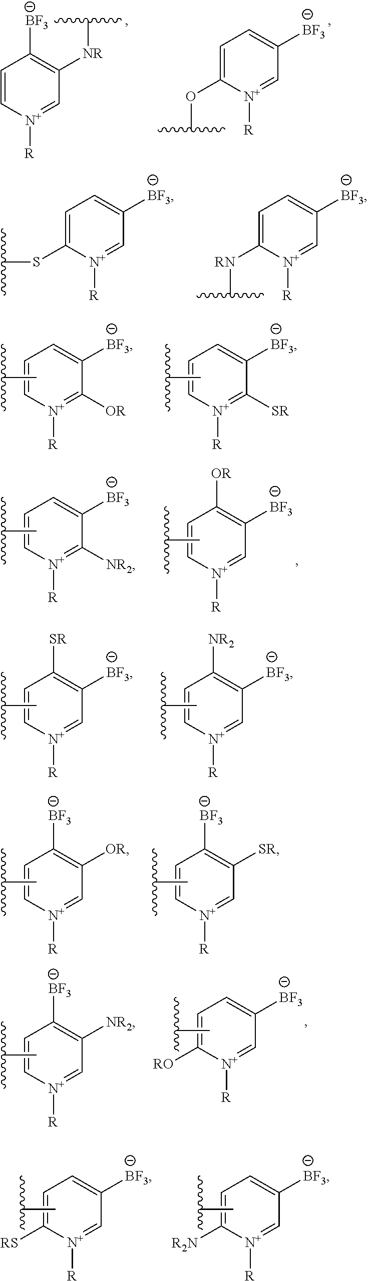 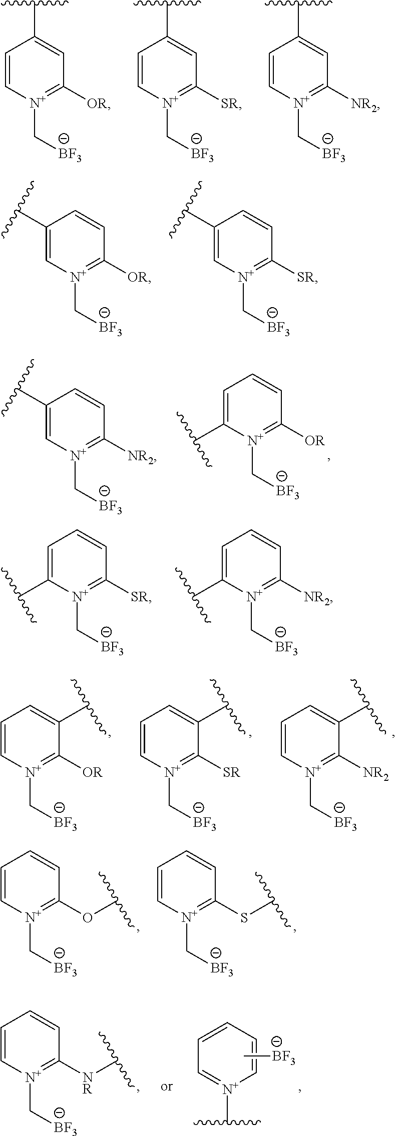
in which the R in each pyridine substituted —OR, —SR, —NR—, —NHR or —NR₂ is independently a branched or linear C₁-C₅ alkyl.

11. The compound of claim 8, wherein the R^{X} comprises a single R¹R²BF₃ group.

12. The compound of claim 8, wherein the R^{X} comprises two R¹R²BF₃ groups.

13. The compound of claim 8, wherein the trifluoroborate-containing prosthetic group comprises ¹⁸F.

14. The compound of claim 8, wherein the linker is a peptide linker (Xaa⁵)₁₋₄, wherein each Xaa⁵ is independently a proteinogenic or non-proteinogenic amino acid residue.

15. The compound of claim 1, wherein the linker is a peptide linker (Xaa⁵)₁₋₄, wherein each Xaa⁵ is independently a proteinogenic amino acid residue or is a non-proteinogenic amino acid residue, wherein each peptide backbone amino group is independently optionally methylated, and wherein each non-proteinogenic amino acid residue is independently selected from the group consisting of a D-amino acid of a proteinogenic amino acid, N^{ε}, N^{ε}, N^{ε}-trimethyl-lysine, 2,3-diaminopropionic acid (Dap), 2,4-diaminobutyric acid (Dab), ornithine (Orn), homoarginine (hArg), 2-amino-4-guanidinobutyric acid (Agb), 2-amino-3-guanidinopropionic acid (Agp), 4-(2-aminoethyl)-1-carboxymethyl-piperazine (Acp), (3-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-aminoadipic acid (2-Aad), 3-aminoadipic acid (3-Aad), cysteic acid, tranexamic acid, p-aminomethylaniline-diglycolic acid (pABzA-DIG), 4-amino-i-carboxymethyl-piperidine (Pip), NH₂(CH₂)₂O(CH₂)₂C(O)OH, NH₂(CH₂)₂[O(CH₂)₂]₂C(O)OH (dPEG2), NH₂(CH₂)₂[O(CH₂)₂]₃C(O)OH, NH₂(CH₂)₂[O(CH₂)₂]₄C(O)OH, NH₂(CH₂)₂[O(CH₂)₂]₅C(O)OH, and NH₂(CH₂)₂[O(CH₂)₂]₆C(O)OH.

16. The compound of claim 1, wherein the linker is p-aminomethylaniline-diglycolic acid (pABzA-DIG), 4-amino-(1-carboxymethyl)piperidine (Pip), 9-amino-4,7-dioxanonanoic acid (dPEG2) or 4-(2-aminoethyl)-1-carboxymethyl-piperazine (Acp).

17. (canceled)

18. The compound of claim 1, wherein: Xaa¹ is D-Phe; and/or Xaa² is Gly; and/or Xaa³ is Leu; and/or Xaa⁴ is Pro, Tac or 4-oxa-L-Pro.

19. (canceled)

20. (canceled)

21. (canceled)

22. (canceled)

23. The compound of claim 1, wherein Xaa¹ is D-Phe, Xaa² is Gly, Xaa³ is Leu, and Xaa⁴ is Pro.

24. A compound, the compound having one of the following chemical structure or a salt or solvate thereof, optionally chelated with radionuclide X:

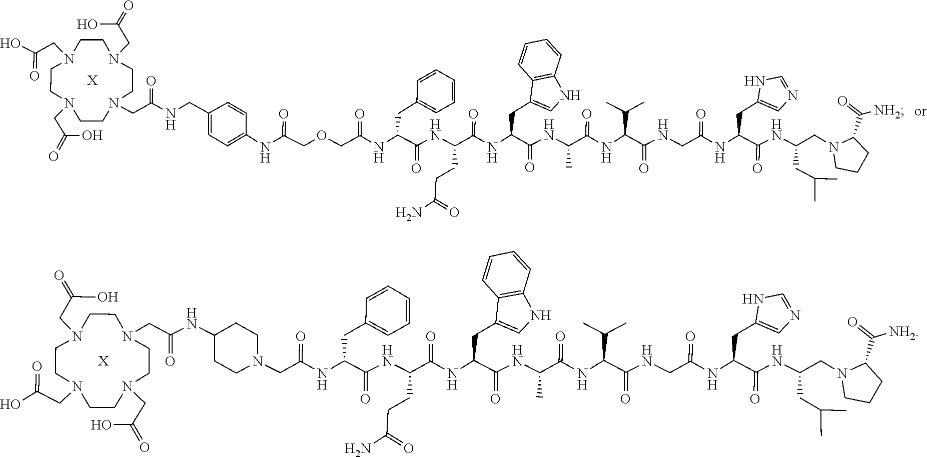

25. (canceled)

26. The compound of claim 24, where X is: ⁶⁸Ga, ⁶⁴Cu, ⁶⁷Cu, ⁶⁷Ga, ¹¹¹In, ¹⁷⁷Lu, ⁹⁰Y, or ²²⁵Ac, optionally wherein X is ⁶⁸Ga or ¹⁷⁷Lu.

27. (canceled)
